SEQUENCE LISTING (17 sequences)

SEQ ID NO: 1 - protein, 836 aa, Homo sapiens
MAGLNCGVSIALLGVLLLGAARLPRGAEAFEIALPRESNITVLIKLGTPTLLAKPCYIVISKRHITMLSIKSGERIVFTFSCQSPENHFVIEIQKNIDCMSGPCPFGEVQLQPSTSLLPTLNRTFIWDVKAHKSIGLELQFSIPRLRQIGPGESCPDGVTHSISGRIDATVVRIGTFCSNGTVSRIKMQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESNKIYVVDLSNERAMSLTIEPRPVKQSRKFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLTVILIAAVGGGVLLLSALGLIICCVKKKKKKTNKGPAVGIYNDNINTEMPRQPKKFQKGRKDNDSHVYAVIEDTMVYGHLLQDSSGSFLQPEVDTYRPFQGTMGVCPPSPPTICSRAPTAKLATEEPPPRSPPESESEPYTFSHPNNGDVSSKDTDIPLLNTQEPMEPAE

SEQ ID NO: 2 - protein, 118 aa, Artificial Sequence
  VH region sequence
EVQLQQFGAELVKPGASVKISCKASGYSFSDFNIEWLKQSHGKSLEWIGDINPNYDSTNYNQKFKGRATLTVDKSSSTAYMEVRSLTSEDTAVYYCARLGYGYAMDYWGQGTSVTVSS

SEQ ID NO: 3 - protein, 116 aa, Artificial Sequence
  VL region sequence
NIVMTQSPQSMSMSVGERVTLSCKASENVGAYVSWFQQKPDQSPKLLILAASNRYTGVPARFIGSGSATDFTLTISSVQAEDLADYHCGQSYTYPYTFGGGTKLEIKRADAAPTVS

SEQ ID NO: 4 - protein, 8 aa, Artificial Sequence
  VH CDR1
GYSFSDFN

SEQ ID NO: 5 - protein, 8 aa, Artificial Sequence
  VH CDR2
INPNYDST

SEQ ID NO: 6 - protein, 11 aa, Artificial Sequence
  VH CDR3
ARLGYGYAMDY

SEQ ID NO: 7 - protein, 6 aa, Artificial Sequence
  VL CDR1
ENVGAY

SEQ ID NO: 8 - protein, 4 aa, Artificial Sequence
  VL CDR2
  misc_feature (4)..(4): Xaa can be any naturally occurring amino acid
AASX

SEQ ID NO: 9 - protein, 9 aa, Artificial Sequence
  CL CDR3
GQSYTYPYT

SEQ ID NO: 10 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 97 aa, Homo sapiens
FPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 448 aa, Artificial Sequence
  heavy chain of chimeric 10D7 antibody
EVQLQQFGAELVKPGASVKISCKASGYSFSDFNIEWLKQSHGKSLEWIGDINPNYDSTNYNQKFKGRATLTVDKSSSTAYMEVRSLTSEDTAVYYCARLGYGYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 213 aa, Artificial Sequence
  light chain of chimeric 10D7 antibody
IVMTQSPQSMSMSVGERVTLSCKASENVGAYVSWFQQKPDQSPKLLILAASNRYTGVPARFIGSGSATDFTLTISSVQAEDLADYHCGQSYTYPYTFGGGTKLEIKRADAAPTVSIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 354 aa, Artificial Sequence
  VH nucleotide sequence antibody 10D7
GAGGTCCAGCTGCAACAGTTTGGAGCTGAGTTGGTGAAGCCTGGGGCTTCAGTGAAGATATCCTGCAAGGCTTCTGGCTACTCATTCAGTGACTTCAACATAGAATGGCTGAAGCAGAGCCATGGAAAGAGCCTTGAGTGGATTGGAGATATTAATCCTAACTATGATAGTACTAACTACAACCAGAAGTTCAAGGGAAGGGCCACATTGACTGTAGACAAGTCCTCCAGCACAGCCTACATGGAGGTCCGCAGCCTGACATCTGAGGACACTGCAGTCTATTACTGTGCAAGATTGGGGTACGGCTATGCTATGGACTACTGGGGTCAAGGAACCTCAGTCACCGTCTCCTCA

SEQ ID NO: 15 - protein, 348 aa, Artificial Sequence
  VL nucleotide sequence of antibody 10D7
AACATTGTGATGACCCAATCTCCCCAATCCATGTCCATGTCAGTAGGAGAGAGGGTCACCTTGAGCTGCAAGGCCAGTGAGAATGTGGGTGCTTATGTATCCTGGTTTCAACAGAAACCAGACCAGTCTCCTAAATTGCTGATATTAGCGGCATCCAACCGGTACACTGGGGTCCCCGCTCGTTTCATAGGCAGTGGATCTGCAACAGACTTCACTCTGACCATCAGCAGTGTGCAGGCTGAAGACCTTGCAGATTATCACTGTGGACAGAGTTATACCTATCCGTACACGTTCGGAGGGGGGACCAAACTGGAAATAAAACGGGCTGATGCTGCACCAACTGTATCC

SEQ ID NO: 16 - DNA, 48 bp, Artificial Sequence
  control shRNA
cctaaggttaagtcgccctcgctcgagcgagggcgacttaaccttagg

SEQ ID NO: 17 - DNA, 59 bp, Artificial Sequence
  CDCP1 shRNA
ccgggctcataagagcatcggtttactcgagtaaaccgatgctcttatgagcttttttg

(19) United States
(12) Patent Application Publication
(10) Pub. No.: US 2022/0389113 A1
(43) Pub. Date: Dec. 8, 2022

(54) BINDING PROTEINS TO CUB DOMAIN-CONTAINING PROTEIN (CDCP1)

(71) Applicants: COMMONWEALTH SCIENTIFIC AND INDUSTRIAL RESEARCH ORGANISATION, Acton, Australian Capital Territory (AU); THE SCRIPPS RESEARCH INSTITUTE, La Jolla, CA (US); THE UNIVERSITY OF QUEENSLAND, St. Lucia, Queensland (AU)

(72) Inventors: Simon PUTTICK, Pullenvale, Queensland (AU); Thomas KRYZA, Woolloongabba, Queensland (AU); John HOOPER, Woolloongabba, Queensland (AU); Yaowu HE, Woolloongabba, Queensland (AU); Brittney HARRINGTON, Woolloongabba, Queensland (AU); James QUIGLEY, La Jolla, CA (US); Elena DERYUGINA, La Jolla, CA (US)

(21) Appl. No.: 17/774,796

(22) PCT Filed: Nov. 6, 2020
(86) PCT No.: PCT/AU2020/051216

§ 371 Date: May 5, 2022

(30) Foreign Application Priority Data
Nov. 6, 2019 (AU) 2019904177

(51) Int. Cl. C07K 16/28; G01N 33/574; A61K 47/68; A61K 51/10; A61P 35/00; A61P 1/18; C07K 16/30

(52) U.S. Cl. CPC C07K 16/2896; G01N 33/57492; A61K 47/6803; A61K 47/6849; A61K 51/1027; A61P 35/00; A61P 1/18; C07K 16/30; C07K 2317/77; A61K 2039/505

(57) ABSTRACT

The present disclosure relates to anti-CDCP1 antibodies, and antigen binding fragments thereof that specifically bind to the full length and cleaved forms CUB domain-containing protein 1 (CDCP1), and conjugates comprising anti-CDCP1 antibodies and uses thereof for treatment and detection of cancer.

### INCORPORATION BY REFERENCE

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

This application claims priority to AU2019904177 filed 6 Nov. 2019, the entire contents of which are incorporated by reference.

The entire content of the electronic submission of the sequence listing is incorporated by reference in its entirety for all purposes.

### FIELD OF THE DISCLOSURE

The present disclosure relates to anti-CDCP1 antibodies, and antigen binding fragments thereof that specifically bind to the full length and cleaved forms CUB domain-containing protein 1 (CDCP1), and conjugates comprising anti-CDCP1 antibodies and uses thereof for treatment and detection of cancer.

### BACKGROUND OF THE DISCLOSURE

Human CDCP1 (CUB domain-containing protein 1, CD318, SIMA135, TRASK, GP140) as well as variants with mutation R525Q and/or mutation G709D is a single transmembrane receptor protein containing three extracellular regions that have homology to CUB domains. The 135 kDa receptor is heavily glycosylated and can be proteolytically cleaved to 70 kDa in cell lines and tissues (Hooper J D et al., (2003) Oncogene 22(12):1783-1794; He Y et al (2010) J Biol Chem 285(34):26162-73). The cytoplasmic domain of CDCP1 includes five conserved tyrosine residues that act as a substrate of Src Family Kinases (SFK) such as Src, Fyn, and Yes for subsequent phosphorylation. Elevated expression of CDCP1 correlates with poor outcome in renal, lung, colorectal, pancreatic, breast and clear cell ovarian cancer. Data from preclinical models suggests that it may have use as a therapeutic target for cancer treatment (Fukuchi K et al., (2010) Mol Pharm 7(1):245-253; He Y et al., (2016) Oncogene 35(4):468-78). Based on data from in vitro and animal models, CDCP1 is functionally important for each of these cancers and others by promoting cell survival, dissemination, and resistance to chemotherapy and targeted agents.

The receptor relays cancer promoting signals via other receptors, such as EGFR, HER2 and β1 integrin, as well as key intracellular signal transducers including Src, PKC6, Akt and FAK. After removal of its 29 residue signal peptide, CDCP1 spans 807 residues including a 637 residue amino-terminal extracellular domain (ECD), a 20 residue transmembrane domain, and a 150 residue carboxyl-terminal intracellular domain (Uekita T, et al., (2011) Cancer Sci. 102(11):1943-8). The kinase Src is a key regulator of CDCP1-mediated signalling in pathological settings including cancer. CDCP1 is phosphorylated by Src at tyrosine 734 (Y734) and then Y743 and Y762 (Benes C H, et al., (2005) Cell. 121(2):271-8027), which occurs in response to reduced cell adhesion during mitosis, physiological cell shedding, cell de-adhesion, cleavage of 135 kDa CDCP1 to generate a 70 kDa cell retained fragment, and oncogenic transformation. Src phosphorylation of CDCP1 results in docking of PKC6 and signal transduction via the kinase FAK, matrix binding integrins, the receptor tyrosine kinase HER2 and Akt pro-survival signalling.

### CDCP1 and Ovarian Cancer

CDCP1 is a potential target in epithelial ovarian cancer (EOC) for therapeutic mAbs as it is expressed on the cell surface of the malignant component of the vast majority of these tumours and is not expressed by normal ovary and fallopian tube (He Y et al., (2016) Oncogene 35(4):468-78; Harrington B S et al., Br J Cancer (2016) 114(4):417-26). Also, it is functionally important, promoting EOC cell migration, survival, spheroid formation and chemotherapy resistance in vitro, and tumour growth and metastasis in vivo (Dong Y, et al., (2012) J Biol Chem. 287(13):9792-803; Adams M N et al Oncogene (2015) 35(4):468-78; He Y et al., (2016) Oncogene 35(4):468-78; Harrington B S et al., Br J Cancer (2016) 114(4):417-26).

### CDCP1 and Pancreatic Cancer

Pancreatic-ductal adenocarcinoma (PDAC), the most common form of pancreatic cancer, is predicted by 2030 to become the second leading cause of cancer-related death in developed countries. Lack of clinical symptoms leading to diagnosis at late stage, difficulties in accurately staging disease burden, and limited treatment options contribute to the high mortality rate. Surgery significantly extends survival, while marginal improvements are achieved with chemo- and radio-therapy. Five year survival is abysmal at less than 10%.

Limited experimental evidence suggests that CDCP1 is also important in PDAC. Immunohistochemical analysis of a single patient cohort that was not sub-classified into histological subtypes, revealed CDCP1 expression in all 145 analysed pancreatic cancer cases with no expression in normal pancreas (Miyazawa Y, et al., (2010) Cancer Res. 70(12):5136-46). Low expression was apparent in 92 of these cases (63%) and 53 (37%) displayed high CDCP1 expression. Elevated expression correlated with shorter overall survival although the result only achieved marginal significance possibly due to a lack of histological sub-classification of the patient cohort (Miyazawa et al supra). Transient silencing of CDCP1 demonstrated that it regulates pancreatic cancer cell line migration, invasion, and extracellular matrix degradation (Miyazawa et al supra). Upregulation of CDCP1 expression in response to transformation by RAS, an oncogene somatically mutated in more than 90% of PDAC tumours (Wood L D, Hruban R H., (2012) Cancer J 18: 492-501), is also suggestive of a functional role for CDCP1 in this cancer.

Antibody drug conjugates (ADC) are a developing class of therapeutic comprising an antibody conjugated to a cytotoxic drug via a chemical linker. Such conjugates combine the binding capabilities of an antibody with a drug, whereby the antibody is used to deliver the drug to a tumour cell by virtue of its binding to a cell surface receptor present on the tumour cell and subsequent internalisation of the ADC-receptor complex. There is a need in the art for therapeutic agents with the ability to target CDCP1 and facilitate killing of CDCP1-expressing cancer cells, such as EOC and PDAC.

### SUMMARY OF THE DISCLOSURE

To define the potential clinical utility of CDCP1 in cancer, the present inventors examined its expression in ovarian and pancreatic cancer cells, and directly disrupted its role in cell lines and patient-derived cells in vitro and in mouse models using a function blocking monoclonal antibody 10D7 described herein. Assays revealed a novel proteolytic mechanism that impacts CDCP1 in cancer cells. Critically, this proteolytic cleavage does not alter the ability of the antibody 10D7 to be an effective agent for anti-CDCP1 antibody-mediated delivery of imaging radionuclides and a cytotoxin to cancer cells in vitro and in vivo. The inventors show that CDCP1 is functionally important in certain cancers, as exemplified herein in ovarian and pancreatic ductal adenocarcinoma (PDAC) and that it has clinical potential as a prognostic marker and as a target for delivery of agents for detection and treatment of this malignancy.

The antibodies of the present disclosure are unique in their ability to be rapidly internalised following binding to the CDCP1 receptor. Moreover, the antibodies are able to bind to both the native uncleaved receptor as well as receptor following proteolytic processing (cleaved receptor). This provides certain advantages over prior art antibodies, for example increased sensitivity which potentially translates to lower treatment dosages and more sensitive detection for imaging.

The present disclosure is based on the generation of antibody-based agents, specifically (anti-CDCP1 antibodies, including labelled anti-CDCP1 antibodies and antibody-drug conjugates (ADCs)) useful in the detection, prognosis and/or treatment of CDCP1-expressing cancers, for example ovarian, pancreatic and colorectal cancers.

In a first aspect, the disclosure provides a CDCP1 binding protein comprising an antigen-binding domain which binds specifically to human CDCP1. In one example, the antigen-binding domain is an antigen-binding domain of an anti-CDCP1 antibody or antigen-binding fragment thereof. In one example, the CDCP1 binding protein is an anti-CDCP1 recombinant or synthetic antibody. In another example the CDCP1 binding protein is a monoclonal antibody or antigen-binding fragment thereof. In another example, the CDCP1 binding protein is a chimeric, human, humanised, primatised or deimmunised antibody. In one example, the antibody or antigen-binding fragment thereof is isolated.

In one example, the anti-CDCP1 antibody, or antigen-binding fragment thereof binds to a region of human CDCP1 spanning residues 30 to 358 of the human CDCP1 sequence described in UniProt reference Q9H5V8 or spanning residues 30 to 341 of the human CDCP1 isoform (NCBI Reference sequence NP_835488). In a particular example, the anti-CDCP1 antibody or antigen-binding fragment thereof binds to the sequence set forth in SEQ ID NO:1 or part thereof, more particularly, residues 30 to 358 of SEQ ID NO:1.

In one example, the anti-CDCP1 antibody or antigen-binding fragment thereof binds to the same epitope on human CDCP1 or to an epitope on human CDCP1 that overlaps with the epitope bound by antibody 10D7 comprising a variable heavy (VH) chain having the sequence set forth in SEQ ID NO:2 and a variable light (VL) chain comprising the sequence set forth in SEQ ID NO:3. In one example, the competing antibody is not anti-CDCP1 antibody 41-2 described in Deryugina El., et al (2009) Mol Cancer Res 7(8):1197-211. In another example, the competing antibody is not 25A11 described in in Siva A C et al., (2008) Cancer Res 68:3759-66. In another example, the competing antibody is not CD318.

In one example, the anti-CDCP1 antibody or antigen-binding fragment thereof binds to human CDCP1 in glycosylated or non-glycosylated form.

In one example, the anti-CDCP1 antibody or antigen-binding fragment thereof binds to SEQ ID NO:1, or to a region within amino acids 30 to 358 of SEQ ID NO:1 at a similar or substantially the same level, or with a similar or substantially the same affinity as the antibody designated 10D7.

In one example, the antibody according to any aspect described herein is not anti-CDCP1 antibody 41-2 described in Deryugina El., et al (2009) Mol Cancer Res 7(8):1197-211. In another example, the antibody is not 25A11 described in in Siva A C et al., (2008) Cancer Res 68:3759-66. In another example, the antibody is not CD318.

In one example, the anti-CDCP1 antibody or antigen-binding fragment thereof binds to CDCP1 with an affinity dissociation constant (K_{D}) of about 1000 nM or less, such as about 500 nM or less, about 200 nM or less, about 100 nM or less, about 80 nM or less, about 60 nM or less, about 50 nM or less, about 40 nM or less, about 20 nM or less, about 10 nM or less, about 5 nM or less, about 2 nM or less, about 1.5 nM or less, or about 1 nM or less. In one example, the KD is between about 0.01 nM to about 2 nM, such as between about 0.05 nM to about 1 nM, for example, between about 0.1 nM to about 1 nM, for example, between about 0.3 nM to about 1 nM. In another example, the KD is about 0.4 to 0.5 nM. In a particular example, the K_{D} is about 0.44 nM.

The antibodies of the present disclosure when bound to CDCP1 were observed to be rapidly internalised in cancer cells expressing the CDCP1 receptor in either its cleaved or uncleaved form.

In one example, the anti-CDCP1 antibody or antigen-binding fragment thereof binds to the uncleaved CDCP1 receptor. In another example, the anti-CDCP1 antibody or antigen-binding receptor fragment thereof binds to the cleaved CDCP1 receptor wherein the amino terminal cleaved portion remains tethered to the cell surface via the carboxyl terminal fragment of the receptor.

In one example, the anti-CDCP1 antibody or antigen-binding fragment thereof, including labelled antibody and antibody-drug conjugates comprising the anti-CDCP1 antibody are capable of being internalised when bound to the cleaved or uncleaved receptor. In one example, both the antibody and receptor are internalised when bound. In another example, the labelled antibody and/or antibody-drug conjugates comprising the anti-CDCP1 antibody are capable of inducing cell death of cells endogenously expressing cleaved or uncleaved CDCP1.

In one example, the anti-CDCP1 antibody or antigen-binding fragment thereof substantially improves the efficacy of chemotherapy compared to administration of the chemotherapy or antibody alone. In another example, the combination of anti-CDCP1 and chemotherapy reduced tumour burden by at least 25%, at least 30%, at least 40%, at least 50%, or at least 60% compared to anti-CDCP1 antibody alone. In one example, the tumour is pancreatic cancer cells. In another example, the chemotherapy is gemcitabine.

In one example, the anti-CDCP1 antibody or antigen-binding fragment thereof substantially reduces survival of cancer cells when combined with chemotherapy compared to the chemotherapy or antibody alone. In another example, the anti-CDCP1 antibody or antigen-binding fragment thereof combined with chemotherapy reduces survival of cancer cells by at least 35%, at least 40%, at least 45%, at least 50%, or at least 55% compared to chemotherapy and control IgG.

In one example, the half-maximal inhibitory concentration (IC₅₀) of cancer cells when treated with the anti-CDCP1 antibody is reduced when combined with chemotherapy by at least 35%, at least 40%, at least 45%, at least 50%, or at least 55% compared to cancer cells treated with chemotherapy and control IgG.

In one example, the cancer cells are pancreatic cancer cells. In another example, the cancer cells are ovarian cancer cells. In another example the cancer cells are colorectal cancer cells. In another example, the chemotherapy is gemcitabine.

The antibodies of the disclosure according to any aspect comprise one or more of the following functional features selected from:

(i) ability to induce rapid internalisation of the antibody/CDCP1 complex when bound to CDCP1;

(ii) rapid binding to the CDCP1 receptor; and

(iii) binding to the CDCP1 receptor in either its cleaved or uncleaved form.

In a second aspect, the disclosure provides an isolated anti-CDCP1 antibody or antigen-binding fragment thereof comprising a heavy chain variable (VH) region having complementarity determining region (CDR) sequences comprising the sequence set forth in GYSFSDFN (SEQ ID NO:4), INPNYDST (SEQ ID NO:5), ARLGYGYAMDY (SEQ ID NO:6) respectively and/or a light chain variable (VL) region having complementarity determining region (CDR) sequences comprising the sequence set forth in ENVGAY (SEQ ID NO:7), AAS (SEQ ID NO:8) and GQSYTYPYT (SEQ ID NO:9).

In one example, the anti-CDCP1 antibody or antigen-binding fragment thereof comprises a VH comprising a sequence which is at least 86% identical to the sequence set forth in SEQ ID NO:2 and/or a VL comprising a sequence which is at least 92% identical to the sequence set forth in SEQ ID NO:3.

The sequence of SEQ ID NO:2 (heavy chain variable region) comprises the sequence set forth as:

EVQLQQFGAELVKPGASVKISCKASGYSFSDFNIEWLKQSHGKSLEWIG
DINPNYDSTNYNQKFKGRATLTVDKSSSTAYMEVRSLTSEDTAVYYCAR
LGYGYAMDYWGQGTSVTVSS.

The sequence of SEQ ID NO:3 (light chain variable region) comprises the sequence set forth as:

NIVMTQSPQSMSMSVGERVTLSCKASENVGAYVSWFQQKPDQSPKLLIL
AASNRYTGVPARFIGSGSATDFTLTISSVQAEDLADYHCGQSYTYPYTF
GGGTKLEIKRADAAPTVS.

In one example, the VH comprises a sequence which is at least 88%, 90%, 92%, 95%, 97%, 98%, 99% or 99.5% identical to the sequence set forth in SEQ ID NO:2.

In one example, the VL comprises a sequence which is at least 94%, 95%, 97%, 98%, 99% or 99.5% identical to the sequence set forth in SEQ ID NO:3.

In a third aspect, the disclosure provides an anti-CDCP1 antibody or antigen-binding protein thereof, comprising a heavy chain variable region (VH) comprising the sequence set forth in SEQ ID NO:2 and a light chain variable region (VL) comprising the sequence set forth in SEQ ID NO:3. In one example, the VH when paired with the VL specifically binds to human CDCP1 and/or Cynomolgus CDCP1.

In one example, the anti-CDCP1 antibody further comprises a heavy chain constant region and/or a light chain constant region sequence. In a further example, the heavy and light chain constant regions are mouse sequences. In another example, the heavy and light chain constant region sequences are human sequences. In another example, the anti-CDCP1 antibody is a chimeric antibody comprising mouse heavy and light chain variable region sequences and human heavy and light constant region sequences. In one example, the CDCP1 binding protein is a humanised or fully human antibody. In one example, the heavy chain constant region sequence comprises the sequence set forth in SEQ ID NO:10. In one example, the light chain constant region comprises the sequence set forth in SEQ ID NO:11.

In one example, the variable region framework and CDR sequences of the anti-CDCP1 antibody or antigen-binding fragment thereof are defined by the IMGT numbering system.

The antibodies of the present disclosure may belong to any class, including IgM, IgG, IgE, IgA, IgD, or subclass. Exemplary subclasses for IgG are IgG1, IgG2, IgG3 and IgG4.

In one example, the anti-CDCP1 antibody or antigen-binding fragment thereof is recombinant. In one example the anti-CDCP1 antibody or antigen-binding fragment thereof is synthetic.

In a fourth aspect, the disclosure provides an anti-CDCP1 antibody comprising heavy chain variable and constant region sequences at least 95% identical to the sequence set forth in SEQ ID NO:12, or a sequence having at least 96%, 97%, 98% or 99% identity to SEQ ID NO:12, and/or light chain variable and constant region sequences at least 95% identical to the sequence set forth in SEQ ID NO:13, or a sequence having at least 96%, 97%, 98% or 99% identity to SEQ ID NO:13.

In one example, the anti-CDCP1 antibody comprises heavy chain variable and constant region sequences set forth in SEQ ID NO:12 and/or light chain variable and constant region sequences set forth in SEQ ID NO:13.

The sequence of SEQ ID NO:12 comprises the sequence set forth as:

EVQLQQFGAELVKPGASVKISCKASGYSFSDFNIEWLKQSHGKSLEWIG
DINPNYDSTNYNQKFKGRATLTVDKSSSTAYMEVRSLTSEDTAVYYCAR
LGYGYAMDYWGQGTSVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

The sequence of SEQ ID NO:13 comprises the sequence set forth as:

IVMTQSPQSMSMSVGERVTLSCKASENVGAYVSWFQQKPDQSPKLLILA
ASNRYTGVPARFIGSGSATDFTLTISSVQAEDLADYHCGQSYTYPYTFG
GGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC.

In another example of the disclosure, the VL and VH are in separate polypeptide chains. For example, the anti-CDCP1 antibody or antigen-binding fragment thereof is:

(i) a diabody;

(ii) a triabody;

(iii) a tetrabody;

(iv) a Fab;

(v) a F(ab′)2;

(vi) a Fv; or

(vii) at least one of (i) to (vi) linked to a heavy chain constant region or an Fc or a heavy chain constant domain (CH) 2 and/or CH3.

In a fifth aspect, the anti-CDCP1 antigen-binding fragment is selected from:

(i) a single chain Fv fragment (scFv); or

(ii) a dimeric scFv (di-scFv).

In one example, the anti-CDCP1 antibody according to the disclosure is glycosylated (if it comprises an Fc) with a sugar chain at Asn297 whereby the amount of fucose within said sugar chain is 65% or lower (Numbering according to Kabat). In another embodiment is the amount of fucose within said sugar chain is between 5% and 65%, preferably between 20% and 40%. “Asn297” according to the invention means amino acid asparagine located at about position 297 in the Fc region. Based on minor sequence variations of antibodies, Asn297 can also be located some amino acids (usually not more than ±3 amino acids) upstream or downstream of position 297, i.e. between position 294 and 300. In one example, the glycosylated antibody according to the invention the IgG subclass is of human IgG1 subclass, of human IgG1 subclass with the mutations L234A and L235A or of IgG3 subclass. In a further example the amount of N-glycolylneuraminic acid (NGNA) is 1% or less and/or the amount of N-terminal alpha-1,3-galactose is 1% or less within said sugar chain. The sugar chains show preferably the characteristics of N-linked glycans attached to Asn297 of an antibody recombinantly expressed in a CHO cell.

The present disclosure also contemplates antibodies with one or more amino acid modifications in the Fc region. These modification may include those intended to improve antibody stability or half-life. Examples include the known “YTE” modification.

In a sixth aspect, the disclosure provides an antibody, or antigen-binding fragment thereof that binds to the same epitope as the anti-CDCP1 antibody described herein. In one example, the antibody according to this aspect is not anti-CDCP1 antibody 41-2 described in Deryugina El., et al (2009) Mol Cancer Res 7(8):1197-211. In another example, the antibody is not 25A11 described in in Siva A C et al., (2008) Cancer Res 68:3759-66. In another example, the antibody is not CD318.

In an seventh aspect, the disclosure provides an isolated nucleic acid encoding the anti-CDCP1 antibody or antigen-binding fragment thereof described herein. In one example, the nucleic acid sequence is a VH sequence comprising the sequence set forth in SEQ ID NO:14. In one example, the nucleic acid sequence is a VL sequence, comprising the sequence set forth in SEQ ID NO:15.

In an eighth aspect, the disclosure provides an expression construct comprising the nucleic acid encoding the antibody described herein operably linked to a promoter. Such an expression construct can be in a vector, e.g., a plasmid.

In some examples, the expression construct may comprise a promoter linked to a nucleic acid encoding that polypeptide chain.

In examples directed to multiple polypeptides that form the anti-CDCP1 antibody, an expression construct of the disclosure comprises a nucleic acid encoding one of the polypeptides (e.g., comprising a VH) operably linked to a promoter and a nucleic acid encoding another of the polypeptides (e.g., comprising a VL) operably linked to another promoter.

In another example, the expression construct is a bicistronic expression construct, e.g., comprising the following operably linked components in 5′ to 3′ order:

(i) a promoter
(ii) a nucleic acid encoding a first polypeptide;
(iii) an internal ribosome entry site; and
(iv) a nucleic acid encoding a second polypeptide.

For example, the first polypeptide comprises a VH and the second polypeptide comprises a VL, or the first polypeptide comprises a VL and the second polypeptide comprises a VH.

The present disclosure also contemplates separate expression constructs one of which encodes a first polypeptide (e.g., comprising a VH and optionally heavy chain constant region or part thereof) and another of which encodes a second polypeptide (e.g., comprising a VL and optionally light chain constant region). For example, the present disclosure also provides a composition comprising:

(i) a first expression construct comprising a nucleic acid encoding a polypeptide (e.g., comprising a VH operably linked to a promoter); and
(ii) a second expression construct comprising a nucleic acid encoding a polypeptide (e.g., comprising a VL operably linked to a promoter),

wherein the first and second polypeptides associate to form an anti-CDCP1 antibody of the present disclosure.

In a ninth aspect, the disclosure provides an isolated cell expressing the anti-CDCP1 antibody of the present disclosure or a recombinant cell genetically-modified to express the anti-CDCP1 antibody of the disclosure. In one example, the cell is an isolated hybridoma. In another example, the cell comprises the nucleic acid of, or the expression construct of, the disclosure or:

(i) a first expression construct comprising a nucleic acid encoding a polypeptide (e.g., comprising a VH) operably linked to a promoter; and
(ii) a second expression construct comprising a nucleic acid encoding a polypeptide (e.g., comprising a VL) operably linked to a promoter,

wherein the first and second polypeptides associate to form an anti-CDCP1 antibody of the present disclosure.

Expression is performed in appropriate prokaryotic or eukaryotic host cells like CHO cells, NS0 cells, SP2/0 cells, HEK293 cells, COS cells, yeast, or E. coli cells, and the antibody is recovered from the cells (supernatant or cells after lysis).

Recombinant production of antibodies is well-known in the art and described, for example, in the review articles of Makrides, S. C., Protein Expr. Purif. 17 (1999) 183-202; Geisse, S. et al., Protein Expr. Purif. 8 (1996) 271-282; Kaufman, R. J., Mol. Biotechnol. 16 (2000) 151-160; Werner, R. G., Drug Res. 48 (1998) 870-880).

In an tenth aspect, the disclosure provides an anti-CDCP1 antibody or antigen-binding fragment thereof as described herein, conjugated to a detectable label. In some examples the detectable label is a fluorescent agent or radioisotope. In one example, the detectable label is one used in PET imaging.

In a eleventh aspect, the disclosure provides an anti-CDCP1 antibody or antigen-binding fragment thereof as described herein coupled to a moiety. In one example, the moiety is selected from the group consisting of an anti-apoptotic agent, a mitotic inhibitor, an anti-tumour antibiotic, an immunomodulating agent, a nucleic acid for gene therapy, an anti-angiogenic agent, an anti-metabolite, a toxin, a boron-containing agent, a chemoprotective agent, a hormone agent, an anti-hormone agent, a corticosteroid, a photoactive therapeutic agent, an oligonucleotide, a radiosensitizer, a topoisomerase inhibitor, a tyrosine kinase inhibitor, or polyethylene glycol (PEG). In some examples, the coupling is via a linker.

In one example, the moiety is conjugated to the antibody, or antigen-binding fragment thereof. In a further example the conjugation is via a linker. In another example, the linker is a cleavable linker. In yet other example, the linker is a non-cleavable linker.

In a twelfth aspect, the disclosure provides an antibody drug conjugate (ADC) comprising an anti-CDCP1 antibody or antigen-binding fragment thereof conjugated to at least one drug, wherein the antibody or antigen-binding fragment thereof comprises a heavy chain variable (VH) region sequence set forth in SEQ ID NO: 2 and/or light chain variable (VL) region sequence set forth in SEQ ID NO:3. In one example, the anti-CDCP1 antibody comprises heavy chain variable and constant region sequences set forth in SEQ ID NO:12 and/or light chain variable and constant region sequences set forth in SEQ ID NO:13. In one example, the drug is a therapeutic agent. In another example, the therapeutic agent of the ADC is a cytotoxic or cytostatic agent. In one example, the cytotoxic or cytostatic agent in the ADC is a microtubule inhibitor or a DNA alkylator. In one example, the cytotoxic or cytostatic agent in the ADC is selected from the group consisting of DM4, MMAE, PDX, PDB, and IGN. In one example, the agent is MMAE. In one example, the therapeutic agent is a radionuclide. In one example, the antibody or antigen-binding fragment in the antibody drug conjugate is linked to the therapeutic agent by a linker. In one example, the linker in the ADC is selected from the group consisting of a cleavable peptide, a charged hindered disulfide, and maleimido-caproyl-valine-citrulline. In one example, the therapeutic agent in the ADC is MMAE. In a further example, the linker is (Gly)nS, wherein n is a number from about 4 to about 20, more preferably GGGGS.

In a thirteenth aspect, the disclosure provides an antibody drug conjugate having the formula Ab-[L-D]n, wherein Ab comprises the antibody or antigen-binding fragment thereof described herein, wherein L comprises an optional linker; D is a therapeutic agent; and n is an integer from about 1 to about 20.

In a fourteenth aspect, the disclosure provides a pharmaceutical composition comprising the anti-CDCP1 antibody or antigen-binding fragment thereof as described herein together with a pharmaceutically acceptable carrier. The composition of the present disclosure may be administered alone or in combination with other treatments, therapeutics or agents, either simultaneously or sequentially.

In a fifteenth aspect, the disclosure provides a pharmaceutical composition comprising an ADC mixture comprising a plurality of the ADCs as described herein, and a pharmaceutically acceptable carrier. In one example, the ADC mixture has an average drug to antibody ratio (DAR) of 0 to 8, preferably 4 to 5.

In a sixteenth aspect, the disclosure provides a method for detecting a CDCP1-expressing cancer cell in vitro or in vivo, the method comprising contacting the cancer cell in a subject or in a biological sample obtained from the subject with the anti-CDCP1 antibody or antigen-binding fragment thereof described herein. In one example, the sample is a biopsy sample. In another example, the biological sample is selected from tissue, blood, urine, saliva or other bodily fluid.

In one example, the method further comprises detecting binding of the anti-CDCP1 antibody or antigen-binding fragment thereof to the cancer cell or sample, thereby detecting CDCP1. In one example, the method further comprises recording the detection or non-detection of CDCP1 in the clinical records of a subject from whom the sample was obtained. In some examples, the clinical record is stored on a tangible computer readable medium, e.g., a disc, magnetic tape, or computer memory.

In some examples, the antibodies described herein are contacted with a biological sample and/or a cell, and the antibody to CDCP1 can be used in an immunoassay (e.g., an enzyme-linked immunosorbent assay), fluorescence-assisted cell sorting, microfluidics, or chromatography.

In some examples, the antibody is linked (e.g., covalently bonded, hydrogen bonded, or ionically bonded) to a surface (e.g., a microfluidic device, a chromatography resin, an array, polymer, or a bead).

In a seventeenth aspect, the disclosure provides a method for treating a CDCP1 expressing cancer, comprising administering a therapeutically effective amount of the antibody or antigen binding fragment thereof, labelled anti-CDCP1 antibody or antigen-binding fragment thereof, ADC, nucleic acid or composition described herein, to a subject in need thereof. In one example, the cancer is selected from the group consisting of breast cancer, lung cancer, small cell lung cancer, liver cancer, pancreatic cancer, ovarian cancer, kidney cancer, and colon cancer. In one example, the cancer is ovarian cancer. In another example, the cancer is pancreatic cancer.

In an eighteenth aspect, the disclosure provides a method for inhibiting or decreasing solid tumour growth in a subject having a solid tumour, the method comprising administering a therapeutically effective amount of the antibody or antigen-binding fragment thereof, labelled anti-CDCP1 antibody or antigen-binding fragment thereof, ADC, nucleic acid or composition described herein to the subject having the solid tumour, such that the solid tumour growth is inhibited or decreased.

In one example, the tumour is characterized as having CDCP1 expression or overexpression.

In one example, the anti-CDCP1 antibody or antigen binding fragment is administered to the subject prophylactically.

In one example, the anti-CDCP1 antibody or antigen binding fragment thereof, labelled anti-CDCP1 antibody or antigen-binding fragment thereof, ADC, nucleic acid or composition described herein is administered to the subject in a therapeutically effective amount.

Preferably, the subject is a human.

In some examples, the antibody or antigen binding fragment thereof, labelled anti-CDCP1 antibody or antigen-binding fragment thereof, ADC, nucleic acid or composition described herein is administered in combination with an additional agent or an additional therapy. In one embodiment, the additional agent is an immune checkpoint inhibitor, e.g., an antibody, such as an antibody selected from the group consisting of an anti-PD1 antibody, an anti-PD-L1 antibody and an anti-CTLA-4 antibody. In one embodiment, the additional therapy is radiation. In one example the additional agent is a chemotherapeutic agent.

In one example, the labelled antibody comprises the anti-CDCP1 antibody or antigen-binding fragment thereof conjugated to a radionuclide.

In an nineteenth aspect, the disclosure provides for use of the anti-CDCP1 antibody or antigen-binding fragment thereof, labelled anti-CDCP1 antibody or antigen-binding fragment thereof, ADC, nucleic acid or composition described herein in medicine.

In a twentieth aspect, the disclosure provides for use of the anti-CDCP1 antibody or antigen binding fragment thereof, labelled anti-CDCP1 antibody or antigen-binding fragment thereof, ADC, nucleic acid or composition described herein of the present disclosure in the manufacture of a medicament for the treatment or prophylaxis of cancer.

The disclosure features the antibodies, nucleic acids, compositions, and cells disclosed herein and the use thereof for treatment, prophylaxis, imaging, and/or diagnosis of a cancer. In some examples, the composition is formulated for intravenous administration. In some examples, the cancer expresses (e.g., overexpresses) CDCP1 or has CDCP1 on the cell surface. In some examples, the cancer is characterized by the presence of a CDCP1 and/or an elevated level of a CDCP1 protein (e.g., as compared to a reference level, e.g., a level of a CDCP1 protein in a CDCP1 protein produced by a healthy subject) produced by the cancer cells.

In one example, the subject in need thereof is a subject having, or identified or diagnosed as having a cancer characterized by overexpression of CDCP1 in cancer cells, or a cancer characterized by having CDCP1 on the surface of the cancer cells.

In some examples, the subject is identified as being a subject who expresses CDCP1, e.g., using any of the methods described herein, or has an elevated level of a CDCP1 protein, e.g., as compared to a reference level, e.g., a level of a CDCP1 protein in a CDCP1 protein produced by a healthy subject, a level of a CDCP1 protein in CDCP1 protein produced by a non-cancerous, e.g., primary cell, or a threshold level of a CDCP1 protein, in which a determined level of a CDCP1 protein that is above this value indicates that the subject should be administered an antibody described herein.

In a twenty first aspect, the disclosure provides methods for cancer prophylaxis (or reducing a subject's risk of developing a cancer characterized by expression, e.g., overexpression, of CDCP1 protein in cancer cells or a cancer characterized by having CDCP1 on the surface of the cancer cells) in a subject in need thereof comprising administering an anti-CDCP1 antibody or antigen-binding fragment thereof, labelled anti-CDCP1 antibody or antigen-binding fragment thereof, ADC, nucleic acid, or composition disclosed herein to a subject in need thereof in a prophylactically effective amount. In some examples, the cancer expresses CDCP1 protein. In some examples, the cancer cells have CDCP1 on the cell surface. In some examples of any of these methods, the subject is identified as having an elevated risk of developing cancer.

In a twenty second aspect, the disclosure provides a cancer theranostic agent, comprising the anti-CDCP1 antibody or antigen-binding fragment thereof described herein coupled to a radionuclide. In one example, the radionuclide is a diagnostic radionuclide. In another example, the diagnostic radionuclide is selected from one or more from the group consisting of ¹¹C, ¹⁸F, ⁶¹Cu, ⁶⁴Cu, ⁶⁸Ga, 86Y, ⁸⁹Zr, ¹²⁴I, ¹¹¹In, ²⁰³Pb. In one example, the radionuclide is a therapeutic radionuclide. In another example, the therapeutic radionuclide is selected from one or more from the group consisting of ²¹³Bi, ¹⁶⁶Ho, ¹³¹I, ²¹²Pb, ¹⁷⁷Lu, ²²³Ra, ¹⁸⁶Re, ¹⁵³Sm, ⁸⁹Sr, ²²⁷Th, 90Y, ²²⁵Ac, ²¹¹At, and ⁶⁷Cu.

In a twenty third aspect, the disclosure provides an immunotherapeutic agent comprising the anti-CDCP1 antibody or antigen binding fragment thereof described herein, wherein the immunotherapeutic agent comprises an immune cell modified to contain a chimeric antigen receptor (CAR). In one example, the immune cell is a T cell. In another example the immune cell is selected from one or more of NK cells, iNKT cells, and γδ T cells.

In a twenty fourth aspect, the disclosure provides for the use of the anti-CDCP1 or antigen binding fragment thereof as an imaging agent. In one example, the anti-CDCP1 or antigen binding fragment thereof is used as a PET imaging agent.

### BRIEF DESCRIPTION OF THE DRAWINGS

FIG. 1 shows antibody-induced loss of CDCP1 from the cell surface. (A) Western blot analysis of lysates from the indicated cell lines using three anti-CDCP1 antibodies, rabbit polyclonal 4115 and mouse monoclonal 10D7 and 41-2, and an anti-GAPDH antibody. (B) Flow cytometry analysis of the indicated cell lines for plasma membrane localized CDCP1 using 10D7 and 41-2. Fixed cells were stained with the respective anti-CDCP1 antibody followed by an APC-conjugated anti-mouse IgG, then analysed by flow cytometry. Data are displayed graphically as MFI values corrected for background signal determined from cells stained with only the APC-conjugated anti-mouse IgG. (C, D) Flow cytometry analysis of HEY (C) and OVMZ6-CDCP1 (D) cells treated for 30 minutes at 37° C. with 10D7, 41-2 or control IgG1κ (5 μg/ml). Treated cells were fixed and plasma membrane localized CDCP1 detected using fluorescently tagged anti-CDCP1 antibody CD318-PE. Background signal was assessed by staining treated cells with fluorescently tagged control IgG (IgG-PE). Data are displayed as MFI values. All data are mean±SEM from three independent experiments. **P<0.01; ***P<0.001.

FIG. 2 Degradation of CDCP1 induced by internalizing mAbs 41-2 and 10D7. (A) Western blot analysis, using anti-CDCP1 antibody 4115 and an anti-GAPDH antibody, of lysates from HEY cells treated with 10D7 (left) or 41-2 (right) for the indicated times. (B) Graph of fluorescence versus time from HeLa and HeLa-CDCP1 cells treated with 10D7 or 41-2 labelled with a pH sensitive fluorescent dye (10D7 ^{pH}, 41.2 ^{pH}; (5 μg/ml) (left), and graph of fluorescence signal from six EOC cell lines following treatment with 10D7 ^{pH} or 41_2 ^{pH} (5 μg/ml) for 8 hours (right). RFU, relative fluorescence units. (C) Impact of lysosomal (left) and proteasomal (right) inhibition on antibody-induced degradation of CDCP1. Top panel, Anti-CDCP1 and -GAPDH Western blot analysis of HEY cells treated with 10D7 in the presence or absence of the lysosomal inhibitor chloroquine (CLQ; 50 μM), or the proteasomal inhibitor MG132 (20 PM) for the indicated times. Bottom panel, Graph of the ratio of CDCP1 to GAPDH signal generated from Western blot analyses of lysates from three independent assays assessing the effect of CLQ and MG132 on 10D7-induced degradation of CDCP1. All data represent mean±SEM from three independent experiments. *P<0.05; **P<0.01.

FIG. 3 shows 10D7 and 41-2 bind with high affinity to the ECD of CDCP1. (A) Schematic representation of full length CDCP1 (CDCP1 FL) and progressively shorter carboxyl terminal truncations (CDCP1-T358, -S416, -K554, -D665). (B) 10D7 and 41-2 Western blot analysis of conditioned media from OVMZ6 cells transiently transfected with a control vector of constructs encoding CDCP1-T358, -S416, -K554, or -D665. (c) Top panels, Sensorgrams of CDCP1-ECD (concentration range 1.56 to 50 nM) binding to immobilized 10D7 (left) and 41-2 (right) depicting association (increasing signal) and dissociation (reducing signal) over time. Bottom panel, Table of kinetic parameters. kₐ, association rate; k_{d}, dissociation rate; K_{D}, affinity constant.

FIG. 4 shows 10D7-induces rapid cell surface clustering and lysosomal trafficking of CDCP1. (A) Live-cell confocal microscopy images of HEY-CDCP1^{GFP} cells treated with 10D7 pH (5 μg/ml). Internalization of CDCP1^{GFP} and 10D7^{pH} was observed at 1 frame per second for 600 s. Insets highlight green punctate CDCP1^{GFP} positive cellular structures at 30 s, and white cellular structures at 600 s that are positive for both CDCP1 ^{GFP} and 10D7 ^{pH}. (B) graph of complex formation between IgG^{pH} and CDCP1 ^{GFP} determined as the percentage of IgG^{PH} signal coincident with CDCP1^{GFP} signal using ImageJ software analysis, (C) Images of the plasma membrane and proximal cytoplasmic region of HEY-CDCP1^{GFP} cells indicating 10D7-induced clustering of CDCP1. In untreated cells CDCP1 ^{GFP} is located diffusely on the cell surface. In treated cells, arrowheads highlight rapid 10D7-induced clustering of CDCP1^{GFP} and its internalization. (D) Left panel, Overlay of CDCP1^{GFP} (green) and 10D7^{pH} (purple) signals in HEY-CDCP1^{GFP} cells after 20 minutes of treatment showing co-localization of within endosomal-like structures. Middle panel, Black and white image of CDCP1 ^{GFP} signal. Right panel, Black and white image of 10D7 ^{pH} signal. (d) Live-cell confocal microscopy images of HEY-CDCP1^{GFP} cells treated with IgG7^{pH} (5 μg/ml). No internalization of CDCP1 ^{GFP} was observed within 300 s of treatment with IgG7 ^{pH}.

FIG. 5 shows CDCP1 is tyrosine phosphorylated during 10D7-induced internalization and degradation. (A) Lysates from HEY cells treated with 10D7 (5 μg/ml) for the indicated times were examined by Western blot analysis for CDCP1, p-CDCP1-Y734, Src, p-Src-Y416, and GAPDH. The graphs display CDCP1 and p-CDCP1-Y734 levels determined by densitometric analysis with data representing mean±SEM from three independent experiments. (B) Anti-CDCP1, -GFP and -GAPDH Western blot analysis of fractions collected by cell surface biotinylation of HEY cells expressing CDCP1^{GFP}, CDCP1^{GFP}-Y734F, -Y743F or -Y762F. (C) Analysis of semi-automated computer tracking of CDCP1 ^{GFP} and CDCP1^{GFP}-Y734F, -Y743F and -Y762F in response to 10D7. Left, representative image of CDCP1 ^{GFP} tracks that internalized in response to 10D7 ^{pH} in HEY cells. The image is an overlay onto cells of color-coded tracks (violet, tracks that moved the shortest distance; red, the tracks that moved the greatest distance). Right, Graph of distance moved over 5 min by CDCP1 ^{GFP} and CDCP1^{GFP}-Y734F, -Y743F and -Y762F in response to 10D7 (5 μg/ml). Data are median and range from the 100 tracks with the highest velocity in each experimental group from three independent experiments. ***P<0.001.

FIG. 6 shows the Src inhibitor dasatinib blocks 10D7-induced phosphorylation and internalization of CDCP1. (A) HEY cells, treated for 2 h with dasatinib (200 nM), were incubated with 10D7 (5 μg/ml) for the indicated times. Lysates were examined by Western blot analysis for CDCP1, p CDCP1-Y734 and GAPDH. (B) Live-cell confocal microscopy images, acquired at the indicated time points after antibody treatment, of HEY-CDCP1^{GFP} cells pre-treated with dasatinib (200 nM), then incubated with 10D7 ^{pH}. Lower panels, 10D7^{pH} signal. Middle panels, CDCP1^{GFP} signal. Upper panels, overlay of 10D7 ^{pH} and CDCP1^{GFP} signals. (C) Graph of distance moved over 5 min by CDCP1 ^{GFP} in response to 10D7 in the presence and absence of dasatinib. Data are median and range from the 100 tracks with the highest velocity in each experimental group from three independent experiments. ***P<0.001.

FIG. 7 shows PET-CT imaging of an EOC PDX. (A) Clear cell EOC PDX PH250. Left, hematoxylin and eosin stained section highlighting clear cell features at 40× with 10× magnification (inset). Right, Anti-CDCP1 immunohistochemistry (antibody 4115) highlighting strong CDCP1 expression by malignant cells with accentuation of signal on the plasma membrane at 40× and 1 OX magnification (inset). (B) Comparison of CDCP1 expression by HEY cells and PDX PH250 cells. Left Anti-CDCP1 (antibody 4115; 1:2,000) and GAPDH (1:10,000) Western Blot analysis of lysates from HEY cells (a HEY cell xenograft, and a PH250 PDX tumour) Right Cell Surface CDCP1 receptor number determined by flow cytometry of single cell suspensions of HEY cells and PDX PH250 cells. Receptor numbers per cell are indicated above the flow cytometry peaks. (C) Representative PET images of mice 3 weeks post-inoculation of PDX PH250 bearing intraperitoneal tumours, at 48 and 144 h post injection (P.I.) of ⁸⁹Zr-DFO-10D7 and ⁸⁹Zr-DFO-IgG1K. 10D7 accumulates in tumours whereas IgG1K does not. The chemical yield of ⁸⁹Zr-DFO-10D7 and ⁸⁹Zr-DFO-IgG1κ were 81% and 78% respectively, with purity of >95%. (D) Quantitative bio-distribution analysis of ⁸⁹Zr-DFO-10D7 and ⁸⁹Zr-DFO-IgG1κ 144 h post injection (n=4). 10D7 accumulates in tumours to a significantly higher degree than IgG1κ (P=0.017) which accumulates in the spleen and liver.

FIG. 8 shows 10D7-MMAE selectively inhibits colony formation of CDCP1 expressing but not non-expressing EOC cells. (A) HEY cells were treated with 10D7-MMAE (5 μg/ml) for the indicated times and lysates examined by Western blot analysis for CDCP1, p-CDCP1-Y734, Src, p-Src-Y416 and GAPDH. (B) Representative images of crystal violet stained colonies formed from HEY and OVMZ6-CDCP1 cells after treatment with the indicated concentrations of IgG, IgG-MMAE, 10D7 or 10D7-MMAE. (C) Graph of crystal violet staining, as a percentage of area (% Area), of colonies formed by HEY, OVMZ6-CDCP1 and OVMZ6 cells after treatment with increasing concentrations of IgG, IgG-MMAE, 10D7 or 10D7-MMAE. Data represent means±SEM from three independent experiments. **P<0.01, ***P<0.001.

FIGS. 9-1 to 9-3 shows CDCP1 expression in PDAC tumours and processing in PDAC cells. (A), Kaplan-Meier analysis showing correlation between CDCP1 mRNA expression levels and PDAC patient survival in TCGA-GDC (n=170) and ICGC PACA-AU (n=220) datasets. Patients in each dataset with CDCP1 mRNA expression levels in the first and fourth quartile were segregated into low and high CDCP1 expressing groups, respectively. (B), Kaplan-Meier analysis showing correlation between CDCP1 protein expression levels and PDAC patient survival in the ICGC PACA-AU (n=223) cohort. For this analysis patients with CDCP1 expression at or below the average score were segregated into “low” and those with expression above the average were segregated into the “high” CDCP1 expressing group. Statistical differences between Kaplan-Meier curves were determined by Mantel-Cox test. (C), Diagram depicting structural features of CDCP1 including three extracellular CUB-like domains (grey rectangles), proteolytic processing sites at R368 and K369, amino-terminal (ATF) and carboxyl-terminal (CTF) CDCP1 fragments, and binding sites of the anti-CDCP1 antibodies 10D7, 4115 and 2666. (D), Western blot analysis under reducing conditions of nine patient-derived PDAC cells (TKCC) and two PDAC cell lines using anti-CDCP1 antibodies 4115 and 2666, and an anti-GAPDH antibody. Graph, quantified from densitometry analysis of western blots of three independent lysate preparations of each cell. (E), Flow cytometry analysis of PANC-1, TKCC02, TKCC05 and TKCC10 cells for plasma membrane CDCP1 using antibody 10D7. (F), Anti-CDCP1 western blot analysis with the indicated antibodies of proteins immunoprecipitated with antibody 10D7 or isotype matched control antibodies. (G), Analysis of fractions collected by chromatography of untreated or trypsin treated CDCP1 extracellular domain (ECD). Top, UV/Vis spectroscopy analysis of size-exclusion chromatography. Bottom, Coomassie stained gel analysis of fractions collected during chromatography.

FIGS. 10-1 to 10-2 shows expression and processing of CDCP1 in PDAC cells in vitro and immunohistological analysis of mouse xenografts of patient-derived PDAC cells. (A), Western blot analysis using anti-CDCP1 antibodies 4115 and 2666, and an anti-GAPDH antibody, of PANC-1, TKCC02, TKCC05 and TKCC10 cell lysates under reduced condition before and after enzymatic deglycosylation with N-glycosidase F (PGNase F) for 1 h at 37° C. (B), Hematoxylin and eosin staining (left) and CDCP1 immunohistochemistry (right, antibody 4115) of xenografts of PANC-1, TKCC02, TKCC05 and TKCC10 PDAC cells grown subcutaneously in mice. (C). Confocal microscopy imaging of PANC-1, TKCC05 and HeLa cells after immunofluorescent staining of CDCP1 (antibody 10D7) and co-staining of nuclei and membrane with DAPI and wheat germ agglutinin-FITC (WGA), respectively. Scale bar=20 μm. (D). Western blot analysis using anti-CDCP1 antibodies 4115 and 2666, and an anti-GAPDH antibody, of PANC-1, TKCC02, TKCC05 and TKCC10 cell lysates under reducing (left) and non-reducing (right) conditions.

FIGS. 11-1 to 11-3 shows cell binding and internalization of function blocking antibody 10D7 which induces rapid phosphorylation, internalization and degradation of differentially cleaved CDCP1 in PDAC cells. (A), Confocal microscopy analysis of TKCC05 cells treated with fluorescently tagged antibody 10D7 (10D7-Qdot, red). After the indicated times cells were fixed then stained with phalloidin (green) and DAPI (blue) to highlight cell cytoplasm and nucleus, respectively (top). Specific 10D7-Qdot signal shows membrane localization then internalization of 10D7 (bottom). (B), Western blot analysis of lysates from TKCC05 (left) and TKCC10 (right) cells treated for up to 300 min with antibody 10D7 or isotype matched IgG. Lysates were probed for p-CDCP-Y734, CDCP1 (antibody 4115), p-Src-Y417, Src and β-actin. Graphs quantify changes in levels of p-CDCP1-Y734, CDCP1 and p-Src in response to 10D7. (C), Western blot analysis of lysates from TKCC05 (left) and TKCC10 (right) cells treated for longer periods with 10D7 or isotype matched IgG. Lysates were probed for CDCP1 (antibody 4115), β-actin and mouse IgG (heavy and light chains). Graphs quantify changes in levels of CDCP1 in response to 10D7. (D), Western blot analysis of lysates collected from TKCC05 cells treated for 48 h with antibody 10D7 or isotype matched IgG before antibody washout then further growth up to 72 h in normal medium. Lysates were probed for CDCP1 (antibody 4115), β-actin and mouse IgG. Graphs quantify changes in levels of CDCP1 in response to 10D7. FL: Full length; CTF; carboxyl-terminal fragment; ATF: amino-terminal fragment.

FIG. 12 shows function blocking antibody 10D7 induces rapid phosphorylation and degradation of differentially cleaved CDCP1 in PDAC cells. (A). Western blot analysis of lysates of PANC-1 (left) and TKCC02 (right) cells treated for up to 300 min with anti-CDCP1 antibody 10D7 or isotype matched IgG. Lysates were probed for p-CDCP-Y734, CDCP1 (antibody 4115), p-Src-Y417, Src and β-actin. (B), Western blot analysis of lysates of PANC-1 (left) and TKCC02 (right) cells treated for longer periods with anti-CDCP1 antibody 10D7 or isotype matched IgG. Lysates were probed for CDCP1 (antibody 4115) and β-actin.

FIGS. 13-1 to 13-2 shows functional blockade of CDCP1 reduces cell migration and non-adherent spheroid growth, and improves chemo-responsiveness of PDAC cells in vitro. (A), Transwell migration assay was performed on transduced cells (2.5×10⁵/well) stably expressing control shRNA, CDCP1 shRNA (two constructs) or with parental cells treated with 10D7 (5 μg/ml), isotype matched IgG (5 μg/ml) or PBS. Relative migration was determined by measurement of absorbance at 590 nm of crystal violet that was methanol extracted from stained cells. (B), Relative spheroid growth was quantified 10 days after cell suspensions (10,000/200 μl/well, same condition as above) were plated in 96-well ultra-low attachment plates in serum free, growth factor restricted media. Quantification was performed by absorbance measurements at 490 nm of wells incubated with the CellTiter AQueous One Solution Reagent. (C), Left: Survival analysis was performed on transduced cells stably expressing control shRNA or CDCP1 shRNA or with parental cells pre-treated with 10D7 (5 μg/ml) or isotype matched IgG (5 μg/ml) for 24 h before treatment with gemcitabine (0.02 to 500 nM) for 72 h. Relative cell survival was then determined by absorbance measurements at 490 nm of wells incubated with the CellTiter AQueous One Solution Reagent. Right: Western blot analysis of lysates collected from transduced cells stably expressing control shRNA or CDCP1 shRNA (construct #1) or from parental cells pre-treated with 10D7 (5 μg/ml), isotype matched IgG (5 μg/ml) treated for 24 h before treatment with 10D7 or IgG (5 μg/ml) in the presence of gemcitabine (at two concentrations close to the G150 of each line) for 48 h. Lysates were probed with antibodies against cleaved PARP (cPARP), CDCP1 (antibody 4115) and GAPDH. Statistical significance between different groups was assessed using the Kruskal-Wallis test with * p<0.05, ** p<0.01 and *** p<0.001.

FIG. 14 shows total and cell surface expression of CDCP1 in PDAC cells. (A). Western blot analysis of lysates of PANC-1, TKCC02, TKCC05 and TKCC10 PDAC cells stably expressing CDCP1-shRNA (shCDCP1 #1 and #2) or scramble shRNA (shRNA Control). Lysates were probed for CDCP1 (antibody 4115) and GAPDH. (B), Quantification of CDCP1 receptor number on the cell surface by flow cytometry analysis of PANC-1, TKCC02, TKCC05 and TKCC10 PDAC cells stably expressing ShCDCP1 #1 or ShRNA Control.

FIGS. 15-1 to 15-2 shows 10D7 antibody detects PDAC cells in vivo. (A), Lindmo plot showing binding of 10D7-89Zr to an increasing number of CDCP1-positive TKCC05 cells. (B), Representative PET-CT images of NSG mice carrying subcutaneous TKCC05 cell tumours. 10D7-⁸⁹Zr- and IgG1K-⁸⁹Zr were injected intravenously two weeks after tumour cell inoculation, and imaging performed 24, 48, 72 and 144 h later. White arrow, tumour nodules. (C), Quantitative bio-distribution analysis of 10D7-⁸⁹Zr and IgG1K-⁸⁹Zr 144 h post injection (n=3). (D), Left: Bioluminescence imaging (top) and PET-CT imaging with 10D7-⁸⁹Zr as the contrast agent (bottom) of TKCC05-shCDCP1 and TKCC05-shControl cell xenografts. Right: Quantitative distribution analysis of 10D7-⁸⁹Zr and IgG1K-⁸⁹Zr 144 h post injection (n=3) in TKCC05-shCDCP1 compared with TKCC05-shControl cell xenografts. Statistical significance between different groups was performed using a two-way ANOVA test with *** p<0.001.

FIGS. 16-1 to 16-2 shows functional targeting of CDCP1 reduces tumour burden and improves gemcitabine efficacy in vivo. (A). Effect of antibody targeting of CDCP1 on PANC-1 cell xenograft growth. Top, Two weeks after subcutaneous inoculation of PANC-1 cells (average tumour size ˜100 mm3) mice (6/group) were randomized and treated i.v. twice a week with PBS, 10D7 (5 mg/kg) or IgG (5 mg/kg). Middle left, Graph of tumour volume measured weekly by calliper. Middle right, Graph of tumour weight at experimental end-point after 9 weeks of growth. Bottom, Western blot analysis of lysates collected from representative PANC-1 cell xenografts. Antibodies were against CDCP1 (4115), mouse IgG and GAPDH. (B), Effect of antibody targeting of CDCP1 on TKCC05 cell xenograft growth in combination with gemcitabine chemotherapy. Top, Three weeks after subcutaneous inoculations of TKCC05 cells, mice were randomized and treated i.v. twice a week with PBS (n=12), 10D7 (n=12, 5 mg/kg) or IgG (n=12, 5 mg/kg). Half of the mice in each group also received gemcitabine i.p. treatments (125 mg/kg weekly the day after antibody treatment). Middle left, Graph of tumour volume measured weekly by calliper. Middle right, Graph of tumour weight at end-point. Bottom, Representative anti-CDCP1 (antibody 4115) stained sections from recovered TKCC05 cell xenografts. (C and D), Impact of CDCP1 silencing on growth of subcutaneous xenografts of PANC-1 (C) and TKCC05 (D) cells stably expressing ShRNA control (ShControl) or ShRNA CDCP1 (ShCDCP1). Top left, Graph of tumour volume measured weekly by calliper. Top right, Graph of tumour weight at end-point. Bottom, Representative images of immunohistochemical analysis of CDCP1 expression (antibody 4115) in xenografts. Statistical significance has been determined by Mann-Whitney test between indicated groups with * p<0.05, ** p<0.01 and *** p<0.001.

FIG. 17 shows CDCP1 expression in PDAC mouse xenografts. (A), Immunohistochemical staining for CDCP1 (antibody 4115) of representative PANC-1 xenograft tumours from mice treated with PBS, IgG or 10D7. (B and C). Western blot analysis of representative lysates from subcutaneous xenografts from PANC-1 (B) and TKCC05 (C) cells stably expressing control ShRNA (ShControl) or CDCP1 ShRNA (ShCDCP1). Lysates were probed for CDCP1 (antibody 4115) and GAPDH. (C), Representative images from transwell migration assays at end point (48 h) performed with PANC-1, TKCC02, TKCC05 and TKCC10 PDAC cells stably expressing CDCP1-shRNA (ShCDCP1 #1 and #2) or scramble shRNA (ShRNA Control) or treated with 10D7 (5 μg/ml), isotype matched IgG (5 μg/ml) or PBS. Scale bar=200 am. (D), Survival analysis was performed on transduced PANC-1 and TKCC02 cells stably expressing control ShRNA or CDCP1 ShRNA treated with gemcitabine (0.01 to 50 μM) for 72 h. Relative cell survival was then determined by absorbance measurements at 490 nm of wells incubated with the CellTiter AQueous One Solution Reagent.

FIGS. 18-1 to 18-2 shows antibody 10D7 is effective for specific cytotoxin delivery to PDAC cells in vitro and in vivo. (A), Relative survival of PANC-1, TKCC02, TKCC05 and TKCC10 cells treated for 12 h with 10D7-MMAE or IgG-MMAE (0.0625, 0.125, 0.25, 0.5 and 1 ag/ml) or 10D7 or IgG (0.5 and 1 μg/ml) then grown for a further 72 h in complete medium. Quantification was performed by absorbance measurements at 490 nm of wells incubated with the CellTiter AQueous One Solution Reagent. (B), Left, TKCC05 cells expressing monomeric Kusabira-Orange 2 (mKO2; red) co-cultured with GFP-expressing normal human pancreatic stellate cells (hPSC; green) treated as above. Right, Graph of survival of TKCC05 and hPSC cells quantified from the confluency area for each cell type from fluorescent microscopy images from the red and green channels, respectively. (C), Effect on growth of antibody-mediated cytotoxin delivery to CDCP1 expressed by subcutaneous TKCC05 cell xenograft. Top, Day 27 after inoculation of TKCC05 cells, mice (6/group) were randomized and treated on that day and day 41 i.v. with PBS, 10D7 (5 mg/kg), IgG (5 mg/kg), 10D7-MMAE (5 mg/kg) or, IgG-MMAE (5 mg/kg), or on day 27, 34, 41 and 48 with i.p. gemcitabine (125 mg/kg). Bottom, Graph of tumor volume measured weekly using callipers until day 49 when the first mice in the control groups required euthanasia due to disease burden. (D), Kaplan-Meier survival curve of mice in each treatment group from D. Statistical significance in comparison to control group (PBS) was determined by the Kruskal-Wallis test with * p<0.05, ** p<0.01 and *** p<0.001. The Mann-Whitney test has been used when two-groups are compared (C and E).

FIG. 19 shows flow cytometry analysis of binding to TKCC05 pancreatic cancer cells expressing CDCP1. Binding for the 10D7 mouse antibody is shown left and the human/mouse chimeric 10D7 antibody and corresponding Fab is shown right.

FIG. 20 shows that the human/mouse chimeric 10D7 antibody is able to detect CDCP1 expressing TKCC02 pancreatic cancer subcutaneous xenograft tumours in vivo (upper panel) and accumulates in the tumour to a higher degree than the mouse 10D7 antibody (lower panel).

FIG. 21 shows that MMAE labelled antibody 10D7 and MMAE labelled human/mouse chimeric 10D7 antibody are effective at inhibiting proliferation of TKCC05 pancreatic cancer cells as measured in real-time (A) and at the end point of the assay. (A) Cell confluence over time (relative to control cells treated with vehicle) measured using an Incucyte S3 instrument. (B) Cell confluence at end point (96 h of treatment) expressed as confluence (%) relative to control cells treated with vehicle as measured by plate reader by absorbance measurement at 490 nm.

FIG. 22 shows by flow cytometry analysis that antibody 10D7 and human/mouse chimeric 10D7 antibody bind to CDCP1 expressing TKCC05 pancreatic cancer cells and HEY ovarian cancer cells (A), and that these antibodies compete for binding to CDCP1 expressing cells (B) and that saturating levels of each antibody blocks the other antibody binding to CDCP1 expressing cells (C), indicating that binding of the Complementarity-Determining Regions of 10D7 are retained in the human/mouse chimeric 10D7 antibody.

FIG. 23 shows by Western Blot analysis (with anti-CDCP1 4115 antibody) of protein lysates from HEY ovarian cancer cells that antibody 10D7 and the human/mouse chimeric 10D7 antibody are internalised and induce the degradation of CDCP1, indicating their functional equivalence in this assay format in vitro.

FIG. 24 shows PET/CT imaging of mice carrying intra-pancreatic TKCC05 pancreas cancer tumour using antibody 10D7 and the human/mouse chimeric 10D7 antibody labelled with Zircodium-89. (A) PET-CT imaging of mice detects intra-pancreatic tumours. (B) shows bio distribution in harvested organs; and (C) shows correlation analysis between Zr89 accumulation in pancreas and bioluminescence signal from the pancreas.

FIG. 25 shows (A) tumour burden and (B) survival in mice that were grafted subcutaneously with TKCC05 pancreatic cancer cells. Four weeks after injection of TKCC05 cells mice were randomised then treated twice with 10D7-MMAE, human/mouse-10D7-MMAE or control IgG-MMAE, or three times with gemcitabine chemotherapy, or untreated. The data indicate 10D7-MMAE and human/mouse-10D7-MMAE slow tumour growth and prolong mouse survival more effectively than standard of care chemotherapy and that IgG-MMAE was ineffective.

FIG. 26 shows internalisation of antibody 10D7 and human/mouse chimeric 10D7 antibody in pancreatic cancer cells measured in vitro. TKCC05 cells stably silenced for CDCP1 expression using an ShRNA-targeting CDCP1 or control ShRNA TKCC05 cells in 96 well/plate format were treated with 2 μg/ml of IgG isotype control, 10D7 or huma/mouse-10D7 pre-labelled with a pH-sensitive fluorescent dye (FabFluor-pH Red Antibody Labeling Reagent, Essen-Biosciences). Fluorescence per cell was measured in real time using an Incucyte S3 over 8 h with scanning every 15 min, and demonstrated that antibody 10D7 and human/mouse chimeric 10D7 antibody are internalised specifically via CDCP1.

### KEY TO SEQUENCE LISTING

SEQ ID NO:1: amino acid sequence of isoform 1 of human CDCP1 (UniProtKB Reference Q9H5V8) including leader sequence
SEQ ID NO:2: amino acid sequence of VH of antibody 10D7
SEQ ID NO:3: amino acid sequence of VL of antibody 10D7
SEQ ID NO:4: sequence of VH CDR1 of antibody 10D7
SEQ ID NO:5: sequence of VH CDR2 of antibody 10D7
SEQ ID NO:6: sequence of VH CDR3 of antibody 10D7
SEQ ID NO:7: sequence of VL CDR1 of antibody 10D7
SEQ ID NO:8: sequence of VL CDR2 of antibody 10D7
SEQ ID NO:9: sequence of VL CDR3 of antibody 10D7
SEQ ID NO:10: amino acid sequence of human heavy chain constant region
SEQ ID NO:11: amino acid sequence of human light chain constant region
SEQ ID NO:12: amino acid sequence of complete heavy chain of chimeric 10D7
SEQ ID NO:13: amino acid sequence of complete light chain of chimeric 10D7
SEQ ID NO:14: nucleic acid sequence of VH of antibody 10D7
SEQ ID NO:15: nucleic acid sequence of VL of antibody 10D7
SEQ ID NO:16: control shRNA
SEQ ID NO:17: CDCP1 shRNA

### DETAILED DESCRIPTION OF THE INVENTION

### General Techniques and Selected Definitions

The term “and/or”, e.g., “X and/or Y” shall be understood to mean either “X and Y” or “X or Y” and shall be taken to provide explicit support for both meanings or for either meaning.

As used herein, the terms “a”, “an” and “the” include both singular and plural aspects, unless the context clearly indicates otherwise.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example of the disclosure unless specifically stated otherwise.

Those skilled in the art will appreciate that the disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure.

The present disclosure is performed without undue experimentation using, unless otherwise indicated, conventional techniques of molecular biology, recombinant DNA technology, cell biology and immunology. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp35-81; Sproat et a, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series, Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wunsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

The term “consists of” or “consisting of” shall be understood to mean that a method, process or composition of matter has the recited steps and/or components and no additional steps or components.

The term “about”, as used herein when referring to a measurable value such as an amount of weight, time, dose, etc. is meant to encompass variations of ±20% or ±10%, more preferably ±5%, even more preferably ±1%, and still more preferably ±0.1% from the specified amount, as such variations are appropriate to perform the disclosed method.

The description and definitions of variable regions and parts thereof, immunoglobulins, antibodies and fragments thereof herein may be further clarified by the discussion in Kabat, 1987 and/or 1991, Bork et al., 1994 and/or Chothia and Lesk, 1987 and/or 1989 or Al-Lazikani et al., 1997 or the IMGT numbering of Lefranc M.-P., (1997) Immunology 5 Today 18, 509.

It will be understood that the anti-CDCP1 antibodies and antigen-binding fragments thereof, labelled anti-CDCP1 antibodies and antigen-binding fragments thereof, nucleic acids, cells and vectors described herein are in isolated form. By “isolated” it is meant a polypeptide, antibody, polynucleotide, vector, or cell, that is in a form not found in nature. Isolated polypeptides, antibodies, polynucleotides, vectors, or cells include those which have been purified to a degree that they are no longer in a form in which they are found in nature. In some aspects, an antibody, polynucleotide, vector, or cell that is isolated is substantially pure. In some aspects an antibody, polynucleotide, vector, or cell that is isolated is “recombinant.”

As used herein, the term “biological sample” refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the subject, but the term can also refer to non-cellular biological material, such as non-cellular fractions of blood, saliva, or urine. Biological samples include, but are not limited to, tissue biopsies, needle biopsies, scrapes (e.g. buccal scrapes), whole blood, plasma, serum, lymph, bone marrow, urine, saliva, sputum, cell culture, pleural fluid, pericardial fluid, ascitic fluid or cerebrospinal fluid. Biological samples also include tissue biopsies and cell cultures. Samples may be paraffin-embedded or frozen tissue.

The term “coupled to” as used herein is intended to encompass any construction whereby the anti-CDCP1 antibody or antigen-binding fragment thereof is linked, attached or joined to a moiety as described herein. Methods for effecting coupling will be known to persons skilled in the art and include, but are not limited to conjugation, linking via peptide linker or by direct chemical synthesis.

The term “antibody” describes an immunoglobulin whether natural or partly or wholly synthetically produced or recombinantly produced. The term also covers any polypeptide or protein having a binding domain which is, or is homologous to, an antibody binding domain. CDR grafted antibodies are also contemplated by this term. An “antibody” is any immunoglobulin, including antibodies and fragments thereof, that binds a specific epitope. The term encompasses polyclonal, monoclonal, multivalent antibodies, multispecific antibodies, chimeric antibodies, humanized antibodies, and human antibodies. The term “antibody” also refers to a protein comprising at least two immunoglobulin heavy (H) chains and two immunoglobulin light (L) chains inter-connected by disulfide bonds, or an antigen binding portion thereof. Each heavy chain is comprised of a heavy chain variable region (abbreviated herein as VH) and a heavy chain constant region (abbreviated herein as CH). The CH is normally comprised of three domains, CH1, CH2 and CH3 (IgM, e.g., has an additional constant region domain, CH4). Each light chain is comprised of a light chain variable region (abbreviated herein as VL) and a light chain constant region (abbreviated herein as CL). The CL is comprised of one domain and can be of the lambda or kappa type. The VH and VL regions can be further subdivided into regions of hypervariability, termed complementarity determining regions (CDR), interspersed with regions that are more conserved, termed framework regions (FWR). Each VH and VL is composed of three CDRs and four FWRs, arranged from amino-terminus to carboxy-terminus in the following order: FWR1, CDR1, FWR2, CDR2, FWR3, CDR3, FWR4. In certain embodiments, the VH and VL together comprise a binding domain that interacts with an antigen. In other embodiments a single VH or single VL domain can interact specifically with the antigen. The CH domain of an antibody can mediate the binding of the immunoglobulin to host tissues or factors, including various cells of the immune system (e.g., effector cells), cells lining the vascular wall, other cell expressing receptors for the CH domain of immunoglobulins and the first component (C1q) of the classical complement system. Antibody molecules can be of any class (e.g., IgG, IgE, IgM, IgD, IgA, and IgY), or subclass (e.g., IgG1, IgG2, IgG3, IgG4, IgA1, and IgA2). The term “antibody” as used herein also includes “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al, Proc. Natl. Acad. Sci. USA 81:6851-6855 (1984)). Basic antibody structures in vertebrate systems are well understood. See, e.g., Harlow et al. (1988) Antibodies: A Laboratory Manual (2nd ed.; Cold Spring Harbor Laboratory Press). Also included within the meaning of the term “antibody” are any “antigen-binding fragments”.

The term “antigen-binding fragment” refers to one or more fragments of an antibody that retain the ability to specifically bind to an antigen (e.g., CDCP1). Fragments of a full-length antibody can perform the antigen-binding function of an antibody. Examples of binding fragments encompassed within the term “antigen-binding fragment” of an antibody include (i) a Fab fragment, a monovalent fragment consisting of the VL and CL, VH and CH1 domains; (ii) a F(ab)2 fragment, a bivalent fragment comprising two Fab fragments linked by a disulfide bridge at the hinge region; (iii) a Fd fragment consisting of the VH and CH1 domains; (iv) a Fv fragment consisting of the VH and CL domains of a single arm of an antibody, (v) a single domain antibody fragment or dAb (Ward et al., (1989) Nature 341:544-546), which consists of a VH domain or a VL domain only; and (vi) an isolated complementarity determining region (CDR). Furthermore, although the two domains of the Fv fragment, VH and VL, are coded for by separate genes, they can be joined, using recombinant or synthetic methods, e.g., by a synthetic linker that enables them to be made as a single protein chain in which the VH and VL regions pair to form monovalent molecules (known as single chain Fv (scFv); see e.g., Bird et al. (1988) Science 242:423-426; and Huston et al. (1988) Proc. Natl. Acad. Sci. USA 85:5879-5883). scFv are also encompassed within the term “antigen-binding fragment” of an antibody. These antibody fragments are obtained using conventional techniques known to those with skill in the art, and the fragments are screened for utility in the same manner as are intact antibodies.

As used herein, “antibody variable region” refers to the portions of the light and heavy chains of antibody molecules that include amino acid sequences of complementarity determining regions (CDRs; i.e., CDR1, CDR2 and CDR3), and framework regions (FWRs). VH refers to the variable region of the heavy chain. VL refers to the variable region of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)) or Chotia and Lesk 1987 J. Mol Biol. 196:901-917) or according to the IMGT numbering system.

The term “monoclonal antibody” as used herein refers to a preparation of antibody molecules of single molecular composition. A monoclonal antibody displays a single binding specificity and affinity for a particular epitope. The monoclonal antibodies can be generated from any animal, e.g., mouse, rat, rabbit, pig, etc., or can be generated synthetically and be in part or entirely of human sequence.

The term “chimeric antibody” refers to antibodies in which a portion of the heavy and/or light chain is identical with or homologous to corresponding sequences in antibodies derived from a particular species (e.g. murine) or belonging to a particular antibody class or subclass, while the remainder of the chain(s) is identical with or homologous to corresponding sequences in antibodies derived from another species (e.g. primate) or belonging to another antibody class or subclass, as well as fragments of such antibodies, so long as they exhibit the desired biological activity.

The term “humanized antibody” shall be understood to refer to a chimeric molecule, generally prepared using recombinant techniques, having an epitope binding site derived from an immunoglobulin from a non-human species and the remaining immunoglobulin structure of the molecule based upon the structure and/or sequence of a human immunoglobulin. The antigen-binding site preferably comprises the complementarity determining regions (CDRs) from the non-human antibody grafted onto appropriate framework regions in the variable domains of human antibodies and the remaining regions from a human antibody.

The term “human antibody” as used herein in connection with antibody molecules and binding proteins refers to antibodies having variable (e.g. VH, VL, CDR and FR regions) and constant antibody regions derived from or corresponding to sequences found in humans, e.g. in the human germline or somatic cells.

“IMGT numbering” as used herein refers to a numbering system used to identify CDR and FWR sequences of antibody variable regions. The IMGT unique numbering has been defined to compare the variable domains whatever the antigen receptor, the chain type, or the species (Lefranc M.-P., Immunology 5 Today 18, 509 (1997)/Lefranc M.-P., The Immunologist, 7, 132-136 (1999)/Lefranc, M.-P., Pommié, C., Ruiz, M., Giudicelli, V., Foulquier, E., Truong, L., ThouveninContet, V. and Lefranc, Dev. Comp. Immunol., 27, 55-77 (2003)). In the IMGT unique numbering, the conserved amino acids always have the same position, for instance cysteine 23 (1^{st} CYS), tryptophan 41 (CONSERVED-TRP), hydrophobic amino acid 89, cysteine 104 (2^{nd} CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths become crucial information. The IMGT unique numbering is used in 2D graphical representations, designated as IMGT Colliers de Perles (Ruiz, M. and Lefranc, M.-P., Immunogenetics, 53, 857-883 (2002)/Kaas, Q. and Lefranc, M.-P., Current Bioinformatics, 2, 21-30 (2007)), and in 3D structures in IMGT/3Dstructure-DB (Kaas, Q., Ruiz, M. and Lefranc, M.-P., T cell receptor and MHC structural data. Nucl. Acids. Res., 32, D208-D210 (2004)).

Reference to “CDCP1” as used herein is intended to refer to the transmembrane receptor in either its cleaved form or uncleaved form. In particular, it refers to the human receptor.

The expression “cleaved form of CCP1” as referred to herein means a CDCP1 receptor protein which is proteolytically cleaved at position 368 corresponding to arginine (R) or position 369 corresponding to lysine (K) as demonstrated by He Y et al., (2010) J Biol Chem 285(34):26162-73 generating an amino-terminal portion of CDCP1 and a membrane spanning carboxy-terminal portion of CDCP1. The inventors have previously shown that the amino terminal portion can be released from the cell surface. Studies described herein demonstrate that the amino terminal portion can also remain linked/tethered to the remainder of the receptor.

The term “anti-CDCP1 antibody”, refers to an antibody that specifically binds to CDCP1, e.g., human CDCP1. An antibody “which binds” an antigen of interest, i.e., CDCP1, is one capable of binding that antigen with sufficient affinity such that the antibody is useful in targeting a cell expressing the antigen. Preferably, the antibody specifically binds to human CDCP1 (hCDCP1). Unless otherwise indicated, the term “anti-CDCP1 antibody” is meant to refer to an antibody which binds to wild type CDCP1, a variant, or an isoform of CDCP1. The sequence of human CDCP1 is disclosed as Uniprot reference Q9H5V8 corresponding to isoform 1 (SEQ ID NO:1). The cleavage site is located at amino acid positions 368-369 of SEQ ID NO:1. Alternative splicing produces isoforms 2 lacking amino acids 1-187, and isoform 3 wherein amino acids 342-343 are modified from NK to SE and amino acids 344-836 are missing.

The terms “specific binding” or “specifically binds”, as used herein, in reference to the interaction of a CDCP1 antibody or an ADC with a second chemical species, means that the interaction is dependent upon the presence of a particular structure (e.g., an antigenic determinant or epitope) on the chemical species; for example, an antibody recognizes and binds to a specific protein structure rather than to proteins generally. If an antibody or ADC is specific for epitope “A”, the presence of a molecule containing epitope A (or free, unlabeled A), in a reaction containing labeled “A” and the antibody, will reduce the amount of labeled A bound to the antibody or ADC. In one example, the phrase “specifically binds” as used herein, refers to the ability of an anti-CDCPI antibody or ADC to interact with CDCP1 (human or cynomolgus monkey CDCP1) with a dissociation constant (K_{D}) of about 1,000 nM or less, about 500 nM or less, about 200 nM or less, about 100 nM or less, about 75 nM or less, about 25 nM or less, about 21 nM or less, about 12 nM or less, about 11 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.3 nM or less, about 0.1 nM or less, about 0.01 nM or less, or about 0.001 nM or less. In one example, K_{D} is determined by surface plasmon resonance or Bio-Layer Interferometry, or by any other method known in the art. Bio-Layer Interferometry refers to an optical phenomenon that allows for the analysis of real-time biospecific interactions by measuring the interference patterns of reflected white light, for example using the Octet™ system (ForteBio, Pall Corp. Fremont, Calif.). For further description of the Octet™ system, see Li, B et al. (2011) Pharm. Biomed. Anal 54(2):286-294 and Abdiche, Y. N., et al. (2009) Anal. Biochem. 386(2):172-180, the contents of which are incorporated herein by reference.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.); see also Jonsson, U., et al. (1993) Ann. Biol Clin. 51:19-26; Jonsson, U., et al (1991) Biotechniques 11:620-627; Johnsson, B., et al. (1995)/Mol Recognit. 8:125-131; and Johnnson, B., et al. (1991) Anal Biochem. 198:268-277.

The term “K_{D}”, as used herein, is intended to refer to the equilibrium dissociation constant of a particular antibody-antigen interaction. The smaller the K_{D} value, the greater the binding affinity for a given target. K_{D} is calculated by kₐ/k_{d}. In one embodiment, the antibodies of the invention have a K_{D} of about about 1,000 nM or less, about 500 nM or less, about 200 nM or less, about 100 nM or less, about 75 nM or less, about 25 nM or less, about 21 nM or less, about 12 nM or less, about 11 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.3 nM or less, about 0.1 nM or less, about 0.01 nM or less, or about 0.001 nM or less.

The term “epitope” denotes a protein determinant of human CDCP1 capable of specific recognition by an antibody. Epitopes usually consist of chemically active surface groupings of molecules such as amino acids or sugar side chains and usually epitopes have specific three dimensional structural characteristics, as well as specific charge characteristics. Conformational and non-conformational epitopes are distinguished in that the binding to the former but not the latter is lost in the presence of denaturing solvents.

The term “identity” and grammatical variations thereof, mean that two or more referenced entities are the same. Thus, where two antibody sequences are identical, they have the same amino acid sequence, at least within the referenced region or portion. Where two nucleic acid sequences are identical, they have the same polynucleotide sequence, at least within the referenced region or portion. The identity can be over a defined area (region or domain) of the sequence. The % identity of a polynucleotide is determined by GAP (Needleman and Wunsch, J. Mol Biol. 48: 444-453.1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 100 nucleotides in length, and the GAP analysis aligns the two sequences over a region of at least 100 nucleotides. Most preferably, the two sequences are aligned over their entire length.

The term “pharmaceutical composition”, as used herein, means any composition, which contains at least one therapeutically or biologically active agent and is suitable for administration to the patient. Any of these formulations can be prepared by well-known and accepted methods of the art. See, for example, Gennaro, A. R., ed., Remington: The Science and Practice of Pharmacy, 20th Edition, Mack Publishing Co., Easton, Pa. (2000).

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

As used herein, the term “treating” includes alleviation of symptoms associated with a specific disorder or condition. For example, as used herein, the term “treating cancer” includes alleviating symptoms associated with cancer. In one embodiment, the term “treating cancer” refers to a reduction in cancerous tumour size. In one embodiment, the term “treating cancer” refers to an increase in progression-free survival. As used herein, the term “progression-free survival” refers to the length of time during and after the treatment of cancer that a patient lives with the disease, i.e., cancer, but does not have a recurrence or increase in symptoms of the disease. “Treatment” can also mean prolonging survival as compared to expected survival if not receiving treatment. Those in need of treatment include those already with the condition or disorder as well as those prone to have the condition or disorder or those in which the condition or disorder is to be prevented.

As used herein, the term “prevention” includes prophylaxis of the specific disorder or condition. For example, as used herein, the term “preventing cancer” refers to preventing the onset or duration of the symptoms associated with cancer. In one embodiment, the term “preventing cancer” refers to slowing or halting the progression of the cancer. In one embodiment, the term “preventing cancer” refers to slowing or preventing metastasis.

The term “therapeutically effective amount” shall be taken to mean a sufficient quantity of a CDCP1 binding protein or antibody to reduce or inhibit the growth of a CDCP1 expressing cancer to a level that is below that observed and accepted as clinically characteristic of that disorder. The skilled artisan will be aware that such an amount will vary depending on the specific antibody, fragment, and/or particular subject and/or type or severity or level of disorder Accordingly, this term is not to be construed to limit the antibody to a specific quantity.

By “subject” is meant any subject, particularly a mammalian subject, for whom diagnosis, prognosis, or therapy is desired. As used herein, the term “subject” includes any human or nonhuman animal. The term “nonhuman animal” includes all vertebrates, e.g., mammals and non-mammals, such as non-human primates, sheep, dogs, cats, horses, cows, bears, chickens, amphibians, reptiles, etc.

The term “competitively inhibits” shall be understood to mean that a protein of the disclosure reduces or prevents binding of a recited antibody (e.g. 10D7 antibody) to an epitope on CDCP1. It will be apparent from the foregoing that the protein need not completely inhibit binding of the antibody, rather it need only reduce binding by a statistically significant amount, for example, by at least about 10% or 20% or 30% or 40% or 50% or 60% or 70% or 80% or 90% or 95%. Methods for determining competitive inhibition of binding are known in the art and/or described herein. In one example, the protein and antibody are exposed to CDCP1 substantially simultaneously. Additional methods for determining competitive inhibition of binding will be apparent to the skilled artisan and/or described herein. In one example, competitive binding between two antibodies is determined using FACS analysis.

By “overlapping” in the context of two epitopes shall be taken to mean that two epitopes share a sufficient number of amino acid residues to permit an antibody that binds to one epitope to competitively inhibit the binding of an antibody that binds to the other epitope. For example, the epitopes share at least one or two or three or four or five or six or seven or eight or nine or ten amino acids.

The term “Fc” as used herein refers that that part of an antibody which is not involved directly in binding of an antibody to an antigen, but exhibits various effector functions. An Fc part of an antibody is a term familiar to those skilled in the art and is defined in the basis of papain cleavage of antibodies. Depending on the amino acid sequence of the constant region of their heavy chains, antibodies or immunoglobulins are divided in the classes: IgA, IgD, IgE, IgG and IgM, and several of these may be further divided into subclasses (isotypes), e.g. IgG1, IgG2, IgG3, and IgG4, IgA1, and IgA2. According to the heavy chain constant regions the different classes of immunoglobulins are called α, δ, ε, γ, and μ respectively.

The term “labelled anti-CDCP1 antibody or antigen binding fragment thereof” as mentioned herein refers to an antibody, or an antigen binding fragment thereof, with a label incorporated that provides for the identification of the antibody. Preferably, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides or fluorescent labels (e.g., FTTC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “antibody drug conjugate” or “ADC” as used herein refers to an anti-CDCP1 antibody or antigen binding fragment thereof which is chemically linked to one or more chemical drug(s) which may optionally be a therapeutic drug, a cytotoxic or cytostatic agent or radionuclide. In some examples the ADC is a theranostic comprising an anti-CDCP1 antibody or antigen-binding fragment thereof described herein coupled to a radionuclide which may be a diagnostic radionuclide or therapeutic radionuclide. The antibody and drug may comprise a linker that enables attachment or conjugation of the drug to the antibody. An ADC typically has anywhere from 1 to 8 drugs conjugated to the antibody, including drug loaded species of 2, 4, 6, or 8. Non-limiting examples of drugs that may be included in the ADCs are mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

The term “drug-to-antibody ratio” or “DAR” refers to the number of drugs, e.g., IGN, auristatin, or maytansinoid, attached to the antibody of the ADC. The DAR of an ADC can range from 1 to 8, although higher loads, e.g., 10, are also possible depending on the number of linkage site on an antibody. The term DAR may be used in reference to the number of drugs loaded onto an individual antibody, or, alternatively, may be used in reference to the average or mean DAR of a group of ADCs.

As used herein, reference to a CDCP1 expressing tumour is identified as having an “elevated level of CDCP1” when the level of CDCP1 is higher than in tissue surrounding the cancer. In some examples, an “elevated level of CDCP1” is one in which 5% or more of the cells in a tumour sample have membrane staining. In some embodiments a “high level” in regard to CDCP1 is 5% or more staining, for example, 5, 10, 20, 30, 40, 50, 60, 70, 80, 90, or 100% of the cells in the tumour sample are stained. In some embodiments, the protein expression levels can be measured by IHC analysis.

### CUB Domain-Containing Protein 1 (CDCP1)

CDCP1 (also known as SIMA135, TRASK, CD318 and gp140) is a glycosylated protein. The protein is a 135 kDa type I transmembrane cell surface protein. The sequence of the human protein is provided in SEQ ID NO:1 (UniProt Reference Q9H5V8). The protein is 836 amino acids in length, having a signal peptide from position 1-29 and positions 30 to 836 corresponding to the mature protein. Amino acids 30-667 correspond to the extracellular portion of the protein, amino acids 668-688 correspond to the transmembrane portion while amino acids 689 to 836 correspond to the cytoplasmic portion of the protein. Reference to CDCP1 is also intended to include a further isoform (UniProt number Q9H5V8-3) having an NK to SE substitution at 342 and 343 and amino acids 344-836 missing.

The mature protein commences at Phe30 following removal of a 29 amino acid signal peptide. The protein is localised to the cell surface. Consistent with the presence of 14 potential extracellular glycosylation sites, Western blot analysis of deglycosylated cell lysates indicates that up to 40 kDa of the difference between the apparent (˜135 kDa) and theoretical (˜90 kDa) molecular weight of mature CDCP1 is due to N-linked glycans. Western blot analysis demonstrates that CDCP1 is a phosphotyrosine protein, consistent with the presence of 5 intracellular tyrosine residues. In addition, the inhibitor PP2 has been used to demonstrate that a Src kinase family member acts to phosphorylate tyrosines of CDCP1 in HEp3 cells.

The domain structure of CDCP1 indicates that it may interact with extracellular proteins such as soluble ligands, other cell surface proteins and/or matrix components; potentially via putative CUB domains present within its amino terminal region. These structures are thought to mediate binding to a variety of protein ligands. For example, homodimerization of the MASP serine proteases acting within the lectin branch of the complement cascade is stabilized through interactions involving CUB domains (Chen C B and Wallis R (2001) J Biol Chem 276:25894-25902).

Since CDCP1 is heavily glycosylated within its extracellular domain, it is thought that ligand binding will be, at least partially, dependent on carbohydrate moieties as has been demonstrated for various isoforms of the cell surface glycoprotein CD44. Glycosylation is also thought to contribute to CDCP1 protein folding, and trafficking to and maintenance at the cell surface (Gorelik E et al. (2001) Cancer Metastasis Rev 20:245-277; Grogan M J et al. (2002) Annu Rev Biochem 71:593-634).

### Antibodies

The term “antibody” as used herein is intended to refer to full length antibodies or antigen-binding fragments thereof. Such antigen-binding fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a F(ab′)2 fragment. This fragment can be further cleaved using a thiol reducing agent, and optionally a blocking group for the Sulfhydryl groups resulting from cleavage of disulfide linkages, to produce Fab′ monovalent fragments. Alternatively, an enzymatic cleavage using pepsin produces two monovalent Fab′ fragments and an Fc fragment directly. These methods are described for example in U.S. Pat. Nos. 4,036,945 and 4,331,647; Porter, Biochem. J., 73:119 (1959); Edelman et al., Methods in Enzymology, Vol. 1, page 422 (Academic Press 1967); and Coligan et al. at sections 2.8.1-2.8.10 and 2.10.1-2.10.4.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, FV fragments include, an association of VH and VL chains. This association may be noncovalent (Inbar et al. (1972) Proc. Nat'l Acad. Sci. USA, 69:2659). Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described (Whitlow et al., Methods: A Companion to Methods in Enzymology, Vol. 2, page 97 (1991); Bird et al., Science, 242:423 (1988), Ladner et al., U.S. Pat. No. 4,946,778; and Pack et al., Bio/Technology, 11:1271 (1993)).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: A Companion to Methods in Enzymology, Vol. 2, page 106 (1991)).

In certain example, the antibody comprises a heavy chain constant region, such as an IgG1, IgG2, IgG3, IgG4, IgA, IgE, IgM, or IgD constant region. Furthermore, the antibody can comprise a light chain constant region, either a kappa light chain constant region or a lambda light chain constant region. Preferably, the antibody comprises a kappa light chain constant region

The antibodies of the disclosure may also comprise a modified Fc region to alter the effector function of the antibody. In certain examples, the mutation is S239D/A330L/1332E (3M), F243L, G236A, K322A, L234A, L235A and L234F/L235E/P331S™. In another example, the antibody comprises a modified Fc region which increases half-life. In one example, the Fc region is modified at one or more of residues 251-256, 285-290, 308-314, 385-389 and 428-436 that increases the in vivo half-life of the antibody. In certain examples, the mutation is a M252Y/S254T/T256E (YTE), or T250Q/M428L. Such modified antibodies are described in, for example EP2408813, WO 2004/029207, WO 2005/018572, WO 2014/047357, and WO 2012/130831.

Moieties that can be Coupled to the Anti-CDCP1 Antibodies

The anti-CDCP1 antibodies or antigen binding fragments described herein may be coupled to a moiety. In certain examples, the moiety selected from the group consisting of an anti-apoptotic agent, a mitotic inhibitor, an anti-tumour antibiotic, an immunomodulating agent, a nucleic acid for gene therapy, an anti-angiogenic agent, an anti-metabolite, a toxin, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, and a tyrosine kinase inhibitor.

The CDCP1 binding proteins can be fused to the moiety, e.g. the toxin, either by virtue of the moiety and binding protein being chemically synthesised, or by means of conjugation, e.g. a non-peptide covalent bond, e.g. a non-amide bond, which is used to join separately produced CDCP1 binding protein (e.g. antibody) and the moiety. Alternatively, the CDCP1 binding protein and moiety may be joined by virtue of a suitable linker peptide.

Useful toxin molecules include peptide toxins which are significantly cytotoxic when present intracellularly. Examples of toxins include cytotoxins, metabolic disrupters (inhibitors and activators) that disrupt enzymatic activity and thereby kill cancer cells, and radioactive molecules that kill all cells within a defined radius of the effector portion. A metabolic disrupter is a molecule, e.g. an enzyme or a cytokine that changes the metabolism of a cell such that its normal function is altered. Broadly, the term toxin includes any effector that causes death to a tumour cell.

Many peptide toxins have a generalised eukaryotic receptor binding domain; in these instances the toxin must be modified to prevent killing cells not bearing CDCP1 (e.g. to prevent killing cells not bearing CDCP1 but having a receptor for the unmodified toxin). Such modifications must be made in a manner that preserves the cytotoxic function of the molecule. Potentially useful toxins include, but are not limited to diphtheria toxin, cholera toxin, ricin, 0-Shiga-like toxin (SLT-I, SLT-II, SLT-IIV), LT toxin, C3 toxin, Shiga toxin pertussis toxin, tetanus toxin, Pseudomonas exotoxin, alorin, saponin, modeccin and gelanin. Other toxins include tumour necrosis actor alpha (TNF-alpha) and lymphotoxin (LT). Another toxin which has antitumor activity is calicheamicin gamma 1, a diyne-ene containing antitumour antibiotic with considerable potency against tumours (Zein N et al (1988). Science 240:1198-201).

As an example, diphtheria toxin (which sequence is known) can be conjugated to the anti-CDCP1 antibodies of the present disclosure. The natural diphtheria toxin molecule secreted by Corynebacterium diptheriae consist of several functional domains that can be characterised, starting at the amino terminal end of the molecule, as enzymatically-active fragment A (AA 1-193) and fragment B (AA 194-535) which includes a translocation domain and a generalised cell binding domain (AA 475-535).

The anti-CDCP1 antibody and the toxin moiety can be linked in any of several ways which will be known to persons skilled in the art. For example, a method of conjugating an CDCP1 binding protein to a toxin (gelonin) is described in Chu T C et al. (2006) Cancer Res 6(12)5989-5992.

The moiety can also be a modulator of the immune system that either activates or inhibits the body's immune system at the local level. For example, cytokines e.g. lymphokines such as IL-2, delivered to a tumour can cause the proliferation of cytotoxic T-lymphocytes or natural killer cells in the vicinity of the tumour.

The moiety or reporter group can also be a radioactive molecule, e.g. a radionuclides, or a so-called sensitizer, e.g. a precursor molecule that becomes radioactive under specific conditions, e.g. boron when exposed to a bean of low-energy neutrons, in the so-called “boron neutron capture therapy” (BNCT) as described in Barth et al. (1990). Scientific American Oct 1990:100-107. Compounds with such radioactive effector portions can be used both to inhibit proliferation of cancer stem cells in the tumour and to label the cancer stem cells for imaging purposes.

Radionuclides are single atom radioactive molecules that can emit either α, β, or γ particles. Suitable a particle emitting radionuclides include ²¹³Bi, ²¹²Pb, ²²³Ra, ²²⁷Th, ²²⁵Ac, ²¹At. Suitable β emitting radionuclides include ¹³¹I, ¹⁷⁷Lu ¹⁸⁶Re, ¹⁵³Sm, ⁸⁹Sr, ⁹⁰Y, ⁶⁷Cu. Radionuclide agents comprise agents that are characterized by an unstable nucleus that is capable of undergoing radioactive decay. The basis for successful radionuclide treatment depends on sufficient concentration and prolonged retention of the radionuclide by the cancer cell. Other factors to consider include the radionuclide half-life, the energy of the emitted particles, and the maximum range that the emitted particle can travel. In preferred embodiments, the therapeutic agent is a radionuclide selected from the group consisting of radionuclides that substantially decay with Auger-emitting particles.

The radioactive molecule may be tightly linked to the anti-CDCP1 antibody either directly or by a bifunctional chelate. This chelate must not allow elution and thus premature release of the radioactive molecule in vivo (Waldmann, Science, 252:1657-62 (1991)). As an example, to adapt ¹⁷⁷Lu beta therapy to the present invention, a ¹⁷⁷Lu atom can be attached to the anti-CDCP1 antibody by conjugating the metal chelator DOTA (1,4,7,10-tetraazacyclododecance tertaacetic acid) to the antibody. The ¹⁷⁷Lu will be delivered to and concentrates in or on the tumour cells by the specific binding of the anti-CDCP1 antibody to the cancer cell. The beta particles spontaneously emitted by ¹⁷⁷Lu decay are a highly lethal, but very localized, form of radiation, because particles have a path length of only about 3 mm.

In one example the radionuclide labelled anti-CDCP1 antibodies of the disclosure can be used to deliver therapeutic doses of radiation for cancer treatment. Accordingly, the present disclosure also provides for the use of radionuclide labelled anti-CDCP1 antibodies as theranostics.

Alternatively, or in addition, the anti-CDCP1 antibody is labelled with, for example, a magnetic or paramagnetic compound, such as, iron, steel, nickel, cobalt, rare earth materials, neodymium-iron-boron, ferrous-chromium-cobalt, nickel-ferrous, cobalt-platinum, or strontium ferrite.

### Antibody-Drug Conjugates (ADC)

The anti-CDCP1 antibodies described herein may be conjugated to a drug to form an anti-CDCP1 Antibody Drug Conjugate (ADC). In one example, the drug is a therapeutic agent which exerts a therapeutic effect in vivo. Antibody-drug conjugates (ADCs) may increase the therapeutic efficacy of antibodies in treating disease, e.g., cancer, due to the ability of the ADC to selectively deliver one or more drug moiety(s) to target tissues or cells, e.g., CDCP1 expressing tumours or CDCP1 expressing cells. Thus, in certain embodiments, the disclosure provides anti-CDCP1 ADCs for therapeutic use, e.g., treatment of cancer.

Anti-CDCP1 ADCs comprise an anti-CDCP1 antibody, i.e., an antibody that specifically binds to CDCP1, linked to one or more drug moieties. The specificity of the ADC is defined by the specificity of the antibody, i.e., anti-CDCP1. In one example, an anti-CDCP1 antibody is linked to one or more cytotoxic drug(s) which is delivered internally to a cancer cell expressing CDCP1.

In some examples, the ADC has the following formula (formula I):

Ab-(L-D)ₙ (I)

wherein Ab an anti-CDCP1 antibody described herein, and (L-D) is a Linker-Drug moiety. The Linker-Drug moiety is made of L- which is a Linker, and -D, which is a drug moiety having, for example, cytostatic, cytotoxic, or otherwise therapeutic activity against a target cell, e.g., a cell expressing CDCP1; and n is an integer from 1 to 20. In some embodiments, n ranges from 1 to 8, 1 to 7, 1 to 6, 1 to 5, 1 to 4, 1 to 3, 1 to 2, or is 1. The DAR of an ADC is equivalent to the “n” referred to in Formula I.

In one example, the D moieties are the same. In another example, the D moieties are different.

Drugs that may be conjugated to the anti-CDCP1 antibodies described herein include mitotic inhibitors, antitumor antibiotics, immunomodulating agents, gene therapy vectors, alkylating agents, antiangiogenic agents, antimetabolites, boron-containing agents, chemoprotective agents, hormone agents, glucocorticoids, photoactive therapeutic agents, oligonucleotides, radioactive isotopes, radiosensitizers, topoisomerase inhibitors, tyrosine kinase inhibitors, and combinations thereof. The anti-CDCP1 antibodies may also be conjugated to a nucleic acid, a peptide, a protein, a compound that increases the half-life of the molecule in a subject and mixtures thereof.

Exemplary therapeutic agents include, but are not limited to an anti-angiogenic agent, an anti-neovascularization and/or other vascularization agent, an anti-proliferative agent, a pro-apoptotic agent, a chemotherapeutic agent or a therapeutic nucleic acid. A toxin includes any agent that is detrimental to (e.g., kills) cells. Additional techniques relevant to the preparation of immunoglobulin-immunotoxin conjugates are provided in for instance in U.S. Pat. No. 5,194,594. Exemplary toxins include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, WO 93/21232.

In one example, the therapeutic agent is an ultracytotoxic agent. As used herein, the term “ultracytotoxic agent” refers to agents that exhibit highly potent chemotherapeutic properties, yet themselves are too toxic to administer alone as an anti-cancer agent. That is, an ultracytotoxic agent, although demonstrating chemotherapeutic properties, generally cannot be safely administered to a subject as the detrimental, toxic side-effects outweigh the chemotherapeutic benefit. Ultracytotoxic agents include, for example, the dolastatins (e.g., dolastatin-10, dolastatin-15), auristatins (e.g., auristatin-E, auristatin-F), maytansinoids (e.g., maytansine, mertansine/emtansine (DM1, ravtansine (DM4)), calicheamicins (e.g., calicheamicin γ1), and esperamicins (e.g., esperamicin A1), amongst others.

In one example, the anti-CDCP1 antibodies may be conjugated to at least one auristatin. Auristatins possess anticancer activity by interfering with microtubule dynamics and GTP hydrolysis, thereby inhibiting cellular division. For example, Auristatin E (U.S. Pat. No. 5,635,483) is a synthetic analogue of the marine natural product dolastatin 10, a compound that inhibits tubulin polymerization by binding to the same site on tubulin as the anticancer drug vincristine (G. R. Pettit, Prog. Chem Org. Nat. Prod, 70: 1-79 (1997)). Dolastatin 10, auristatin PE, and auristatin E are linear peptides having four amino acids, three of which are unique to the dolastatin class of compounds. Exemplary embodiments of the auristatin subclass of mitotic inhibitors include, but are not limited to, monomethyl auristatin D (MMAD or auristatin D derivative), monomethyl auristatin E (MMAE or auristatin E derivative), monomethyl auristatin F (MMAF or auristatin F derivative), auristatin F phenylenediamine (AFP), auristatin EB (AEB), auristatin EFP (AEFP), and 5-benzoylvaleric acid-AE ester (AEVB). The synthesis and structure of auristatin derivatives are described in U.S. Patent Application Publication Nos. 2003-0083263, 2005-0238649 and 2005-0009751; International Patent Publication No. WO 04/010957, International Patent Publication No. WO 02/088172, and U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein.

In one example, the anti-CDCP1 antibodies are conjugated to at least one MMAE (monomethyl auristatin E).

The chemical structure of MMAE is as follows:

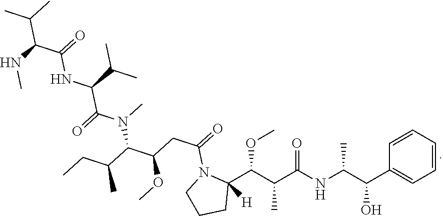 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

Monomethyl auristatin E (MMAE, vedotin) inhibits cell division by blocking the polymerization of tubulin. Because of its super toxicity, it also cannot be used as a drug itself. In recent cancer therapy developments, it is linked to a monoclonal antibody (mAb) that recognizes a specific marker expression in cancer cells and directs MMAE to the cancer cells. In one embodiment, the linker linking MMAE to the anti-CDCP1 antibody is stable in extracellular fluid (i.e., the medium or environment that is external to cells), but is cleaved by cathepsin once the ADC has bound to the specific cancer cell antigen and entered the cancer cell, thus releasing the toxic MMAE and activating the potent anti-mitotic mechanism.

In one example, the antibody is conjugated or linked to another protein for example, an immunomodulator or a half-life extending protein or a peptide or other protein that binds to serum albumin amongst others. Exemplary serum albumin binding peptides or protein are described in US2006/0228364 or US2008/0260757.

### Linkers

In some examples, the anti-CDCP1 antibody is coupled to a moiety via a linker. In some examples, the ADC comprises a linker which links the anti-CDCP1 antibody and at least one drug.

The term “linker,” as used herein, refers to a chemical moiety that may be bifunctional or multifunctional, and is used to attach the antibody and drug. In one example, the linker includes a spacer which extends the drug linkage to avoid, for example, shielding the active site of the antibody or improving the solubility of the ADC.

In one example, the linker covalently attaches an antibody to the drug.

The skilled person will appreciate that nay one of a variety of suitable linkers may be used. The linker should provide sufficient stability during systemic circulation, though allow for rapid and efficient release of the drug in an active form at its site of action, e.g. once internalised into a cancer cell.

The linker may be a cleavable linker or a non-cleavable linker. A non-cleavable linker is one which is inert or substantially inert to cleavage on exposure to in vivo conditions over the required time period. Noncleavable linkers are not cleaved under biological conditions.

Non-limiting examples of linkers include acid-labile linkers, protease sensitive linkers, photolabile linkers or disulphide-containing linkers. In one example, the linker may be cleavable under intracellular conditions. In another example, the linker is cleavable under reducing conditions. In one example, the linker is cleavable by a cleaving agent, e.g. intracellular peptidase or protease enzyme.

In some examples, the linker is not cleavable and the drug is released such as described in US 2005/0238649.

In some examples, the linker is N-succinimidyl 4-(2-pyridyldithio)-pentanoate or the highly water-soluble analog N-sulfosuccinimidyl 4-(5-nitro-2-pyridyldithio)-pentanoate, N-succinimidyl-4-(2-pyridyldithio) butyrate (SPDB), N-succinimidyl-4-(5-nitro-2-pyridyldithio) butyrate (SNPB), and N-sulfosuccinimidyl-4-(5-mtro-2-pyridyldithio) butyrate (SSNPB), N-succinimidyl-4-methyl-4-(5-nitro-2-pyridyldithio)pentanoate (SMNP), N-succinimidyl-4-(5-N,N-dimethylcOT butyrate (SCPB) or N-sulfosuccinimidyl4-(5-N,N-dimemylcarboxamido-2-pyridyld. butyrate (SSCPB)).

In one examples, the linker comprises an amino acid unit. Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline Amino acid units can be designed and optimized for enzymatic cleavage by a particular enzyme, for example, a tumor-associated protease, cathepsin B, C and D, or a plasmin protease.

In one example, the amino acid unit is valine-citrulline (vc or val-cit). In another example, the amino acid unit is phenylalanine-lysine (i.e., fk). In yet another example, the amino acid unit is N-methylvaline-citrulline. In yet another example, the amino acid unit is 5-aminovaloic acid, homo phenylalanine lysine, tetraisoquinolinecarboxylate lysine, cyclohexylalanine lysine, isonepecotic acid lysine, beta-alanine lysine, glycine serine valine glutamine and isonepecotic acid.

In one example, the linker comprises a Val-Cit-PAB group, the therapeutic agent moiety is MMAE, and the Val-Cit-PAB group is attached to the therapeutic agent moiety as follows (i.e., Val-Cit-PAB-MMAE):

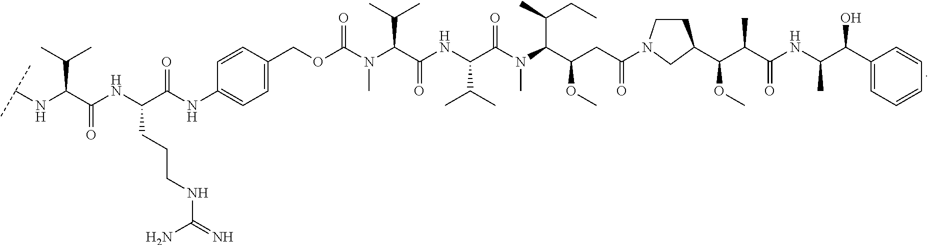

### PEGylation

The anti-CDCP1 antibodies of the present disclosure can be modified to contain additional non-proteinaceous moieties that are known in the art and readily available. For example, the moieties suitable for derivatization of the binding protein are physiologically acceptable polymer, e.g., a water soluble polymer. Such polymers are useful for increasing stability and/or reducing clearance (e.g., by the kidney) and/or for reducing immunogenicity. Non-limiting examples of water soluble polymers include, but are not limited to, polyethylene glycol (PEG), polyvinyl alcohol (PVA), or propropylene glycol (PPG).

In one example, an anti-CDCP1 antibody may comprise a plurality of PEG groups which are covalently linked to the antibody.

A PEG group is a polyethylene glycol group, i.e. a group comprising repeat units of the formula —CH₂CH₂O—. Different molecular weight PEG materials may be contemplated. In some examples, the PEG groups have an average molecular weight in the range of from 500 to 2500 Daltons. In some examples, the PEG groups have an average molecular weight in the range of from 1500 to 2500 Daltons. In some examples, the PEG groups have an average molecular weight in the range of from 1900 to 2300 Daltons. In some examples, the PEG groups have an average molecular weight in the range of from 2100 to 2500 Daltons. In some examples, the PEG groups have an average molecular weight of about 1100 Daltons. In some examples, the PEG groups have an average molecular weight of about 2000 Daltons. In some examples, the PEG groups have an average molecular weight of about 1100, about 1200, about 1300, about 1400, about 1500, about 1600, about 1700, about 1800, about 1900, about 2000, about 2100, about 2200, about 2300, about 2400 or about 2500 Daltons.

### Detection of Cancer

The anti-CDCP1 antibodies and antigen-binding fragments thereof described herein can be used in vitro for diagnostic purposes to determine the presence of cancer cells in a tissue. The method involves examining a biological sample taken from a subject wherein the sample is suspected of containing such cancer cells for the expression of CDCP1 on the cell surface. Such samples can include a tissue biopsy, blood, urine or saliva sample. For example, the biological sample can be contacted with a labelled antibody of the present disclosure (e.g. antibody 10D7) and the ability of the antibody to specifically bind to the cancer cells in the sample determined. Binding by the antibody indicates the presence of a CDCP1-expressing cancer cell. Assays which can be employed include ELISA, RIA, EIA, Western Blot analysis, immunohistological staining and the like.

In one example, immunohistochemistry can be used to diagnose cancer in an subject. For example, a sample is taken from a subject e.g. biopsy specimen from a tissue suspected of having cancer. The sample can be affixed to a slide and contacted with the anti-CDCP1 antibody. The antibody can be labelled with an enzyme, a fluorophore or radioisotope. Following binding of the antibodies to CDCP1, the position of the antibodies is determined through use of known techniques.

The anti-CDCP1 antibodies or antigen-binding proteins thereof described herein can also be used to localise CDCP1 expressing tumour cells in vivo by administering to a subject an isolated anti-CDCP1 bind antibody of the present disclosure which is labelled with a reporter group which gives a detectable signal. Bound antibodies can then be detected using flow cytometry, microscopy, external scintigraphy, emission tomography, optical imaging or radionuclear scanning. The method can be used to stage a cancer in a subject with respect to the extent of the disease and to monitor changes in response to therapy.

Detection of cancer stem cells can be facilitated by coupling the anti-CDCP1 antibody to a detectable label. Examples of detectable labels include various enzymes, prosthetic groups, fluorescent materials, luminescent materials, bioluminescent materials, electron dense labels, labels for MRI, magnetic labels and radioactive materials. Examples of suitable enzymes include horseradish peroxidise, alkaline phosphatise, p-galactosidase, or acetylcholinesterase. Examples of suitable prosthetic group complexes include streptavidin/biotin and avidin/biotin. Examples of suitable fluorescent materials include umbellifone, fluorescein isothiocyanate, rhodamine, dischlorotriazinylamine fluorescein, dansyl chloride or phycoerythrin. An example of a luminescent material includes luminol. Examples of bioluminescent materials include luciferase, luciferin, and aequorin, and examples of suitable radioactive material include ¹²⁵I, ¹³¹I, ³⁵S, ¹⁸F, ⁶⁴Cu, ^{94m}Tc, ¹²⁴I, ¹¹C, ¹³N, ¹⁵O, ⁶⁸Ga, ⁸⁹Zr, ²⁰³Pb, ⁸⁶Y, ⁸²Rb or ³H.

The radionuclide labelled CDCP1 binding proteins of the disclosure can be used to detect and stage tumours thus facilitating cancer diagnosis which can inform decisions around the design of treatment regimens.

In some examples, the anti-CDCP1 antibodies are labelled with a radiolabel via a bifunctional chelator which is a bifunctional cyclohexyl diethylenetriaminepentaacetic acid (DTPA) chelate (see U.S. Pat. Nos. 5,124,471; 5,434,287; and 5,286,850, each of which is incorporated herein by reference). In some examples, the chelator is 1,4,7,10-tetraazacyclododecance tertaacetic acid (DOTA). Other chelators will be familiar to persons skilled in the art and should also be considered part of the present disclosure.

In another example, the disclosure provides a glycosylated binding protein wherein the anti-CDCP1 antibody or antigen binding portion thereof comprises one or more carbohydrate residues

### Cancers Amenable to Treatment

The anti-CDCP1 antibodies and antigen-binding fragments thereof, labelled anti-CDCP1 antibodies and antigen-binding fragments thereof, ADCs and compositions described herein are particularly suitable for the treatment of CDCP1 expressing cancers.

The types of cancers amenable to treatment according to the methods disclosed herein include lung cancer, non-small cell lung (NSCL) cancer, bronchioalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, lymphocytic leukemia, including refractory versions of any of the above cancers, or a combination of one or more of the above cancers.

Preferably, the cancer is selected from pancreatic cancer, ovarian cancer and colorectal cancer (including combinations thereof). Preferably such cancers are characterized by CDCP1 expression or overexpression.

### Treatment and Prophylaxis

The present disclosure also provides methods of treating cancer in a subject by administering to the subject a therapeutically effective amount of an anti-CDCP1 antibody or antigen-binding fragment, labelled anti-CDCP1 antibody or antigen-binding fragment thereof, ADC or composition described herein.

The present disclosure also provides for the use of an anti-CDCP1 antibody or antigen-binding fragment, labelled anti-CDCP1 antibody or antigen-binding fragment thereof, ADC or composition described herein in the manufacture of a medicament for treating cancer in a subject.

The term “cancer” as used herein encompasses primary as well as metastatic cancers. Such cancers can be of a tissue origin of the lung, liver, kidney, mammary gland, epithelial, thyroid, leukemic, pancreatic, endometrial, ovarian, cervical, skin, colon and lymphoid tissues. In some examples, the cancer is characterised by an abnormal or overexpression of CDCP1.

The described methods and uses may also extend in some examples to prevention or prophylaxis of cancer in a subject.

The administration of the anti-CDCP1 antibody or antigen-binding protein thereof may be carried out in any convenient manner, including by aerosol inhalation, injection, ingestion, transfusion, implantation or transplantation. Preferably, they are administered to a patient by subcutaneous (s.c.), intraperitoneal (i.p.), intra-arterial (i.a.), or intravenous (i.v.) injection.

### Pharmaceutical Compositions

The anti-CDCP1 antibody or antigen-binding fragment, labelled anti-CDCP1 antibody or antigen-binding fragment thereof, ADC and compositions described herein can be formulated into pharmaceutical compositions for parenteral, topical, oral, or local administration, aerosol administration, or transdermal administration, for prophylactic or for therapeutic treatment.

The pharmaceutical compositions can be administered in a variety of unit dosage forms depending upon the method of administration. For example, unit dosage forms suitable for oral administration include powder, tablets, pills, capsules and lozenges.

The pharmaceutical compositions of this disclosure are useful for parenteral administration, such as intravenous administration or subcutaneous administration.

The compositions for administration will commonly comprise a solution of the anti-CDCP1 antibody or ADC of the disclosure dissolved in a pharmaceutically acceptable carrier, such as an aqueous carrier. A variety of aqueous carriers can be used, e.g., buffered saline and the like. The compositions may contain pharmaceutically acceptable carriers as required to approximate physiological conditions such as pH adjusting and buffering agents, toxicity adjusting agents and the like, for example, sodium acetate, sodium chloride, potassium chloride, calcium chloride, sodium lactate and the like. The concentration of the anti-CDCP1 antibody of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Non-aqueous vehicles such as mixed oils and ethyl oleate may also be used. Liposomes may also be used as carriers. The vehicles may contain minor amounts of additives that enhance isotonicity and chemical stability, e.g., buffers and preservatives.

The anti-CDCP1 antibodies of the disclosure can be formulated for parenteral administration, e.g., formulated for injection via the intravenous, intramuscular, sub-cutaneous, transdermal, or other such routes, including peristaltic administration and direct instillation into a tumour or disease site (intracavity administration). The preparation of an aqueous composition that contains the compounds of the present disclosure as an active ingredient will be known to those of skill in the art.

Suitable pharmaceutical compositions in accordance with the disclosure will generally include an amount of the anti-CDCP1 antibody of the present disclosure admixed with an acceptable pharmaceutical carrier, such as a sterile aqueous solution, to give a range of final concentrations, depending on the intended use. The techniques of preparation are generally known in the art as exemplified by Remington's Pharmaceutical Sciences, 16th Ed. Mack Publishing Company, 1980.

Upon formulation, compositions of the present disclosure will be administered in a manner compatible with the dosage formulation and in such amount as is therapeutically/prophylactically effective. Suitable dosages of compositions of the present disclosure will vary depending on the specific compound, the condition to be treated and/or the subject being treated. It is within the ability of a skilled physician to determine a suitable dosage, e.g., by commencing with a sub-optimal dosage and incrementally modifying the dosage to determine an optimal or useful dosage.

Exemplary dosages and timings of administration will be apparent to the skilled artisan based on the disclosure herein. Dosages that are therapeutically effective depend on the disease state and other clinical factors such as weight and condition of the subject, the subject's response to the therapy, the type of formulations and the route of administration. The precise dosage of a compound to be therapeutically effective can be determined by those skilled the art. As a general rule, the therapeutically effective dosage of an antibody can be in the range of about 0.5 μg to about 2 g per unit dosage form. A unit dosage form refers to physically discrete units suited as unitary dosages for mammalian treatment: each unit containing a predetermined quantity of the active material calculated to produce the desired therapeutic effect in association with any required pharmaceutical carrier. In some examples, a therapeutically effective amount of anti-CDCP1 antibody is preferably from about less than 100 mg/kg, 50 mg/kg, 10 mg/kg, 5 mg/kg, 1 mg/kg, or less than 1 mg/kg.

The methods of the present disclosure contemplate single as well as multiple administrations, given either simultaneously or over an extended period of time.

In some examples, the anti-CDCP1 antibody or antigen-binding fragment, labelled anti-CDCP1 antibody or antigen-binding fragment thereof, ADC and compositions described herein are administered in combination with one or more further pharmaceutically active agents, for example one or more further anti-cancer agents/drugs. The combination may be administered simultaneously, subsequently or separately. For example, they may be administered as part of the same composition, or by administration of separate compositions. The one or more further pharmaceutically active agents may for example be anti-cancer agents for therapy of colorectal cancer, stomach cancer, pancreas cancer, prostate cancer or breast cancer. Examples of further pharmaceutically active agents include chemotherapeutic and cytotoxic agents, small molecule cytotoxics, tyrosine kinase inhibitors, checkpoint inhibitors, EGFR inhibitors, antibody therapies, taxanes and aromatase inhibitors.

### Uses of the CDCP1 Binding Proteins in Immunotherapy

The present disclosure also encompasses the use of anti-CDCP1 antibodies described herein in chimeric antigen receptor (CAR)-T cell immunotherapy for cancer treatment. A CAR is an artificial chimeric protein in which a single chain antibody that recognises a cell surface antigen on a cancer cell is fused with a single transduction region that induces the activation of a T cell.

CARs encode for transmembrane chimeric molecules with dual function: (a) immune recognition of tumor antigens expressed on the surface of tumor cells (e.g. CDCP1 as described herein); (b) active promotion and propagation of signaling events controlling the activation of the lytic machine. CARs comprise an extracellular domain with a tumor-binding moiety, typically a single-chain variable fragment (scFv), followed by a hinge/spacer of varying length and flexibility, a transmembrane (TM) region, and one or more signaling domains associated with the T-cell signaling. The first generation CARs are equipped with the stimulatory domain of the ζ-chain; in the second generation CARs, the presence of costimulatory domains (CD28) provides additional signals to ensure full activation; in the third generation CARs an additional transducer domain (CD27, 41-BB or OX40) is added to the ζ-chain and CD28 to maximize strength, potency, and duration of the delivered signals; the fourth generation CARs include armored CARs, engineered to synthesize and deliver interleukins. Armored CARs combine the CAR functional activities with the secretion of IL-2 or IL-12 expressed as an independent gene in the same CAR vector.

A chimeric antigen receptor (CAR) recognizes cell-surface tumor-associated antigen independent of human leukocyte antigen (HLA) and employs one or more signaling molecules to activate genetically modified T cells for killing, proliferation, and/or cytokine production. Adoptive transfer of T cells expressing CAR has shown promise in multiple clinical trials.

In one embodiment, cells are removed from a patient's body (for example, blood is removed from the patient to obtain T cells) and genetically modified so that they can recognize the patient's cancer cells (for example, transfected with gene encoding a CAR) and the modified T cells reintroduced into the patient. The modified T cells, when reintroduced into the patient's body, multiply and attack cancer cells. In some embodiments, the modified T cells are cultured ex vivo prior to being reintroduced into the patient. Preferably, the T cells are not activated prior to introduction of the gene encoding the CAR.

In one example, the CAR comprises or consists of a scFv of antibody 10D7. In another example, the scFv comprises or consists of a VH having the sequence set forth in SEQ ID NO:2 and a VL having the sequence set forth in SEQ ID NO:3.

In another aspect, the present disclosure relates to a method of providing a T cell response in a subject having a CDCP1-expressing cancer, the method comprising transfecting the cells with a nucleic acid encoding a CAR as described herein and administering an effective amount of the transfected cells to the subject to provide a T-cell response. For example, the cells from the subject may be obtained from peripheral blood or umbilical cord blood. The cells may be collected by, for example, apheresis or venepuncture. The cells may be allogeneic or autologous.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

### Materials and Methods

### Generation of Antibody 10D7

Monoclonal antibody 10D7 was generated by a process called “subtractive immunisation” (S.I.) in the following manner (Brooks et al., (1993) J. Cell Biol. 122(1351-1359).

Briefly, 6-8 week old C57/BL6 female mice (16-18 g) were obtained from Charles River (Montreal, Quebec). On day 1, subconfluent cultures of HeLa cells were washed with sterile PBS and harvested by nonenzymatic cell dissociation solution (Sigma Chem Co., St Louis, Mo.). Cells were resuspended in sterile PBS (4×10⁶ cells/mL) and 2×10⁶ cells were inoculated i.p. into mice. On days 2 and 3, mice were injected i.p. with 200 mg/kg cyclophosphamide (Sigma Chem Co., St Louis, Mo.) in sterile PBS. As a tolerising (immunosuppressive) agent, cyclophosphamide suppressed the immune response towards dominant immunedeterminants present on HeLa cells. On day 15, sera were collected and analysed in the whole cell ELISA for reactivity for HeLa cells. On day 18, HeLa cells stably expressing CDCP1 (HeLa-CDCP1 cells) were harvested in the same manner as described above for HeLa cells and 2×10⁶ cells were inoculated ip. into the mice. On day 39, mice received an i.p. boost of 3×10⁶ HeLa-CDCP1 cells. On day 42, sera were collected and analyzed for reactivity toward the HeLa-CDCP1 cells and lack of reactivity towards HeLa cells using a whole cell ELISA. On day 43, the mouse with the greatest HeLa-CDCP1/HeLa ratio was sacrificed for the production of hybridomas. A control protocol was performed that was exactly the same as the S.I. protocol except that no cyclophosphamide was given.

Hybridomas were produced according to the Standard methods (Worthington et al., Br. J. Haematol. 74: 216, 1990). Briefly, the spleens and accessory lymph nodes were removed, washed and fused (4:1 spleen to myeloma) with murine myeloma cell line NSO. The cells were plated (2.5×10⁴ cells/0.1 ml HAT medium/well) in 96 well tissue culture plates (Falcon, Becton Dickinson Labware). Culture supernatants were screened by the whole cell ELISA and hybridomas from each of the immunization protocols were cloned by limiting dilution. Each positive hybridoma was cloned twice. The hybridomas producing antibodies which recognized HeLa-CDCP1 cells over HeLa cells by a factor of 2-fold or higher were selected for further characterization and testing in cancer growth and metastasis assays (Deryugina El., et al (2009) Mol Cancer Res 7(8):1197-211). Monoclonal antibodies were purified from the hybridoma conditioned media as follows. The hybridoma conditioned medium was centrifuged at 3,000 g for 30 min, concentrated 10× with an Amicon Stirred Cell Concentrator (Amicon, Beverly, Mass.) and filtered through a 0.22-um filter (Acrodisc, Gelman Sciences, Ann Arbor, Mich.). The concentrated conditioned medium was applied to a protein G Sepharose column (Pharmacia LKB Biotechnology, Piscataway, N.J.) connected to a FPLC (Pharmacia LKB Biotechnology). Monoclonal antibodies were eluted with 0.1 M glycine (pH 3.0). Purified mAbs were immediately neutralized with 1.0M Tris, pH 9.0, dialyzed against PBS and stored at −20° C.

Antibody sequencing was carried out by Monash Antibody Technologies Facility and the analysis of the heavy and light chain antibody region sequences was performed using the IMGT/V-Quest Program (The International Immunogenetics Information System; http://www.imgt.org/IMGT_vquest/vquest).

The light chain variable region CDR sequences are as follows:

(SEQ ID NO: 7; CDR1)
ENVGAY,
(SEQ ID NO: 8, CDR2)
AAS
and
(SEQ ID NO: 9, CDR3)
GQSYTYPYT.

The heavy chain variable region CDR sequences are as follows:

(SEQ ID NO: 4; CDR1)
GYSFSDFN,
(SEQ ID NO: 5; CDR2)
INPNYDST,
(SEQ ID NO: 6; CDR3)
ARLGYGYAMDY.

### Antibodies and Reagents

All chemical reagents were purchased from Sigma-Aldrich (Castle Hill, NSW, Australia) except where noted and all tissue culture reagents were from Thermo Fisher Scientific (Mulgrave, VIC, Australia) except tissue culture plastic ware which was from Corning (In vitro Technologies, Eight Mile Plains, Australia).

Antibodies 10D7 and 41-2 were described previously (Deryugina El, et al., (2009) Mol Cancer Res. 7(8):1197-211.15, Hooper J D, et al., (2003) 22(12):1783-94.46). Rabbit anti-CDCP1 (#4115), rabbit anti-p-CDCP1-Y734 (#9050), mouse anti-Src (#2110) and rabbit anti-p-Src-Y416 family (#2101) antibodies were from Cell Signaling Technology (Gold Coast, Australia). Anti-CDCP antibody 2666 was from R&D Systems (In vitro Technologies, Noble Park, VIC, Australia.

Mouse anti-GAPDH antibody was from Merck (Kilsyth, Australia). Isotype control IgG1κ was from Sigma-Aldrich (Castle Hill, Australia). Recombinant CDCP1-ECD, spanning residue 30 to 665, was described previously (Chen Y, et al., (2017) J Pharm Biomed Anal. 2017; 139:65-72.).

The anti-CDCP1 antibody 10D7 and mouse monoclonal control isotype IgG1K antibody were purified from hybridoma culture supernatant by The Walter and Eliza Hall Institute of Medical Research antibody facility (Parkville, Australia).

Anti-p-CDCP1-Y734 (#9050), -Src (#2110), -p-Src-Y416 (#2101), -PCNA (PC10), - and GAPDH (D4C6R) antibodies, and antirabbit IgG (H+L) DyLight™ 680 conjugate (#5366) and -mouse IgG (H+L) DyLight™ 800 4×PEG Conjugate (#5257) were from Cell Signaling. Rabbit anti-Cleaved-PARP antibody (ABC26) was from Merck-Millipore (Macquarie Park, Australia). Goat anti-rabbit IgG (H+L) cross-adsorbed Alexa Fluor® 594, goat anti-mouse IgG (H+L) Alexa Fluor® 647, propidium iodide (PI), Qdot 625 fluorescent probe labelling kit, wheat germ agglutinin (WGA) Alexa Fluor 488 conjugate, Alexa Fluor 488 phalloidin and DAPI solution were from Thermo Fisher Scientific and Complete EDTA-free protease inhibitor mixture was from Sigma-Aldrich. CellTiter AQueous One Solution Reagent was from Promega (Hawthorn East, Australia), and specialized blocking reagents for western blot and flow cytometry were from Rigby Laboratories (Kalbar, Qld, Australia). The maleimide activated drug linker, incorporating monomethyl-auristatin E (MMAE) for generation of antibody-drug conjugates, was maleimidocaproyl-valine-citrulline-p-aminobenzoyloxycarbonyl-MMAE (MC-VC-PAB-MMAE) and was purchased from Levena (San Diego, Calif.).

### Expression Constructs, Cell Culture, Transfections and Cell Treatments

Y734F, Y743F and Y762F mutants were introduced into a described CDCP1 expression construct (He Y, et al., (2010) 285(34):26162-73.) by site directed mutagenesis. CDCP1GFP construct was sub-cloned from a described construct (He et al., supra) into vector pEGFP-N1 (Clontech, Mountain View, Calif.). Culture media and reagents were from Thermo (Scoresby, Australia) and plasticware from Corning (Mulgrave, Australia). HEY (ATCC, Manassas, Va.) and OVMZ6 (Dong Y, et al., (2012) J Biol Chem. 287(13):9792-803.) EOC cells were cultured in RPMI and DMEM media, respectively, containing 10% (v/v) FCS (HyClone, In Vitro Technologies, Eight Mile Plains, Australia), penicillin (100 units per ml) and streptomycin (100 units per ml), at 37° C. OVMZ6 cell media contained 2 mM sodium pyruvate and 2 mM L-glutamine. Lipofectamine 2000 was used for transfections (Dong et al., supra; Wortmann A, et al., J Biol Chem. 286(49):42303-15.) to generate OVMZ6-CDCP1 cells and HEY cells stably expressing wildtype, Y734F, Y743F or Y762F CDCP1GFP. For microscopy 4-chamber 35 mm glass-bottom dishes (Cellvis, Mountain View, Calif.) were coated with poly-L-lysine (Sigma-Aldrich) then dried before cell plating. Cell treatments were: dasatinib (200 nM; Sigma-Aldrich) for 2 h before antibody as described (36); proteasome inhibitor MG132 (20 μM; Sigma-Aldrich) and lysosome acidification inhibitor chloroquine (50 μM; Sigma-Aldrich) were added 16 h before antibody.

### Flow Cytometry

Adherent cells lifted non-enzymatically were fixed (4% paraformaldehyde; 30 minutes) then incubated with 10D7 or 41-2 (5 μg/ml) in PBS/1% BSA (30 minutes; 4° C.). PBS washed cells were stained with an APC-conjugated anti-mouse secondary antibody (BioLegend, Karrinyup, Australia) in PBS/1% BSA (30 min; 4° C.). For assays assessing impact of antibodies on cell surface CDCP1, adherent HEY and OVMZ6-CDCP1 cells were untreated or treated with 10D7, 41-2 or IgG₁κ (5 μg/ml; 30 min; 37° C.) in complete medium then lifted non-enzymatically before staining (30 min, 4° C.) with PE-conjugated anti-CDCP1 antibody CD318^{-PE} (BioLegend). After washes, 20,000 events were analyzed on a BD Accuri C6 flow cytometer with data displayed as mean fluorescence intensity (MFI) calculated by subtracting the value from staining only with secondary antibody.

### Antibody Affinity

Surface plasmon resonance was performed using a Biacore T200 (GE Healthcare, Parramatta, Australia) as described (Conroy P J., et al (2014) J Biol Chem. 289(22):15384-92.) with antibodies immobilized via Protein G (Sigma) on a CM5 chip (GE Healthcare). Binding kinetics to immobilized mAbs was of serial dilutions of CDCP1-ECD (50 to 1.56 nM; 30 μl/min) with 180 s association and 600 s dissociation time at 25° C. Data were processed using BIAevaluation software with readings double-referenced by subtraction of a “buffer only” control against the reference-subtracted sensorgrams.

### Antibody Internalization

HEY and OVMZ6-CDCP1 cells were adhered overnight in 96-well black-walled, clear-bottom plates (10,000 cells/well). Using amine chemistry, antibodies were conjugated with the hydrophilic, bright pH sensor dye, pHAb (Promega, Alexandria, Australia), that fluoresces at acidic pH within endosomes and lysosomes (34). Adherent cells were incubated at 37° C. with pHAb-conjugated antibodies (5 μg/mL) with signal acquired at defined time points using a fluorescent plate reader (excitation 532 nm; emission 560 nm).

### Live-Cell Microscopy

Live-cell spinning-disk confocal imaging was performed as described (Stehbens S J, et al., (2014) 16(6):561-73.) on an environmentally controlled Nikon TI inverted microscope (Nikon, New York) equipped with a Borealis-modified Yokogawa CSU-X1 confocal head (Spectral Applied Research, Ontario Canada) and a Clara cooled interline charge-coupled device (CCD) camera (Andor Technology, Belfast, United Kingdom). Dynamics of fluorescent proteins were imaged at 37° C. using a 60×1.49 NA objective (Nikon) with imaging of CDCP1^{GFP} internalization induced by 10D7^{pH} performed at ˜1 frame/sec. Image analysis and quantification was performed using Imaris 7.1 software (Bitplane, Zurich, Switzerland) (Morton J P, et al., (2010) 139(1):292-303.). Briefly, the Spots creation wizard automatically subtracted background and detected CDCP1^{GFP} puncta which were filtered for analysis based on threshold values above 20 for ‘quality’ defined as the intensity at the centre of the puncta, Gaussian filtered by % of the spot radius (Doyon J B, et al., (2011)13(3):331-7.). Puncta were tracked over sequential frames using the Autoregressive motion particle-tracking algorithm. A maximum search distance of 0.9 am was defined to prevent false track connections to nearby spots. A gap-closing algorithm linked track segment ends to track segment starts to recover tracks that were interrupted by temporary particle disappearance. Maximum permissible gap length was set to 3 frames. Three independent assays were performed for each experiment. Quantification of track length during the period 0-5 min was determined from the 100 tracks with the highest velocity in each experimental group. The software generates images overlaying color-coded tracks onto cells, with colours corresponding to the visible spectrum (violet, tracks that moved the shortest distance; red, tracks that moved the greatest distance).

### Cell Surface Biotinylation and Western Blot Analysis

Cells were biotinylated (4° C., 1 h) with cell-impermeant EZ-link NHS-SS Biotin (1.22 mg/ml; Thermo) then PBS washed and lysed in buffer containing 20 mM HEPES, 150 mM NaCl, 1 mM EDTA, 1% Triton X-100, 1×Complete protease inhibitor cocktail (PIC) and phosphatase inhibitors (2 mM Na₃VO₄, 10 mM NaF). After centrifugation the supernatant was incubated (15 minutes, 4° C.) with ImmunoPure immobilized streptavidin beads (Thermo). After pelleting, the supernatant containing intracellular proteins was transferred to a separate tube, and the beads washed in lysis buffer containing PIC and phosphatase inhibitors. Lysates and proteins separated by cell surface biotinylation (40 μg/lane) were examined by Western blot analysis as described (He Y, et al., (2016) Oncogene. 35(4):468-78).

### PET-CT Imaging of an Intraperitoneal Clear Cell EOC PDX

Mouse experiments were approved by the University of Queensland Animal Ethics Committee. For PET/CT imaging female NOD.Cg-Prkdc^{scid} Il2rg^{tmwil}/SzJ mice (6-8 weeks; 4 per group; Jackson Laboratory, Bar Harbor, Me.) were injected intraperitoneally with cells dissociated from a previously described clear cell EOC PDX designated PH250 (Weroha S J, et al., (2014) Clin Cancer Res. 2014; 20(5):1288-97) (0.2 g/mouse of pelleted cell slurry). CDCP1 expression by PDX PH250 was assessed by immunohistochemical and Western blot analysis as described (He Y, et al., (2016) Oncogene. 35(4):468-78.). 10D7 and IgG1K control were labelled with the positron-emitting radionuclide ⁸⁹Zr as described (Zeglis B M, et al., (2015) J Vis Exp. (96): 52521)), and yield and purity determined by radio-TLC and -HPLC (Agilent, Mulgrave, Australia). Imaging commenced 3 weeks after injection of cancer cells which was sufficient time for intraperitoneal tumours to establish and before the accumulation of large, dense ascites. Imaging was performed on isoflurane anaesthetised mice injected via the lateral tail vein with 3-5 MBq of ⁸⁹Zr-DFO-10D7 or ⁸⁹Zr-DFO-IgG1κ, and was performed after 1, 24, 48, 72 and 144 h using an Inveon PET/CT (Siemens, Munich, Germany). PET image acquisition (30 minutes) was followed by CT for anatomical registration and attenuation correction, with images reconstructed and analyzed using the Inveon Research Workspace (Siemens). Ex vivo bio-distribution was assessed after the final imaging time point. Harvested tumour and organs, cleaned of blood, were weighed and radioactivity quantified using a PerkinElmer 2480 Automatic Gamma Counter (Perkin Elmer, Milton, Australia) and presented as % ID/g of tumour or tissue.

### Colony Formation Analysis

MMAE-conjugated 10D7 and IgG1K were prepared as described (Nielsen C F, et al., (2017) Oncotarget. 8(27):44605-24.53) and drug-antibody ratio (DAR) determined by reverse phase LC/MS as reported (Basa L. et al., (2013) Methods Mol Biol. 1045:285-93.) on an ABSCIEX Triple TOF spectrometer (AB SCIEX Framingham, Mass.) coupled to a Shimadzu Nexera 1D UHPLC system (Shimadzu, Rydalmere, Australia). ADC peak identification was performed as reported with DAR determined by peak area integration (Hamblett K J, et al., (2004) Clin Cancer Res. 10(20):7063-70; Yao X, et al., (2015) Breast Cancer Res Treat. 153(1):123-33). Colony-formation assays were performed as described (Crowley L C, et al., (2016) Cold Spring Harb Protoc. 2016(8):pdb.prot087171) on cells (100,000/ml) treated with the indicated concentrations of 10D7, 10D7-MMAE, IgG or IgG-MMAE overnight before being lifted and replated in 24-well plates (200 cells/well). After a defined growth period media was removed and cells gently PBS washed before staining with 0.1% crystal violet (Sigma-Aldrich) in 2% ethanol. After 20 minutes and stained cells were scanned (700 nm) on a LiCOR System (Odyssey V3.0 software) with images analyzed using the ColonyArea ImageJ plugin to quantify colony area and intensity (Guzmán C, et al., (2014) PLoS One. 9(3):e92444.).

Analysis of CDCP1 mRNA Expression in PDAC Tumours

Assessment of CDCP1 mRNA expression level in PDAC was performed by analysis of mRNA expression datasets from The Cancer Genome Atlas (TCGA) and the International Cancer Genome Consortium (ICGC) that contain CDCP1 expression data and overall survival data for 170 and 220 patients, respectively. Data for the TCGA cohort were downloaded using the UCSC Xena browser (Goldman M, Craft B, Swatloski T, Cline M, Morozova O, Diekhans M, Haussler D, Zhu J. The UCSC Cancer Genomics Browser: update 2015.Nucleic Acids Res. 2015; 43:D812-7) and data for the ICGC cohort were downloaded from the ICGC data portal (Zhang J et al Nat Biotechnol. 2019 April; 37(4):367-369). For both cohorts CDCP1 mRNA expression across the two cohorts were segregated into quartiles, and first (low expression) and fourth (high expression) quartiles were used to generate Kaplan-Meier curves with a Mantel-Cox test used to determine significance.

### Immunohistochemical Analysis of CDCP1 Expression in PDAC Tumours

Tissue-microarrays contained specimens of PDAC from 223 cases from the Australian Pancreatic Cancer Genome Initiative (APGI) that have been described previously (Waddell N et al (2015) Nature 518(7540):495-501; Chou A et al (2018) Gut 67(12):2142-2155). Patients had primary operable, untreated PDAC and underwent a pancreatectomy with tumour/normal specimens analysed by whole genome sequencing as part of the ICGC. Immunohistochemistry was performed using validated anti-CDCP1 antibody 4115 (He Y et al 2016 Oncogene (2016) 35:468-478; Harrington B S et al (2016) BJC 114:417-426).

Antigen retrieval was performed in EDTA buffer at 110° C. for 15 min in a Decloaking Chamber (Biocare Medical, MetaGene, Redcliffe, QLD, Australia), and primary antibody was applied overnight at 4° C. (1:50) with signal detected using a Novolink Polymer Detection Kit (Leica Biosystems, Mt Waverley, VIC, Australia). Staining was assessed by an anatomical pathologist (CES) blinded to clinical data. Staining was assessed using a semi quantitative scoring system of both the intensity (graded as 0, no staining; 1, weak; 2, median; or 3 strong) and percentage of positive cells (in 10% increments). The score assigned to each patient represented the average percentage of CDCP1 positive cells from two cores per patient. For generation of Kaplan-Meier survival curves, patient scores were dichotomised into those below or above the average score of the entire cohort and were assigned to “low” and “high” CDCP1 expression groups, respectively.

### Cell Lines and Patient-Derived Cells

CAPAN-1 and PANC-1 PDAC cell lines and HeLa cells were from ATCC (Manassas, Va., USA) and cultured according to the protocols of the supplier. Normal human pancreatic stellate cells (hPSC) were from ScienCell Research Laboratories (Carlsbad, Calif., USA). The PDAC patient derived cells TKCC02 TKCC05, TKCC07, TKCC09, TKCC10, TKCC15, TKCC22, TKCC23 and TKCC27 were from the APGI (Humphrey E S et al (2016) Mol Cell Proteomics. 15(8):2671-85, Al-Ejeh F et al (2014) Clin Cancer Res. 20(12):3187-97). To avoid artificial cleavage of CDCP1, cell passages were performed non-enzymatically (Versene (0.48 mM EDTA in PBS, pH 7.4)). Cells were cultured at 37° C. in a humidified 5% CO₂ atmosphere. TKCC02, TKCC10, TKCC15, TKCC22, TKCC23 and TKCC27 cells were cultured in reduced oxygen (5%). TKCC05, TKCC07, TKCC09, CAPAN-1 and PANC-1 cells were cultured in 20-21% oxygen. Using a previously described protocol, cells were stably transduced with a luciferase expression construct, one of two lentiviral CDCP1 silencing constructs (shCDCP1 #1, shCDCP1 #2) or a scramble control construct (shControl) (He Y et al (2016) Oncogene 35(4):468-478). Normal human pancreatic stellate and TKCC05 cells were stably transduced with a lentiviral vector (pLEX_307, Addgene #41392) encoding green fluorescent protein (GFP) or Monomeric Kusabira-Orange 2 (mKO2) as previously described (Bock N et al (2019) Bone Res 7:13. doi: 10.1038/s41413-019-0049-8. eCollection 2019).

### Lysate Preparation, Immunoprecipitation and Western Blot Analysis

Cells were lysed in RIPA buffer containing EDTA-free Complete protease inhibitor (1×), sodium vanadate (2 mM) and sodium fluoride (10 mM). Snap frozen tissues (2-3 mm³) were lysed using ceramic particles (Lysing Matrix D, MP Biomedicals, Seven Hills, Australia) using a FastPrep-24 Instrument (MP Biomedicals). Lysates were homogenized by passing the samples through 26-G needles and cleared by centrifugation at 14,000 g and 4° C. for 30 min. Protein concentration was quantified by micro-bicinchoninic acid assay (Thermo Fisher Scientific). For protein deglycosylation, reduced lysates were treated with PGNase F or Endo H glycosidases from NEB (Genesearch, Arundel, QLD, Australia) for 1 h at 37° C. Immunoprecipitation (IP) was performed using antibodies 10D7 and 4115 and, respectively, Protein-G and Protein-A agarose beads (Sigma-Aldrich). For antibody 10D7 IP, the lysis buffer was 1% CHAPS in PBS containing 1×EDTA-free protease inhibitors cocktail while antibody 4115 IP was performed as previously described (He Y., et al. (2010) J Biol Chem. 285(34):26162-73). Lysates (40 μg for cells and 80 μg for tissues) and immunoprecipitates were separated by SDS-PAGE under reducing conditions (except where noted), transferred onto nitrocellulose membranes, and blocked in fish gelatin blocking buffer (3% w/v in PBS). Membranes were incubated with primary antibodies diluted in blocking buffer overnight at 4° C., washed with PBS containing 0.1% Tween 20, and then incubated with appropriate secondary antibody. Signals were detected using an Odyssey Imaging System and software (LI-COR Biosciences, Millennium Science). Densitometry analyses were performed using ImageJ software.

### Determination of Number of Cell-Surface CDCP1 Molecules and Binding of 10D7 on PDAC Cells by Flow Cytometry

To quantify the number of cell surface CDCP1 receptors, flow cytometry analyses were performed using the phycoerythrin (PE) tagged anti-CDCP1 antibody CD318-PE (BioLegend, Karrinyup, Wash., Australia) and a standard curve generated using dilutions of a known concentration of PE-Quantibrite Beads (BD Biosciences, Hamilton, QLD, Australia). Cells lifted non-enzymatically were blocked in PBS/0.5% BSA (30 minutes; 4° C.) before incubation of known numbers of cells with antibody CD318-PE (0.25, 0.5 and 1 μM) with the number of receptors per cell determined according the instructions of the manufacturer. Briefly, flow cytometry analysis of cells incubated with CD318-PE, using a BD Accuri C6 cytometer, identified a saturating concentration of CD318-PE molecules per cell. The corresponding MFI value was used to interpolate the number of CDCP1 receptors per cell from a standard curve of the log₁₀ values for the number of PE molecules per QuantiBRITE PE bead against the log₁₀ of the corresponding MFI values.

For staining with 10D7, adherent cells lifted non-enzymatically were blocked in PBS/0.5% BSA (30 minutes; 4° C.) before incubation with 10D7 or isotype control IgG (5 μg/ml, 1 h at 4° C.). PBS washed cells were stained with an APC-conjugated anti-mouse secondary antibody (BioLegend) in PBS/0.5% BSA (30 min; 4° C.). After PBS washes, cells were stained with PI to assess cell death which occurred during staining and analyzed on a BD Accuri C6 flow cytometer.

### Spinning-Disk Confocal Microscopy Analysis

Labelling of antibody 10D7 with fluorescent Qdot 625 probes was performed using a kit supplied by the manufacturer. TKCC05 cells were grown on poly-Lysine treated 1 μm chamber slides (DKSH, Hallam, Australia) until 70% confluent then incubated with 10D7-Qdot. After 5, 10 and 120 min cells were fixed with 4% paraformaldehyde for 15 min at room temperature (RT), washed with PBS, then incubated with DAPI to highlight cell nuclei and Alexa Fluor 488 phalloidin to highlight cell cytoplasms (30 min at RT). Imaging was performed on a Nikon/Spectral Spinning Disc Confocal microscope.

### Antibody-Drug Conjugation

To conjugate 10D7 and IgG1K with MMAE, antibody inter-chain disulfides were first partially reduced using DTT (10 nM, 15 min, 37° C.) to generate free thiols, which were reacted with excess maleimide activated MC-VC-PAB-MMAE in 10% DSMO for 2 h at 37° C. (Nielsen C F et al., (2017) Oncotarget. 8:44605-24). Reaction impurities were removed from crude 10D7-MMAE or IgG-MMAE reaction mixtures by ultrafiltration. The drug-antibody ratio (DAR) of purified labelled antibodies was determined by reverse phase LC/MS analysis of separated light and heavy chains as reported (Basa L. et al., (2013) Methods Mol Biol. 1045:285-93). Average DAR was of 4.5 to 4.7.

### Cell Migration, Non-Adherent Growth and Survival Assays

To assess migration, PDAC cells (2.5×10⁵) were seeded in serum free media into the top chamber of 24-well Transwell chambers containing a polycarbonate nucleopore membrane (8 μm pores; Corning, Crown Scientific, Minto, NSW, Australia). Cells were then treated with 10D7 (1 or 5 μg/ml), isotype matched IgG (5 μg/ml) or PBS. The chemoattractant in the bottom well was serum containing media. After 48 h migrated cells were fixed with methanol, stained with 0.2% crystal violet and imaged by microscopy. For quantification, crystal violet was extracted with methanol and absorbance at 590 nm was measured using a POLARstar Omega plate reader (BMG Labtech, Mornington, Australia).

To measure non-adherent spheroid growth, cell suspensions (10,000/2001/well) were plated in 96-well ultra-low attachment plates (Corning) in serum free, growth factor restricted media (He Y., et al. (2016) Oncogene. 35(4):468-78) supplemented with 10D7 (5 μg/ml), isotype matched IgG (5 μg/ml) or PBS. To allow longer-term cell proliferation, 100 μl of medium was replaced with fresh medium every 3 days. Relative spheroid growth was quantified after 10 days by adding the CellTiter AQueous One Solution Reagent to wells and measuring absorbance at 490 nm using a POLARstar Omega plate reader.

For survival assays, cells (5,000/well) were plated in 96-well plates and allowed to attach for 24 h. Cells were then treated for 24 h with 10D7 (5 μg/ml) or isotype control IgG (5 μg/ml) before addition of gemcitabine (0.02 to 500 nM) and incubation for another 72 h. In assays assessing the effect of cytotoxin-conjugated antibodies, cells were treated for 12 h with 10D7-MMAE, IgG-MMAE, 10D7 or IgG (0.0625 to 1.0 μg/ml) then washed before the media was replaced with standard growth medium for 72 h. The relative number of viable cells was then measured by adding the CellTiter AQueous One Solution Reagent to each well and measuring absorbance at 490 nm as described above. In co-culture assays, mKO2 expressing TKCC05 PDAC cells (2,000 cells/well) were co-cultured with GFP expressing normal human pancreatic stellate cells (2,000 cells/well) for 24 h in a 1:1 mixture of TKCC05 and hPSC growth medium before treatments with 10D7-MMAE, IgG-MMAE, 10D7 or IgG (0.0625 to 1.0 μg/ml), IgG or 10D7 (1 μg/ml) or PBS as above. Cells were imaged by wide-field fluorescence microscopy and the total area of confluence for each cell type was quantified by image analysis using Image J software.

### In Vivo Models

Mouse experiments were approved by the University of Queensland Animal Ethics Committee. PDAC cells (2.5×10⁶) were injected subcutaneously into the flanks of NOD.Cg-Prkdcscid Il2rgtm1Wjl/SzJ (NSG) mice (6-8 weeks; Jackson Laboratory, Bar Harbor, Me.). For assays assessing the impact of antibody 10D7 on xenograft growth, two weeks after PDAC cell inoculations, mice (n=6/group) were randomized and treated i.v. every four days with PBS, 10D7 (5 mg/kg) or IgG (5 mg/kg) until the end of the assay. For assays assessing whether 10D7 improves the efficacy of gemcitabine chemotherapy, four weeks after PDAC cell inoculations, mice were randomized and treated i.v. every four days with PBS (n=12), 10D7 (n=12, 5 mg/kg) or IgG (n=12, 5 mg/kg). Half of the mice in each of the three groups also received gemcitabine i.p. treatments (125 mg/kg/week). At the end of the assay tumours were harvested, weighed and processed for assessment of histology and CDCP1 expression by immunohistochemistry or western blot analysis. For assays assessing the effect of MMAE-conjugated antibodies on xenograft growth and mouse survival, four weeks after PDAC cell inoculations, mice (8/group) were randomized and treated i.v. every two weeks with PBS, 10D7 (5 mg/kg), IgG (5 mg/kg), 10D7-MMAE (5 mg/kg) or IgG-MMAE (5 mg/kg), or every week with i.p. treatment of gemcitabine (125 mg/kg). Tumour burden was monitored by calliper measurement and tumour volume calculated as previously described (Feldman J P, Goldwasser R, Mark S, Schwartz J (2009) Journal of Applied Quantitative Methods 4(4):455-462). Tumour burden and weight results are presented as mean+/−SEM and statistical analysis was performed on the last data point using a Wilcoxon-Mann-Whitney test between groups.

Radio-Labelling of 10D7 with Zr⁸⁹

10D7 and control IgG1κ were labelled with the positron-emitting radionuclide ⁸⁹Zr as described (Zeglis B M and Lewis J S (2015) J Vis Exp 96:52521). Yield and purity of the labelled antibodies were determined by radio-TLC and -HPLC (Agilent, Mulgrave, Australia). To assess the impact of radiolabelling on 10D7 binding, the immune-reactive fraction (IRF) of 10D7-⁸⁹Zr was determined by Lindmo assay as previously described (Burvenich I J et al (2016) J Nucl Med. 57(6):974-80). Briefly, serially diluted TKCC05 cells (5×10⁶-0.156×10⁶ cells) were incubated with various amounts of 10D7-⁸⁹Zr alone or in the presence of a saturating amount of unlabelled 10D7 antibody (700 nM). After incubation (3 h at 4° C.) cells were centrifuged and the radioactivity of the cell pellet and supernatant was determined using a Wizard 2480 gamma counter (Perkin Elmer, Milton, Australia) and immunoreactive fraction (IRF) was calculated as previously described (Tolmachev V, Orlova A, Andersson K. (2014) Methods Mol Biol. 1060:309-30; Burvenich I J et al (2016) J Nucl Med. 57(6):974-80).

### PET-CT Imaging

PET/CT imaging was performed on NSG mice carrying subcutaneous PDAC cell xenografts. Two weeks after PDAC cells inoculation, mice received equivalent doses of either 10D7-⁸⁹Zr or control IgG1κ-⁸⁹Zr via the lateral tail vein (˜2.0 MBq). PET imaging was performed on isoflurane anaesthetised mice after 24, 48, 72 and 144 h using an Inveon PET/CT unit (Siemens, Munich, Germany). PET image acquisition (30 minutes) was followed by CT for anatomical registration and attenuation correction, with images reconstructed and analyzed using the Inveon Research Workspace (Siemens). Ex vivo bio-distribution was assessed after the final imaging time point. Harvested tumour and organs, cleaned of blood, were weighed and radioactivity quantified using a PerkinElmer 2480 Automatic Gamma Counter (Perkin Elmer, Milton, Australia) and presented as % ID/g of tumour or tissue.

### Statistical Analysis

In vitro assays were performed in triplicate on three independent occasions. Analyses used GraphPad Prism (GraphPad, La Jolla, Calif.) with data displayed as mean and standard error of the mean (SEM) or for non-parametric data, median and range (10-90 percentiles). Statistical significance was assessed by One-way ANOVA or Student's t-test for parametric data, and for non-parametric data the Mann-Whitney test (t-test) or Kruskal-Wallis ANOVA, with P-value <0.05 considered significant.

All statistical tests for the PDAC studies were performed using IBM SPSS Statistics 23 software (IBM Australia Ltd, St Leonards, NSW, Australia). Except where noted, the Wilcoxon-Mann-Whitney test was used in analysis comparing 2 groups while the Kruskal-Wallis test Was used for comparisons involving more than 2 groups. A value of p<0.05 was considered significant. Significance values are represented in graphs as *p<0.05, **p<0.01, ***p<0.001 and ****p<0.0001.

### Production of Chimeric 10D7 Antibody

The human/mouse 10D7 chimera was generated by cloning the VH and VL sequence into human constant region heavy and light chains as below (sequences include signalling peptides MKTFILLLWVLLLWVIFLLPGATA and MDSQAQVLILLLLWVSGTCGD (which are underlined below):

VH amino acid sequence
(SEQ ID NO: 12)
MKTFILLLWVLLLWVIFLLPGATAEVQLQQFGAELVKPGASVKISCKAS
GYSFSDFNIEWLKQSHGKSLEWIGDINPNYDSTNYNQKFKGRATLTVDK
SSSTAYMEVRSLTSEDTAVYYCARLGYGYAMDYWGQGTSVTVSSASTKG
PSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGK
VL kappa amino acid sequence
(SEQ ID NO: 13)
MDSQAQVLILLLLWVSGTCGDIVMTQSPQSMSMSVGERVTLSCKASENV
GAYVSWFQQKPDQSPKLLILAASNRYTGVPARFIGSGSATDFTLTISSV
QAEDLADYHCGQSYTYPYTFGGGTKLEIKRTVAAPSVFIFPPSDEQLKS
GTASVVCLLNNFYPREAKVQWKVDNALQSGNSQESVTEQDSKDSTYSLS
STLTLSKADYEKHKVYACEVTHQGLSSPVTKSFNRGEC

Protein expression was performed as a 1 L transient transfection in suspension-adapted Freestyle 293 cells. The chimeric 10D7 antibody was purified by affinity capture with polishing by size-exclusion chromatography and characterisation by SDS-PAGE and SEC.

### Example 1 Antibody 10D7 Causes Loss of Cell Surface CDCP1 in Ovarian Cancer Cells

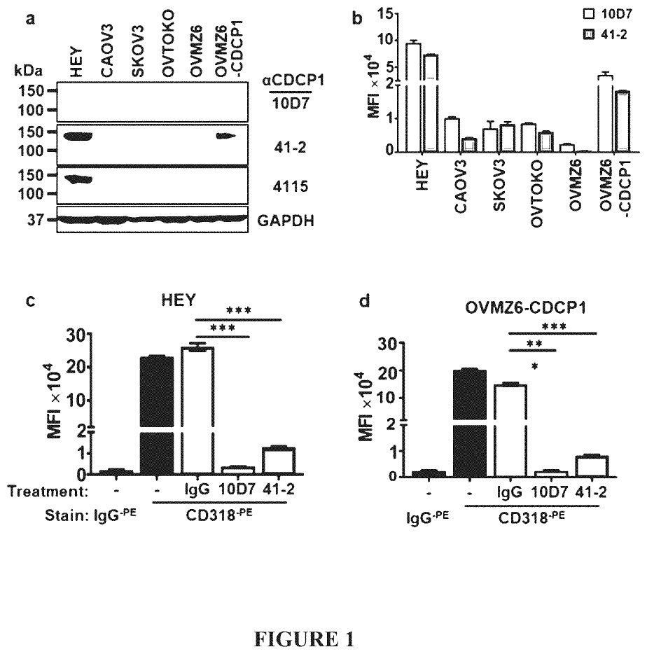

Western blot and flow cytometry analyses were employed to select epithelial ovarian cancer (EOC) cell lines to examine the mechanism of action of anti-CDCP1 mAb 10D7. Assays were performed on the five EOC cell lines HEY, CAOV3, SKOV3, OVTOKO and OVMZ6, as well as OVMZ6 cells stably over-expressing CDCP1 (designated OVMZ6-CDCP1). As shown in FIG. 1A, total CDCP1 was detected by antibody 10D7 and a comparator antibody 41-2 (another CDCP1 antibody which has been previously described in Deryugina El et al., (2009) Mol Cancer Res 7(8):1197) at similar relative levels by these antibodies, with HEY cells expressing CDCP1 at highest levels, and OVMZ6 cells the only non-expressing line. Another antibody, 4115, that recognises the intracellular carboxyl terminal of CDCP1, also detected CDCP1 at approximately the same relative level as 10D7 (FIG. 1A). Flow cytometry analysis indicated that cell surface CDCP1 levels were consistent with total levels detected by Western blot analysis. As shown in FIG. 1B, cell surface levels of CDCP1 detected with 10D7 and 41-2, were up to about 10-fold higher in HEY cells compared with CAOV3, SKOV3 and OVTOKO cells. Using antibody 10D7 OVMZ6-CDCP1 cells displayed cell surface CDCP1 levels about half those of endogenous expressing HEY cells (FIG. 1B).

Based on these analyses the highest expressing lines, HEY and OVMZ6-CDCP1, were selected to examine the impact of 10D7 on cell surface CDCP1. This was performed on adherent cells at 37° C. with 10D7, 41-2 or isotype control immunoglobulin (IgG)1κ (5 μg/ml) for 30 minutes. As shown in FIGS. 1C and D, flow cytometry analysis, performed with another anti-CDCP1 antibody tagged with the fluorophore phycoerythrin (CD318^{PE}), and generated against the CDCP1 ECD, indicated that 10D7 caused almost complete loss of cell surface CDCP1, while 41-2 reduced cell surface levels by about 90%. In contrast, IgG1κ had no effect on CDCP1.

### Example 2 Antibodies 41-2 and 10D7 Induce Internalization and Degradation of CDCP1

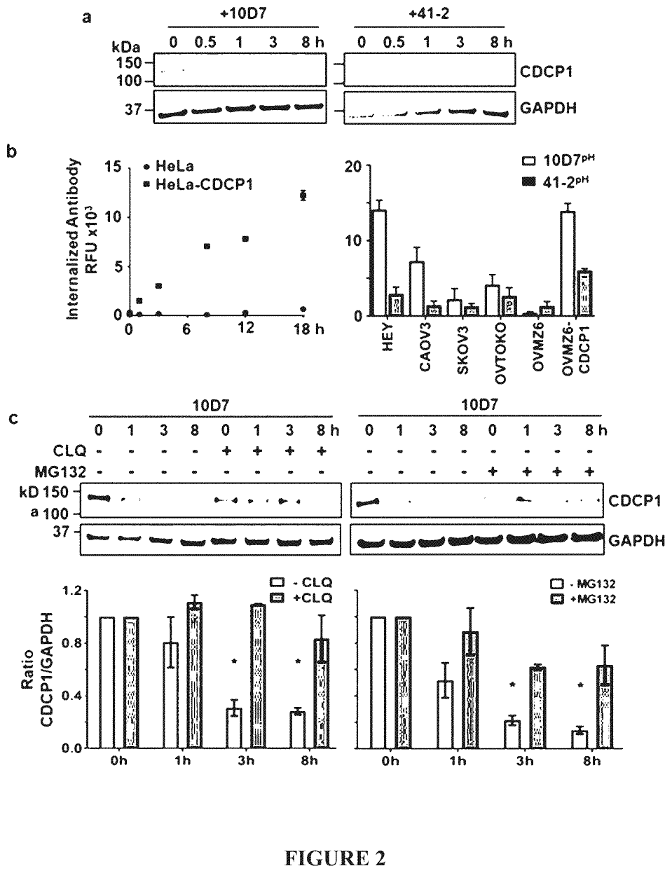

The inventors next examined the impact of 10D7 on total CDCP1 levels and whether mAb-induced loss of cell surface CDCP1 is accompanied by its degradation. This was assessed by Western blot analysis of lysates from HEY cells antibody treated for 30 min to 8 h. As shown in FIG. 2A, 10D7 caused almost complete loss of CDCP1 within 1-3 h, while 41-2 also induced loss of CDCP1 although over a slightly longer time period. To examine the mechanism of mAb-induced loss of CDCP1, the inventors employed a fluorescence-based internalization assay using mAbs labelled with a pH-sensitive dye, pHAb, which at neutral pH is not fluorescent. However, its fluorescence increases significantly as acidity increases during protein trafficking into endosomes (pH 6.0-6.5) and lysosomes (pH 4.5-5.5) (Nath N, et al., (2016) J Immunol Methods. 2016; 431:11-21).

The impact of the dye on antigen recognition was first assessed by comparing fluorescence obtained from non-CDCP1 expressing cells (HeLa) versus this line engineered to stably express CDCP1 (HeLa-CDCP1). Incubation of these cells with 10D7^{pH} resulted in increasing signal over an 18 h period from HeLa-CDCP1 but not parental HeLa cells, indicating that the pH-labelled mAb is able to recognize cell surface CDCP1 and it becomes internalized into low pH vesicles (FIG. 2B left). The assay was then performed on the six EOC cell lines using both 10D7 ^{pH} and 41-2 ^{pH}. As shown in FIG. 2B (right), after 8 h both antibodies induced robust fluorescence, indicating internalization to low pH vesicles. The signal was generally in proportion to the level of cell surface CDCP1 observed in FIG. 1B. Of note, for each of the five CDCP1 expressing lines, mAb trafficking to endosomes/lysosomes was greatest for 10D7^{pH} (FIG. 2C, right), suggesting this mAb is more effective than 41-2 at undergoing internalization.

Under basal conditions, endogenous CDCP1 is constitutively internalized then recycled to the plasma membrane or degraded via the proteasome (Adams M N, et al., (2015) Oncogene. 34(11):1375-83.8). In contrast, fluorescence-based analyses (FIG. 2B), indicated that 10D7 and 41-2 internalize to endosomes/lysosomes. Thus, the inventors were interested in the relative contribution of lysosomal and proteasomal mechanisms to mAb-induced CDCP1 degradation. HEY cells were treated with 10D7 combined with chloroquine (CLQ) to inhibit lysosomal degradation, or MG132 to inhibit proteasomal degradation. As shown in FIG. 2C, both lysosomal and proteasomal inhibition stabilized CDCP1 in response to 10D7-induced degradation, with lysosomal inhibition slightly more efficient.

### Example 3 Antibody 10D7 Binds with High Affinity within Amino Acids 30 to 358 of CDCP1

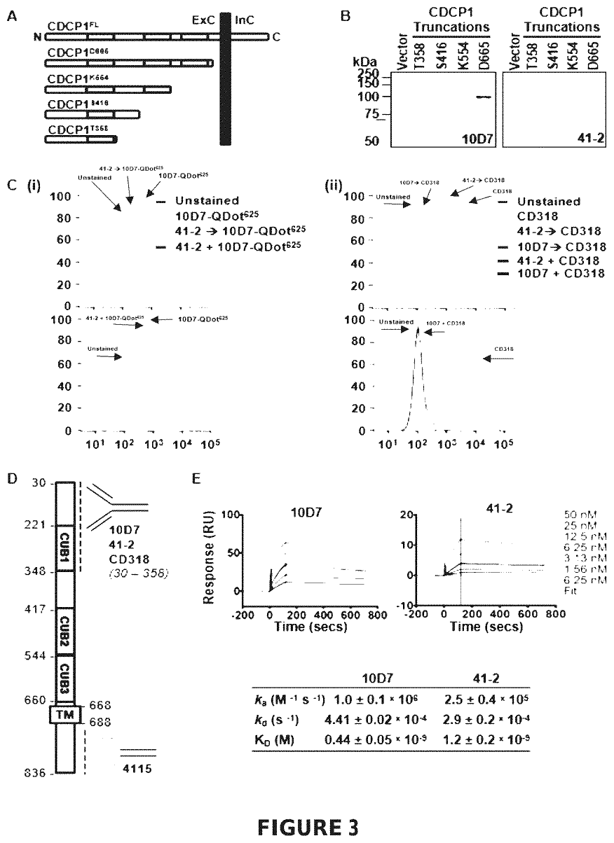

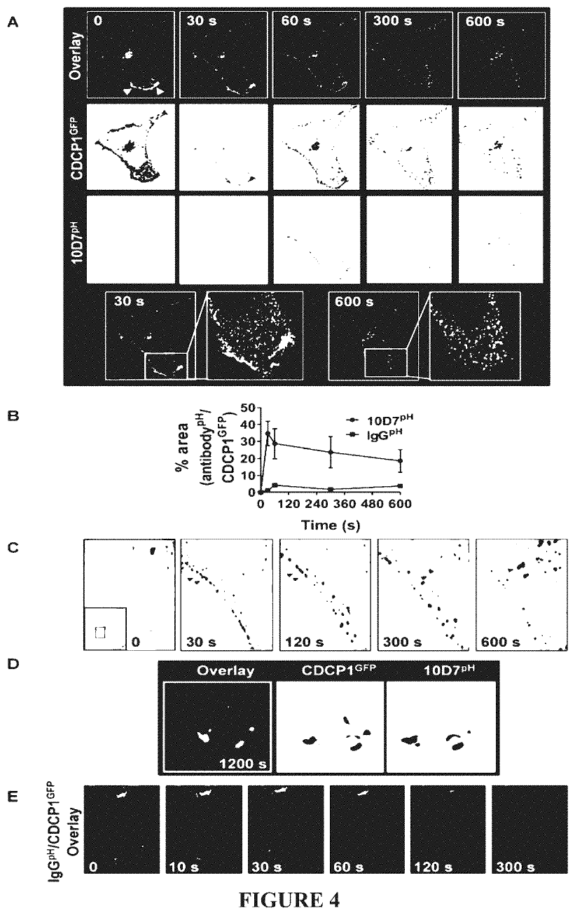

To provide additional mode of action information, the binding sites and affinities of 10D7 and comparator 41-2 were examined. Binding sites were examined by Western blot analyses of conditioned media from OVMZ6 cells transiently expressing progressively shorter carboxyl terminal truncations of CDCP1 including: the complete CDCP1 ECD (CDCP1-D665); two of the three CUB-like domains (CDCP1-K554); and one of these domains (CDCP1-S416 and -T358) (FIG. 3A). As shown in FIG. 3B, both 41-2 and 10D7 detected each of the CDCP1 truncations, with the CDCP1-S416 truncation apparent as a monomer of ˜70 kDa, and dimer of ˜140 kDa. As the CDCP1 signal peptide, spanning residues 1 to 29, is removed during cellular processing, these data indicate that both antibodies bind within amino acid 30 to 358 of CDCP1 within its ECD.

The inventors next employed two flow cytometry assay formats using high CDCP1 expressing HEY cells, to examine whether 10D7 and 41-2 compete for binding sites on CDCP1 and whether either competes with antibody CD318^{PE} for binding to CDCP1. The first assay involved incubating cells with the unlabelled competing antibody followed by fluorescently labelled 10D7 (10D7-QDot⁶²⁵) or CD318^{PE} as the detecting antibody. In the second format, cells were co-incubated with the unlabelled competing antibody and the fluorescently labelled detecting antibody. All antibodies were used at saturating concentrations in both assay formats. In assays in which cells were incubated with 41-2 followed by 10D7-QDot⁶²⁵ there was a shift in MFI to almost background levels (FIG. 3C(i) top), indicating that pre-bound 41-2 is able to largely block 10D7 binding to CDCP1. In contrast, the MFI value was largely unaffected when cells were co-incubated with 41-2 and 10D7-QDot⁶²⁵ (FIG. 3C(i) bottom), indicating that 10D7 associates more rapidly with CDCP1 than 41-2. This data suggests that while 41-2 and 10D7 compete for the same epitope and that 10D7 likely associates more rapidly with CDCP1 and has higher affinity for this receptor.

Interestingly, when cells were pre-incubated with 10D7 before incubation with CD318^{PE}, there was complete reduction in the MFI value to background levels, whereas there was only a partial reduction when cells were pre-incubated with 41-2 (FIG. 3C(ii) top). This suggests that CD318^{PE} can partially displace 41-2 but not 10D7. Also of note, co-incubation of CD318^{PE} with 10D7 or 41-2 resulted in a complete reduction in the MFI value to background levels (FIG. 3C(ii) bottom). As summarized in FIG. 3D, collectively these results indicate that 10D7 and 41-2, which bind within amino acid 30 to 358, compete with CD318^{PE} for binding sites on CDCP1. While 10D7 and 41-2 associate more rapidly than CD318^{PE} to CDCP1, CD318^{PE} can partially displace 41-2 but not 10D7, which suggests that 10D7 has higher affinity for CDCP1 than CD318.

To determine 10D7 affinity, K_{D}, surface plasmon resonance analysis was performed using the immobilized mAbs and serial dilutions of the CDCP1-ECD (50 to 1.56 nM) as the analyte. As shown in FIG. 3E, both antibody 10D7 and 41-2 had fast association (ka) and slow dissociation (kd) rates, with the K_{D} of 10D7 (0.44 nM) 2.7-fold stronger than 41-2 (1.2 nM). The stronger affinity and slightly faster association and slower dissociation rates of 10D7, compared with 41-2, explain the ability of the former to outcompete 41-2 for binding to CDCP1 that is apparent in the flow cytometry data in FIG. 3C(i) bottom, and the almost complete inability of 10D7 to displace pre-bound 41-2 noted in FIG. 3C(i) top.

### Example 4 Antibody 10D7 Induces Rapid Clustering and Lysosomal Trafficking of CDCP1

To track internalization and degradation of mAb/CDCP1 complexes, live-cell spinning-disk confocal microscopy of HEY cells stably expressing CDCP1 tagged at the carboxyl terminal with green fluorescent protein (GFP) (CDCP1 GFP) was performed. The assays were performed in HEY cells because the inventors already understood key aspects of antibody-induced degradation of endogenous CDCP1 in this line including the rate of degradation and the contribution of lysosomal and proteasomal mechanisms to these processes.

Untreated HEY-CDCP1^{GFP} cells displayed generally diffuse plasma membrane localization of CDCP1^{GFP} with prominent accentuation of signal at regions of membrane ruffling (FIG. 4A; time 0, white arrowheads, overlay and CDCP1^{GFP} panels). Within 30 seconds of 10D7 ^{pH} treatment (5 μg/ml), prominent clustering of CDCP1^{GFP} was apparent (FIG. 4A, 30 s; and inset). Concomitantly, there was almost complete loss of CDCP1 ^{GFP} at membrane ruffles. In these images coincident signal of CDCP1 ^{GFP} and 10D7^{pH} is apparent as white puncta, indicating formation of 10D7/CDCP1 complexes. After 300 seconds, 10D7^{pH} fluorescence was further increased indicating its increasing internalization and accumulation in acidified intracellular vesicles (FIG. 4A, 300 s, 10D7 pH panels). Over time, the size of white puncta increased indicating increased accumulation of CDCP1^{GFP}/10D7 ^{pH} complexes. After 300 secs, 10D7^{PH} fluorescence was further increased indicating its increasing internalisation and accumulation in acidified intracellular vesicles (FIG. 4A, 300 s 10D7^{PH} panels). Over time the size of white puncta increased indicating internalisation and accumulation within low pH intracellular vesicles (FIG. 4A, 300 s, 10D7^{pH} panel). Quantitative analysis indicated that formation of CDCP1^{GFP}/10D7^{pH} complexes occurred rapidly with maximum overlap after approximately 30 seconds and this was sustained to 600 seconds (FIG. 4B). High-resolution microscopy analysis further highlighted the 10D7-induced clustering of CDCP1^{GFP} occurring rapidly at the plasma membrane is followed by trafficking of 10D7/CDCP1 complexes into the cytoplasm (Figure. 4C, arrows). It also delineated trafficking of 10D7/CDCP1 complexes to intracellular vesicles, with signal from CDCP1^{GFP} (green) and 10D7 ^{pH} (purple) largely coincident and localised to vesicle membranes (FIG. 4D). In contrast with this rapid 10D7-induced trafficking, IgG^{pH} had no impact on CDCP1 location (FIG. 4E).

### Example 5 10D7-Induced Internalization and Degradation of CDCP1 is Mediated by Src

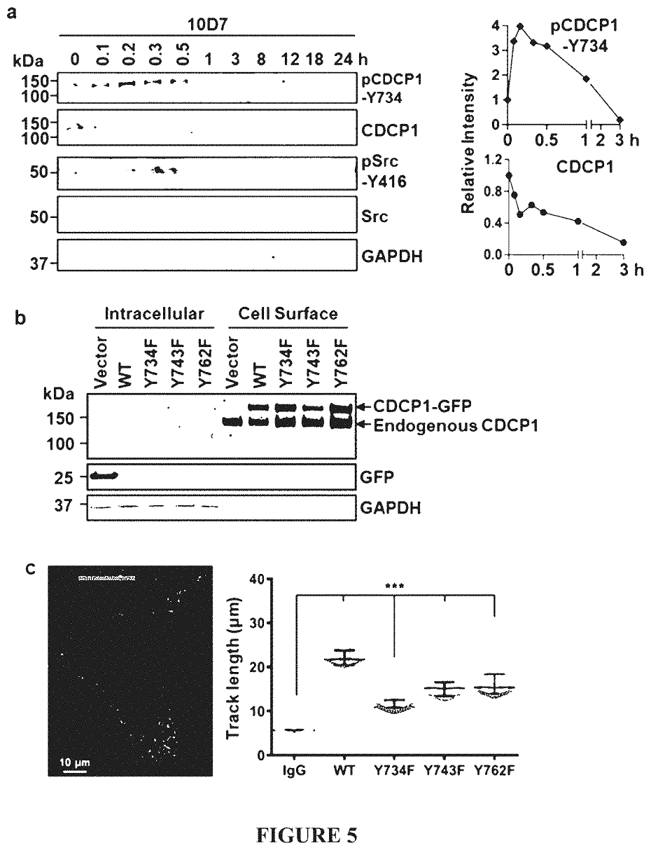

Since Src tyrosine phosphorylation is important in CDCP1-mediated signal transduction (Casar B, et al., (2012) Oncogene. 31(35):3924-38), the inventors next examined this in antibody induced processing of CDCP1. Western blot analysis of lysates from HEY cells treated for up to 24 h with 10D7, indicated that 10D7 causes rapid phosphorylation of CDCP1-Y734 within 5 min with levels then gradually reducing as CDCP1 was degraded (FIG. 5A). Interestingly, 10D7-induced phosphorylation of CDCP1 was accompanied by rapid transient activation of Src, indicated by auto-phosphorylation at Y416, over the same time course as p-CDCP1-Y734 (FIG. 5A). To examine the role of the three known Src phosphorylation sites of CDCP1 in 10D7-induced cellular processing, the inventors employed live-cell confocal microscopy analysis of HEY cells stably expressing CDCP1^{GFP} or the Y734F, Y743F and Y762F mutants. Western blot analysis of cell fractions collected from these lines by cell surface biotinylation, confirmed that wild-type CDCP1 ^{GFP} and the mutants were expressed on the plasma membrane of HEY cells together with endogenously expressed CDCP1 (FIG. 5B). Live-cell images of the four cell lines were analysed to determine the distance moved (track length) by individual CDCP1^{GFP} positive puncta during mAb-induced internalization (FIG. 5C, left). This showed that 10D7-induced internalization was significantly reduced in cells expressing Src phosphorylation site mutant CDCP1 compared to the wild-type protein (FIG. 5C, right; median track length: wild-type (WT) 21.7 μm, Y734F 10.8 μm, Y743F 15.2 μm, Y762F 15.4 μm). Of note, none of the mutations reduced track length to the background induced by the control IgG. These data indicate that Y734, Y743 and Y762 mediate, but are not essential for 10D7-induced internalization of CDCP1.

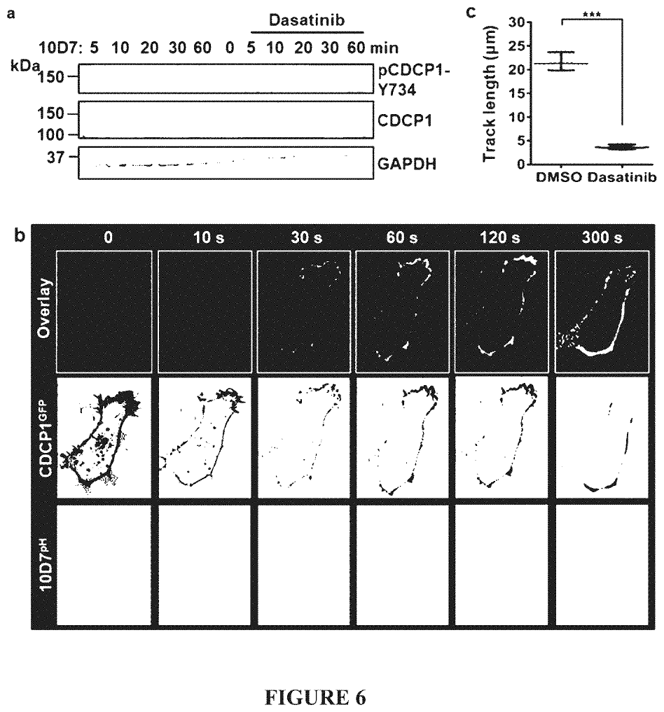

To directly examine the role of tyrosine-phosphorylation in 10D7-induced CDCP1 internalization, the inventors used the Src kinase inhibitor dasatinib. Pre-treatment with dasatinib completely blocked the generation of endogenous p-CDCP1-Y734 that is rapidly induced in response to 10D7 within 1 hour of treatment (FIG. 6A). The effect on 10D7-induced CDCP1 internalization, examined by live-cell imaging, showed that in response to dasatinib both CDCP1^{GFP} and 10D7 ^{pH} remained largely at the plasma membrane (FIG. 6B). Interestingly, although antibody and receptor were not internalized, overlapping signal from CDCP1 ^{GFP} and 10D7 ^{pH} in low pH cellular structures was apparent as dense white puncta located on the plasma membrane (FIG. 6B). Consistent with the marked reduction in 10D7-induced internalization of CDCP1^{GFP}, quantitative analysis indicated that dasatinib reduced CDCP1^{GFP} track length by about 80% (FIG. 6C). Collectively, these data demonstrate that 10D7-induced internalization of CDCP1 requires the action of a kinase, most likely Src, and that while phosphorylation of CDCP1 is evident, it is important but not essential for efficient 10D7-induced internalization of CDCP1.

### Example 6 In Vivo Accumulation of Antibody 10D7 in EOC

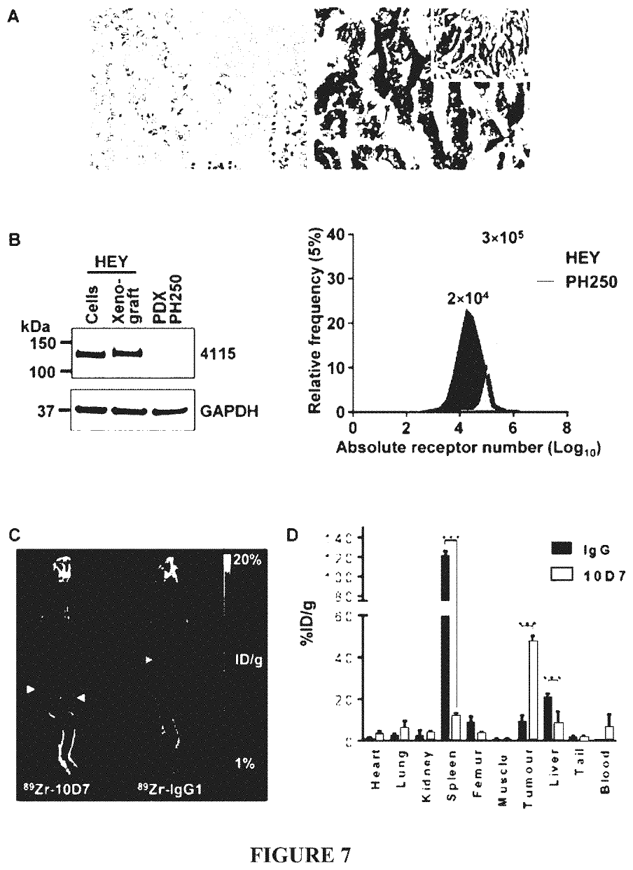

To investigate the potential for 10D7 to target CDCP1 expressing cells in EOC in vivo, a positron emission tomography (PET)-computed tomography (CT) imaging study was conducted. This was performed on mice bearing a previously described intraperitoneal patient derived xenograft (PDX) designated PH250 that has confirmed clear cell EOC pathology (FIG. 7A, left) (Weroha S J, et al., (2014) Clin Cancer Res. 20(5):1288-97.).

Immunohistochemical analysis indicated that CDCP1 is strongly expressed in this PDX predominantly on the cell surface (FIG. 7A, middle), as the full-length 135 kDa form that contains the region recognized by 10D7 (FIG. 7A, right).

Western Blot analysis indicated that CDCP1 is expressed as the full-length 135 kDa form, which contains the region recognised by 10D7, and that its expression is lower in patient-derived xenograft PH250 (PDX-PH250) compared with HEY cells grown in vitro or as xenografts in mice (FIG. 7B). Consistently, flow cytometry analysis established that cell surface CDCP1 receptor numbers are approximately 15 times higher on HEY cells (approx. 300,000/cell) than cells isolated from PH250 xenografts (approx. 20,000/cell). This suggests that PH250 xenografts of HEY cells were an appropriate model to assess the sensitivity of a CDCP1-targeting to detect EOC in vivo.

PET imaging was performed 3 weeks post inoculation of PDX PH250 with 10D7 and IgG1κ labelled with the positron-emitting radionuclide ⁸⁹Zr, achieving chemical yields of 81% and 78% respectively, with purity of >95%. Specific accumulation of ⁸⁹Zr-DFO-10D7 but not ⁸⁹Zr-DFO-IgG1κ in subcutaneous tumours was observed (FIG. 7C). Ex vivo bio-distribution analysis demonstrated percent injected dose per gram of tissue (% ID/g) values significantly higher in tumour for ⁸⁹Zr-DFO-10D7 (47.7±2.6% ID/g) compared with ⁸⁹Zr-DFO-IgG1κ (9.7±2.5% ID/g; P=0.017) (FIG. 7D). Of note and consistent with the images in FIG. 7C, ⁸⁹Zr-DFO-IgG1K showed significant accumulation in spleen (122.1±3.9% ID/g) and liver (21.2±1.4% ID/g) (FIG. 7D). This contrasted with signals from five other normal organs and the site of injection (tail) and blood, which were the same for each antibody (FIG. 7D). The variability of the ex vivo biodistribution of 10D7 signal in tumours was due to difficulty in accurately weighing, post mortem, the large number of relatively small solitary tumour deposits which were generally <3 mm in diameter. These data indicate that 10D7-based agents effectively accumulate in CDCP1-expressing EOC tumours in vivo.

### Example 7 Cytotoxin-Conjugated 10D7 Inhibits Proliferation of CDCP1 Expressing EOC Cells In Vitro

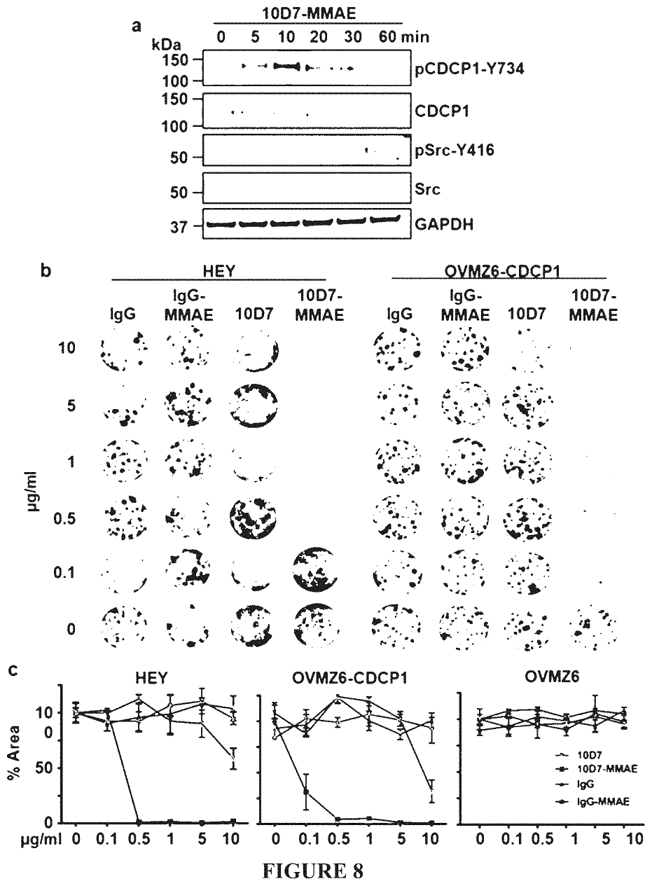

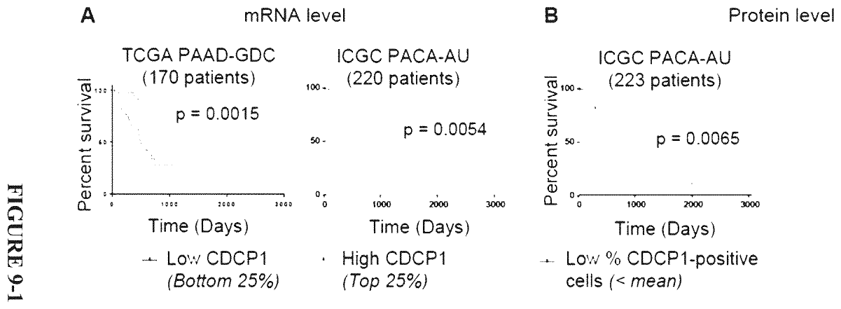

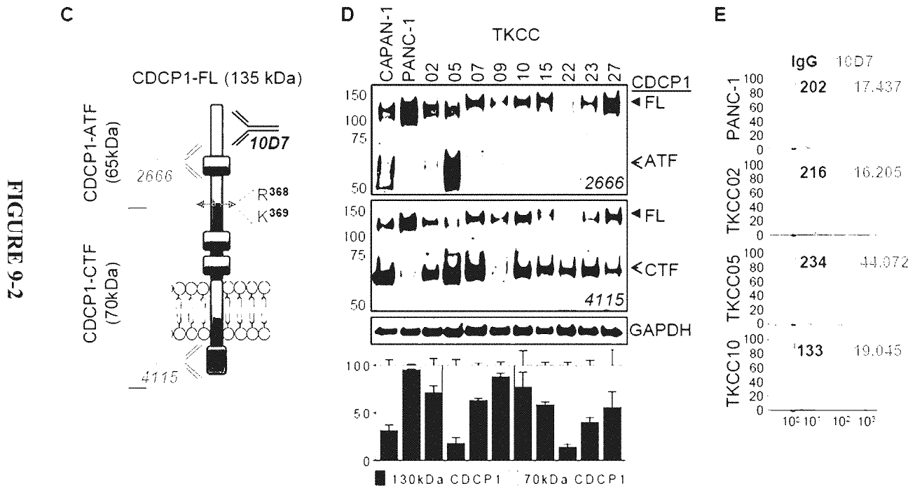

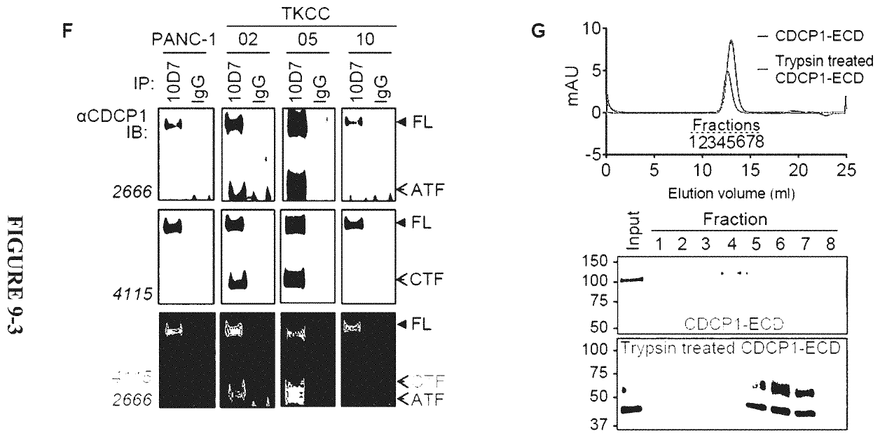

The ability of 10D7 to target a cytotoxin for lysosomal release to inhibit colony formation of CDCP1 expressing EOC cells was next examined. 10D7 was conjugated with the highly potent cytotoxin monomethyl auristatin E (MMAE) via a link incorporating a lysosomal protease cleavage site (Kamath A V, et al. (2015) Pharm Res. 32(11):3470-9.). The average drug-antibody ratio DAR) achieved for 10D7-MMAE from six preparations of 10D7-MMAE was 4.5 to 4.7). The generated antibody-drug conjugate (ADC) 10D7-MMAE was functionally active retaining the ability of the “naked” mAb to induce phosphorylation of CDCP1-Y734 and Src-Y416 within 1 hour of treatment (FIG. 8A).

To evaluate potency and selectivity, the anti-proliferative effects of 10D7-MMAE on HEY, OVMZ6 and OVMZ6-CDCP1 cells were compared with IgG-MMAE, 10D7 and IgG. While the control antibodies IgG, IgG-MMAE and 10D7 had little impact, colony formation of CDCP1 expressing HEY and OVMZ6-CDCP1 cells was sensitive to low concentrations of 10D7-MMAE (FIG. 8B). Of note, OVMZ6 cells were unaffected by 10D7-MMAE even at the highest concentration, whereas the half maximum dose required to block colony formation of HEY and OVMZ6-CDCP1 cells was approximately 0.2 μg/ml and 0.07 μg/ml, respectively (FIG. 8C). These data indicate that 10D7 can specifically deliver potent cytotoxic drugs to EOC cells via cell surface CDCP1 to inhibit proliferation.

### Example 8 Elevated Expression of CDCP1 is Associated with Poor Pancreatic-Ductal Adenocarcinoma (PDAC) Patient Outcome

To determine the prognostic value of CDCP1 in PDAC and the proportion of patients who could potentially benefit from a CDCP1 targeted therapy, its mRNA and protein expression was examined in independent patient cohorts. To determine whether elevated CDCP1 mRNA expression correlates with poorer overall survival, independent transcriptomic datasets from the TCGA (PAAD-GDC) and ICGC (PACA-AU) were analysed. Segregation of expression levels into quartiles demonstrated that in both TCGA and ICGC cohorts, patients in the top quartile of CDCP1 expression had significantly shorter survival (p=0.0015 and p=0.0054 respectively) compared to those in the bottom quartile (FIG. 9A).

CDCP1 protein expression was next examined by employing immuno-histochemical analysis of two further independent PDAC cohorts from the Australian Pancreatic Cancer Genome Initiative (APGI). These were a cohort of 223 APGI patients that had primary operable, untreated PDAC and underwent a pancreatectomy with tumour/normal specimens analysed by whole genome sequencing as part of the ICGC (Waddell N et al (2015) Nature 518(7540):495-501; Chou A et al (2018) Gut 67(12):2142-2155). Staining for CDCP1 was performed with antibody 4115 which detects the intracellular carboxyl-terminal of CDCP1-FL and CDCP1-CTF but cannot distinguish between the intact and cleaved receptor. CDCP1 expression was detected in 97% of PDAC cases, but not observed in normal pancreas, brain, salivary gland, spleen, liver or muscle, which were arrayed as controls. Kaplan-Meier survival analysis demonstrated that patients with high CDCP1 expression had significantly shorter overall survival compared to patients with low expression (p=0.0065 ICGC cohort) (FIG. 9B). In summary, mRNA and protein analyses demonstrate that CDCP1 is elevated in the vast majority of PDAC tumours and it is not expressed by the normal pancreas. CDCP1 expression and patient survival are inversely correlated, which is suggestive that CDCP1 is functionally involved in progression of PDAC.

### Example 9 Differential Proteolytic Processing of CDCP1 in PDAC Cells

Western blot analysis was performed to identify cells that are suitable to study the role of CDCP1 in PDAC examining lysates from nine previously described patient-derived PDAC cells (Chou A et al (2018) Gut 67(12):2142-2155) and two well described PDAC cell lines, CAPAN-1 and PANC-1 (Deer et al (2010) Pancreas. 39(4):425-35). Two antibodies were employed (antibody 2666 and antibody 4115) that detect the three forms of CDCP1 (135 kDa CDCP1-FL, 65 kDa CDCP1-ATF and 70 kDa CDCP1-CTF, FIG. 9C). Rabbit antibody 4115 recognises the intracellular carboxyl-terminal of CDCP1 and detects CDCP1-FL and CDCP1-CTF, while mouse monoclonal antibody 2666 detects CDCP1-FL and CDCP1-ATF (FIG. 1C). These analyses revealed that CDCP1 is expressed and cleaved to varying levels by each of the 11 PDAC cells (FIG. 1D). Analysis with antibody 4115 indicated that CDCP1 is robustly cleaved in nine PDAC lines (CAPAN-1, TKCC02, TKCC05, TKCC07, TKCC10, TKCC15, TKCC22, TKCC23, TKCC27) with much lower levels of cleavage in the remaining two lines (PANC-1, TKCC09). Surprisingly, analysis with antibody 2666 indicated that 65 kDa CDCP1-ATF, which was previously identified as being shed from the cell surface (He Y et al (2010) J Biol Chem 285(34):26162-73; He Y et al (2017) Biol Chem 399 (9):1091-1097Chen Y et al (2017) J Pharm Biomed Anal. 139:65-72), was detectable at high levels in lysates from CAPAN-1 and TK0005, much lower levels in TK0002, TK0007, TKCC15, TKCC22, TKCC23 and TKCC27, and with longer exposure times it was also present at low levels in lysates from TKCC10 cells (FIG. 1D).

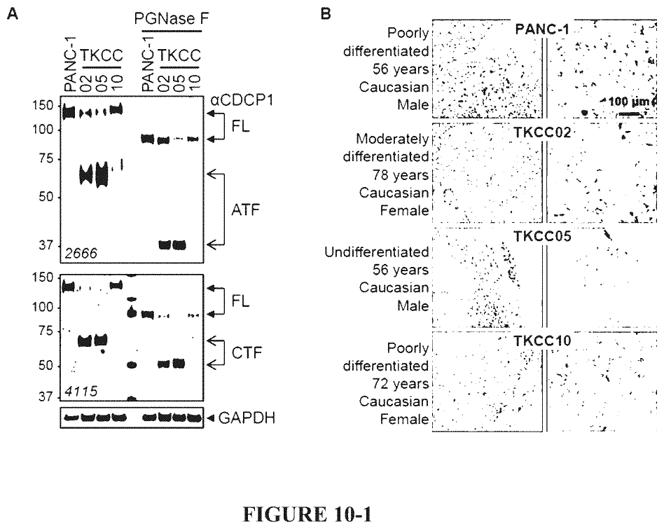

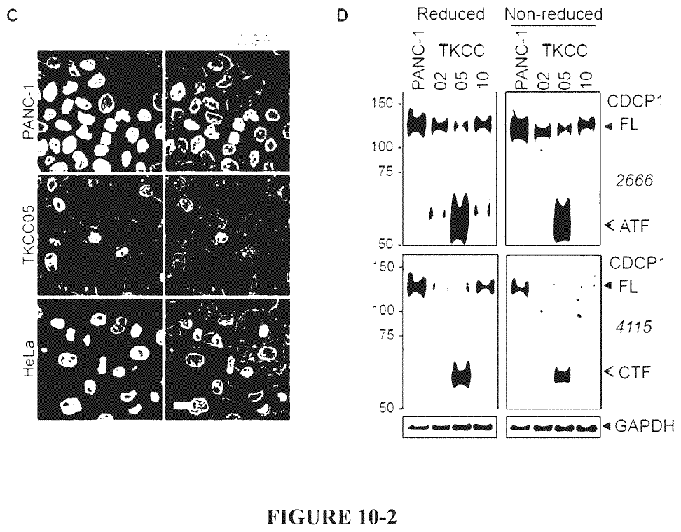

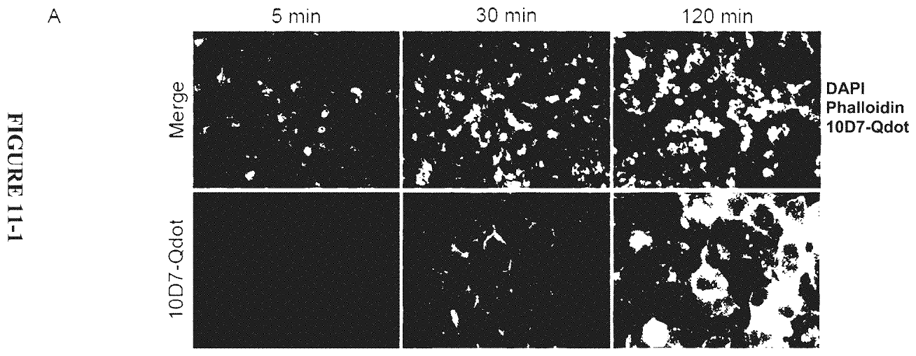

The CDCP1-ATF signal is apparent as a broad smear centred at ˜65 kDa that consists of more than one band in CAPAN-1 and TK0005 cells (FIG. 1D, 2666 western blot panel). Western blot analysis of lysates from PNAC-1, TK0002, TK0005 and TKCC10 cells treated with the amidase PNGase-F, demonstrated that this is due to N-glycosylation because the broad CDCP1-ATF signal reduced by about 25 kDa to a defined band of about 40 kDa which is close to the predicted molecular weight of this fragment of 37.9 kDa (FIG. 10A). Similarly, the amount of N-linked glycans on CDCP1-CTF was about 15 kDa with deglycosylation reducing the molecular weight from about 70 kDa to about 55 kDa which is also close to the predicted molecular weight of this fragment of 52.2 kDa (FIG. 10A). As previously reported, CDCP1-FL contained about 40 kDa of N-linked glycans reducing from 135 kDa to about 95 kDa (FIG. 10A) which is close to the predicted molecular weight of 90.1 kDa of the amino acid sequence of CDCP1 without its 29 residue signal peptide (Hooper J D., et al Zijlstra A, (2003) Oncogene. 22(12):1783-94. These data indicate for the first time that CDCP1-ATF can be retained by PDAC cells after proteolytic cleavage, which contrasts with previous reports showing that is shed from the cell surface after cleavage of CDCP1.

To investigate the mechanism by which CDCP1-ATF remains cell associated, the inventors performed further analyses on PANC-1, TKCC02, TKCC05 and TKCC10 cells. These lines display different levels of cleavage of CDCP1 (FIG. 9D), and subcutaneous mouse xenografts exhibit histological features that are representative of the landscape of PDAC pathology from undifferentiated (TKCC05, about 10% CDCP1-FL) to poorly differentiated (PANC-1, about 95% CDCP1-FL; TKCC10, about 50% CDCP1-FL) and moderately differentiated (TKCC02, about 30% CDCP1-FL) tumours (FIG. 10B). Flow cytometry analysis with anti-CDCP1 antibody 10D7, suggested that CDCP1-ATF remains cell associated via tethering to the cell surface (FIG. 9E). CDCP1 signal was approximately proportional to the total level of expression of CDCP1 rather than to the level of intact CDCP1-FL. TKCC05 cells displayed CDCP1 signal (MFI=44,072; FIG. 9E) which was more than twice the signal observed in PANC-1, TKCC02 and TKCC10 cells (MFI=17,437; 16,205 and 19,045 respectively; FIG. 9E), despite the former displaying the lowest level of intact CDCP1-FL compared to the other three cells (FIG. 9C).

Immunofluorescent confocal microscopy analysis with this antibody demonstrated consistent data. TKCC05 cells stained much more strongly for cell surface CDCP1 than PANC-1 cells despite the former expressing much lower levels of intact CDCP1-FL, with HeLa cells, which do not express CDCP1, serving as a negative control, displaying no signal (FIG. 10C).

To examine whether CDCP1-ATF remains tethered to the cell surface via binding to CDCP1-FL or CDCP1-CTF, the inventors next performed immunoprecipitation (IP) assays with antibody 10D7 detecting interacting proteins by western blot analysis with anti-CDCP1 antibodies 2666 and 4115. Consistent with CDCP1-ATF remaining tethered to the cell surface via CDCP1, antibody 4115 detected not only uncleaved CDCP1-FL but also cleaved CDCP1-CTF at high levels in both TKCC05 and to a lesser extent TKCC02 cells (FIG. 9F). CDCP1-ATF could only have been detected by this antibody if it remains linked to CDCP1-FL or CDCP1-CTF after proteolysis (FIG. 9F). No CDCP1-ATF signal was detected from PANC-1 or TKCC10 likely because these cells display lower levels of cleavage of CDCP1. Similarly, antibody 2666 detected not only CDCP1-FL but also CDCP1-CTF from TKCC05 and to a lesser extent TKCC02 cells, which could only have occurred if CDCP1-ATF remains linked to CDCP1-FL or CDCP1-CTF after proteolysis (FIG. 9F). Note that lower molecular weight signal in PANC-1, TKCC02 and TKCC10 lanes was due to reaction of the anti-mouse secondary antibody with mouse monoclonal antibody 10D7 used for IP (FIG. 9F).

The existence of stable interactions between CDCP1-ATF and CDCP1-CTF was confirmed by analysis of fractions from size-exclusion chromatographic separation of the products of trypsin cleavage of the recombinant CDCP1 extracellular domain (ECD), compared with uncleaved CDCP1-ECD. UV-Vis spectroscopy and reducing gel analysis of fractions demonstrated that the CDCP1-ECD cleavage products of ˜45 and 55 kDa failed to separate and co-eluted in fractions 5 to 7 with intact ˜110 kDa CDCP1-ECD (FIG. 9G).

To assess whether linkage of CDCP1-ATF with CDCP1-FL or CDCP1-CTF is via a disulphide bond the inventors performed anti-CDCP1 western blot analysis with antibodies 4115 and 2666 of lysates separated under both reducing and non-reducing conditions. Analysis of PANC-1, TKCC02, TKCC05 and TKCC10 cells revealed the same protein bands observed from assays under both conditions indicating that CDCP1-ATF is not linked to CDCP1-FL or CDCP1-CTF via a disulphide bond (FIG. 10D).

In summary, these data demonstrate that CDCP1 is differentially cleaved and N-glycosylated in PDAC cells producing CDCP1-ATF which remains tethered to the cell surface via non-disulfide bond interactions with CDCP1-FL or CDCP1-CTF.

### Example 10 Function Blocking Antibody 10D7 Induces Rapid Phosphorylation, Internalization and Degradation of Differentially Cleaved CDCP1 in PDAC Cells

Antibody 10D7 effectively blocks CDCP1 function inhibiting its roles in mouse models of vascular metastasis of prostate cancer (Deryugnia El et al., (2007) Mol Can Res 7:1197-1211) and intraperitoneal progression of ovarian cancer (He Y., et al (2016) Oncogene. 35(4):468-78; Harrington B S., et al. (2016) British Journal of Cancer. 114(4):417-26). At a molecular level 10D7 induces rapid Src-mediated tyrosine phosphorylation of CDCP1-Y734 followed sequentially by its clustering on the surface of ovarian cancer cells then internalisation and degradation of the receptor/antibody complex in vitro and mouse models (see Example 4).

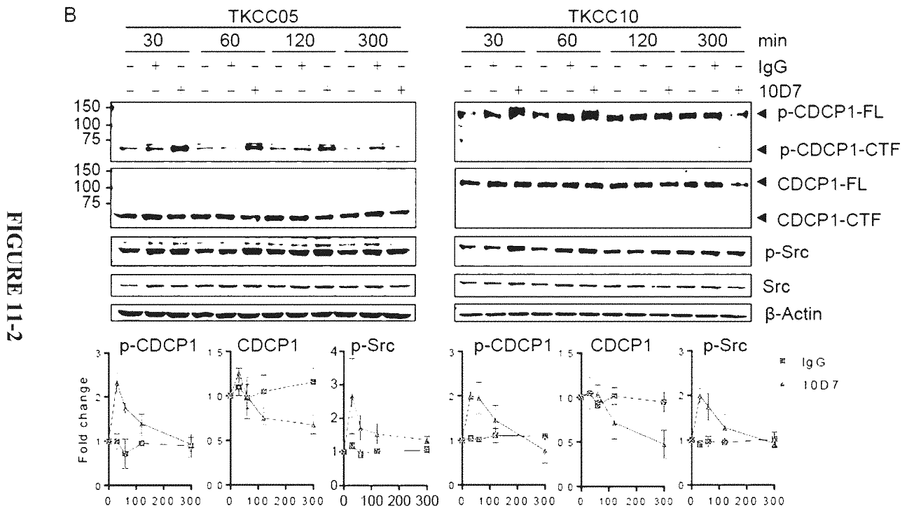

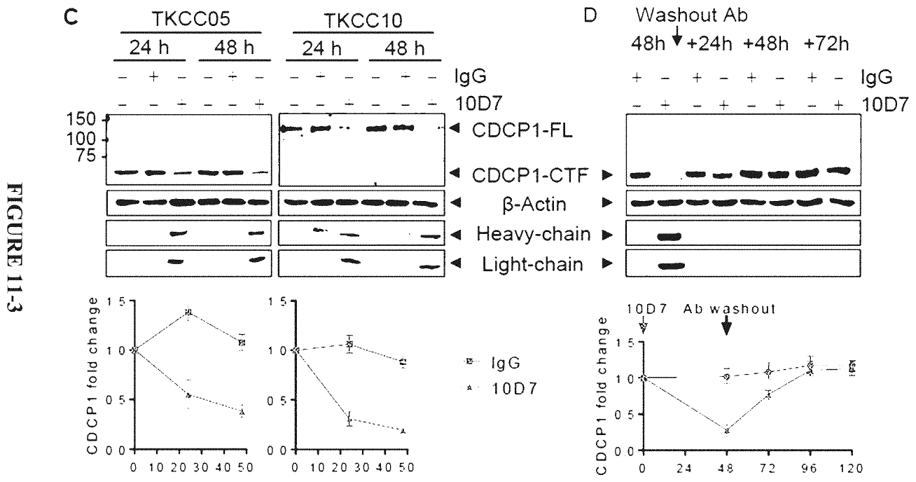

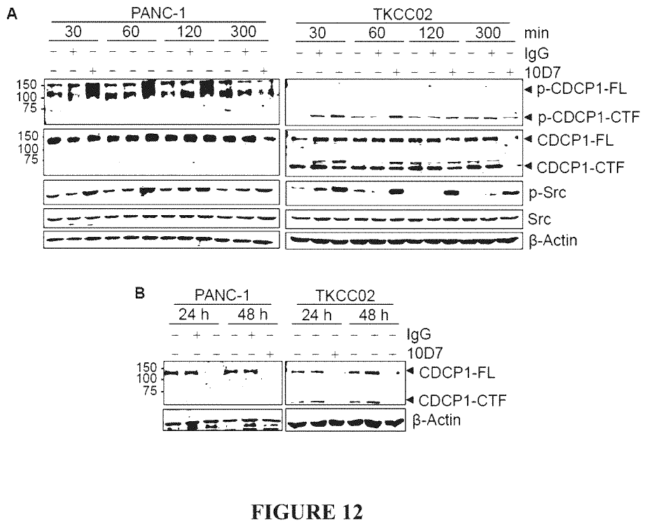

To assess the impact of antibody 10D7 on CDCP1 expressed by PDAC, the inventors performed confocal microscopy and western blot analysis of patient-derived PDAC cells treated with this antibody for defined periods of time. Focusing initially on TKCC05 cells, confocal microscopy revealed that despite CDCP1 being predominantly converted to CDCP1-CTF (which lacks the 10D7 binding site present within CDCP1-ATF), fluorescently labelled 10D7 (10D7-Qdot) was apparent on the plasma membrane within 5 minutes of the commencement of treatment, with strong signal apparent within 30 min, and after 120 min the antibody was largely internalized, observable as tight intracellular puncta (FIG. 11A). Western blot analysis confirmed that 10D7 induces rapid transient tyrosine phosphorylation of CDCP1-ATF in TKCC05 cells and CDCP1-FL in TKCC10 cells, while Src was also transiently tyrosine phosphorylated in these cells in response to 10D7 (FIG. 11B). Anti-CDCP1 western blot analyses indicated that by the 300 min time point that 10D7 treatments had started to reduce levels of CDCP1-CTF and CDCP1-FL (FIG. 11B). Consistent data were obtained from PANC-1 and TKCC02 cells, although analysis of the latter interestingly revealed that 10D7 binding to CDCP1-ATF induces most robust phosphorylation of CDCP1-CTF not CDCP1-FL (FIG. 12A). Western blot analysis also revealed that sustained treatment of PANC-1, TKCC02, TKCC05 and TKCC10 cells over 24 and 48 h with antibody 10D7 results in marked reduction in levels of both CDCP1-CTF and CDCP1-FL (FIG. 11C and FIG. 12B). Removal of 10D7 resulted in gradual re-expression of CDCP1 with a return to basal levels within 24-48 h in TKCC05 cells (FIG. 11D) indicating that the impact of 10D7 on CDCP1 protein levels in PDAC cells is reversible. These results collectively indicate that 10D7 is able to bind to intact CDCP1 and proteolytic fragments CDCP1-ATF/CDCP1-CTF and to induce downstream signalling, internalization of the receptor/antibody complex, and degradation of CDCP1. The data are consistent with CDCP1-ATF remaining tethered to the plasma membrane likely via CDCP1-CTF and indicate that intact and cleaved CDCP1 can be functionally targeted with antibody 10D7 in PDAC cells. Thus, antibody 10D7 is effective at targeting uncleaved as well as cleaved CDCP1 that are present on the surface of cancer cells.

### Example 11 Antibody Targeting of CDCP1 Reduces Cell Migration and Non-Adherent Growth, and Improves Chemo-Responsiveness of PDAC Cells In Vitro

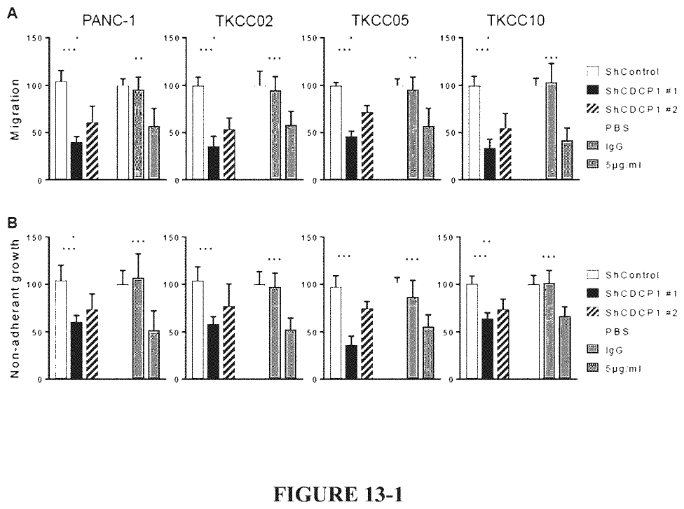

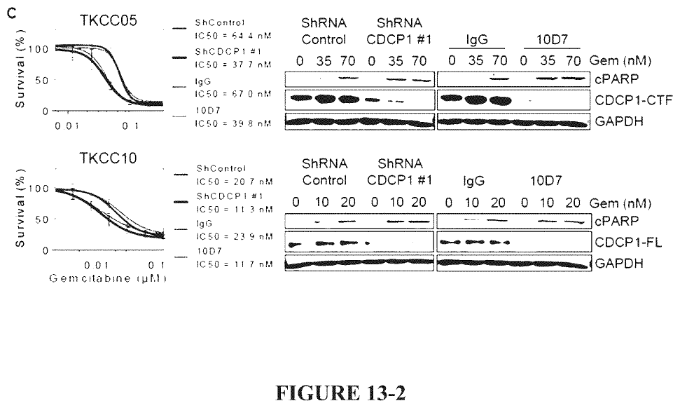

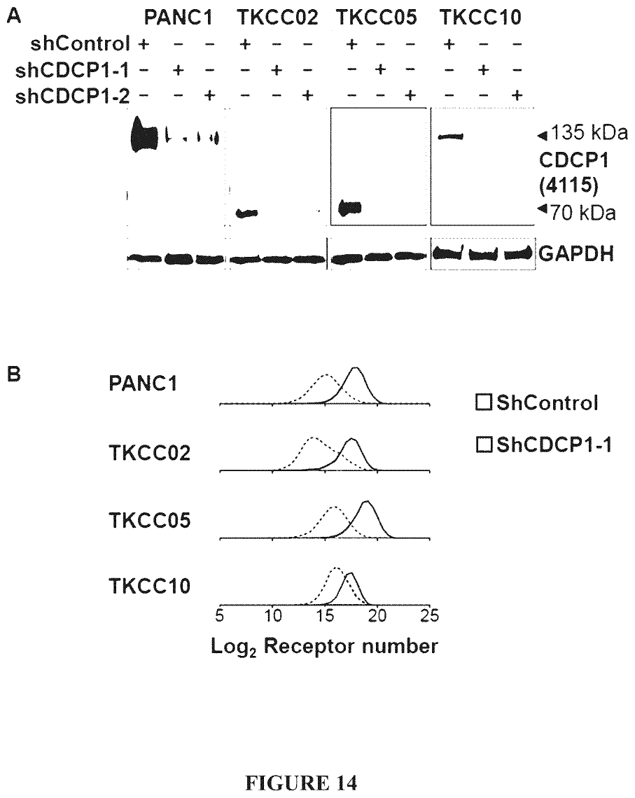

To directly evaluate whether functional targeting of CDCP1 can inhibit cellular processes that contribute to progression of PDAC, the inventors employed antibody 10D7 in in vitro models assessing effects on cell migration, non-adherent growth and chemo-resistance. In the PDAC cell line PANC-1 and the patient-derived lines TKCC02, TKCC05 and TKCC10, treatment for 48 h with 10D7 reduced migration by about 50-65% which was consistent with reductions achieved by stable silencing of CDCP1 with two lentiviral shRNA constructs (FIG. 13A). Western blot and flow cytometry analysis demonstrated the effectiveness of the silencing constructs at reducing total and cell surface expression of CDCP1, respectively (FIGS. 14A and 14B). Non-adherent cell growth in serum free, growth factor defined media, as a read-out for the presence of PDAC stem cell populations (Gaviraghi M et al (2011) Biosci Rep 31(1):45-55), saw a similar reduction in the number of actively dividing cell spheroids after 10 days in response to antibody 10D7 and this was also closely mimicked by stable silencing of CDCP1 (FIGS. 13B and 14B). Functional blockade of CDCP1 with 10D7 for 72 h also increased the in vitro efficacy of a chemotherapy commonly used in the treatment of PDAC, gemcitabine, halving the GI50 value achieved for TKCC05 and TKCC10 cells, with similar results obtained from silencing of CDCP1 (FIG. 13C, left). Interestingly, the improved efficacy of gemcitabine caused by 10D7 was accompanied by a marked increase in cell death as evidenced by increasing levels of cleaved PARP even though CDCP1 had been largely degraded in response to continuous treatment with the antibody for 72 h (FIG. 13C, right). These data indicate that independent of the state of CDCP1 cleavage, 10D7 is effective at targeting PDAC cells in vitro.

### Example 12 10D7 Antibody Specifically Detects PDAC Cells In Vivo

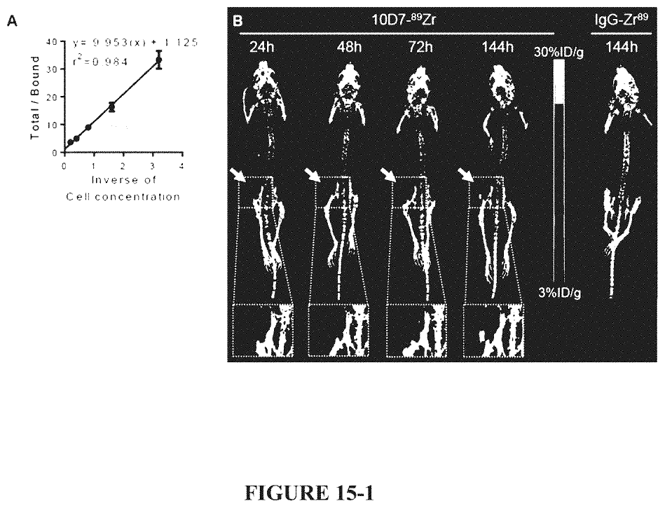

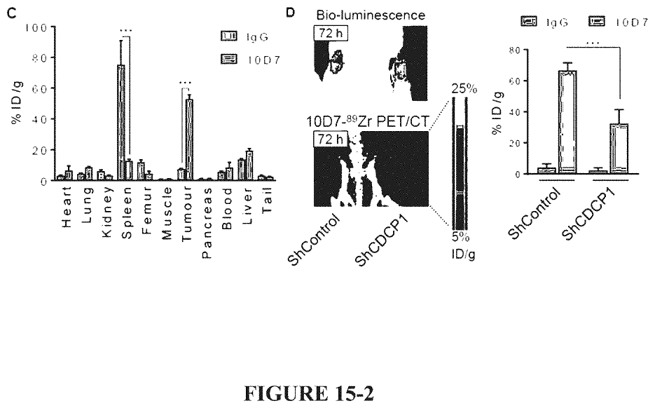

To evaluate whether the ability of antibody 10D7 to disrupt cellular processes that promote PDAC in vitro can be harnessed to target this cancer in vivo, the inventors first evaluated the capacity of this antibody to detect PDAC xenografts in mice. This was performed by immuno-PET/CT imaging of mice bearing subcutaneous xenografts of TKCC05 cells. Antibody 10D7 was efficiently conjugated with the positron emitting radionuclide ⁸⁹Zr as indicated by Lindmo assay analysis demonstrating an intact 10D7-⁸⁹Zr IRF of 88.9% (FIG. 15A). Imaging was performed on relatively small (˜100 mm³) tumours two weeks after subcutaneous injection of TKCC05 cells into host mice. Two randomised groups of mice (n=3/group) were administered intravenous 10D7-⁸⁹Zr or IgG-⁸⁹Zr (average dose 1.4 MBq/mouse) and PET/CT imaging was performed at 24, 48, 72 and 144 h time points. A strong time-dependent accumulation in tumours of 10D7-⁸⁹Zr but not IgG-⁸⁹Zr was observed with signal clearly visible at 24 h in tumours of all mice administered 10D7-⁸⁹Zr (FIG. 15B). Experimental endpoint radioactivity biodistribution analysis confirmed that 10D7-⁸⁹Zr, in contrast with IgG-⁸⁹Zr, predominantly accumulated in tumours (53.1% ID/g versus 7.6% ID/g; FIG. 15C). Non-specific accumulation of IgG-89Zr was particularly strong in spleen (76% ID/g for IgG-⁸⁹Zr versus 13% ID/g for 10D7-⁸⁹Zr), and both 10D7-⁸⁹Zr and IgG-⁸⁹Zr accumulated non-specifically at lower levels in liver and femur, sites which are commonly observed in mouse models (FIG. 15C). To confirm that 10D7-⁸⁹Zr accumulation in PDAC tumours is dependent on CDCP1 expression, the inventors compared the signal obtained from subcutaneous tumours of TKCC05 cells stably transduced with CDCP1 silencing or control lentiviral constructs. CDCP1 silenced and control tumours in the flanks of mice grew at the same rates (FIG. 15D, left upper panel) and displayed 10D7-⁸⁹Zr signal in tumours in proportion to the level of expression of CDCP1 (37.7% ID/g vs 66.7% ID/g, FIG. 15D left lower and right panels). As expected, minimal signal was observed in tumours of mice administered IgG-⁸⁹Zr (FIG. 15D, right panel). These data demonstrate the ability of antibody 10D7 to selectively target CDCP1 expressing PDAC tumours in vivo.

### Example 13 Antibody Targeting of CDCP1 Reduces Tumour Burden and Improves Gemcitabine Efficacy In Vivo

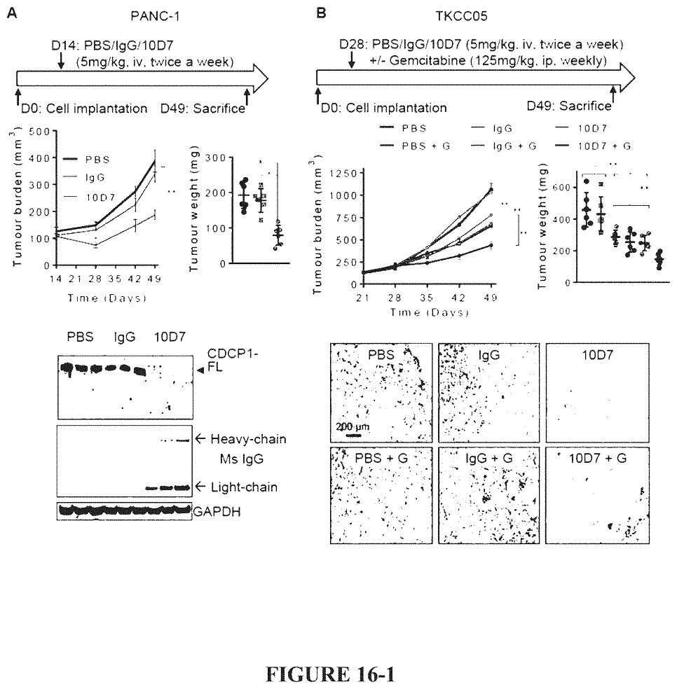

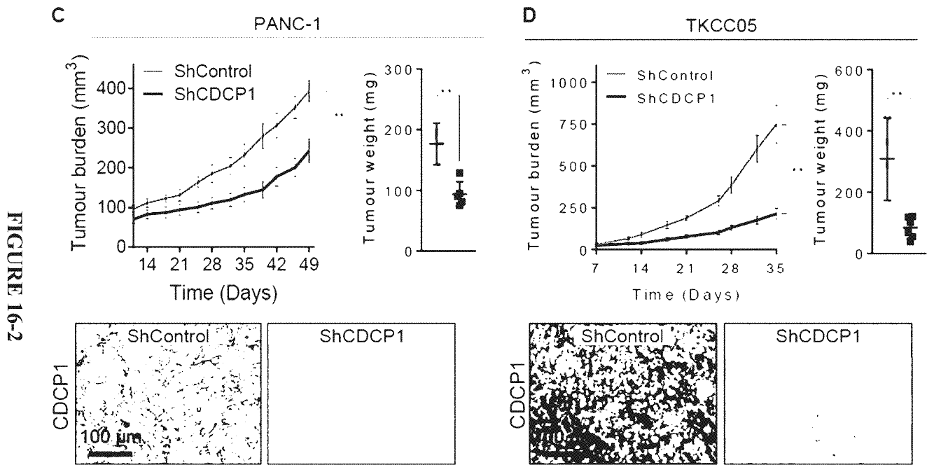

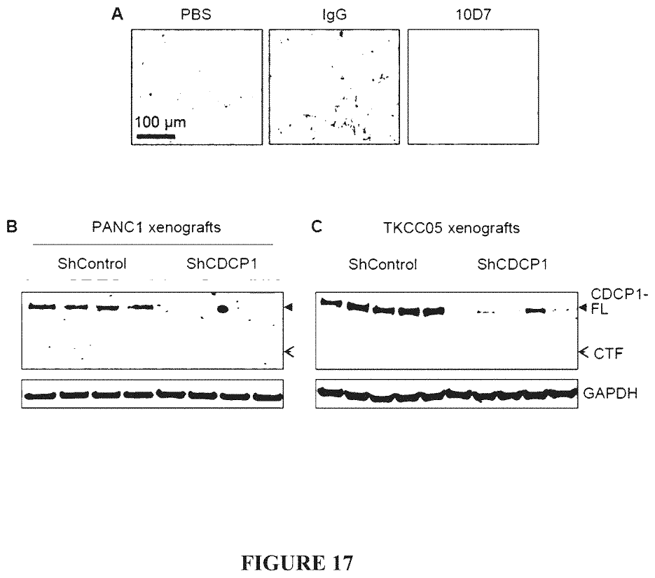

The inventors examined whether the ability of antibody 10D7 to disrupt PDAC cells in vitro and detect PDAC tumours in vivo, can be harnessed to inhibit growth of established subcutaneous xenografts in mice of luciferase expressing PANC-1 and TKCC05 cells. Twice weekly treatments with 10D7 (5 mg/kg) for 5 weeks significantly slowed growth of PANC-1 tumours and reduced end-point tumour weight by about 60% compared with treatments with isotype matched IgG and PBS (FIG. 16A, upper panels). Consistent with results from in vitro assays (FIG. 12B), 10D7 treatments markedly reduced CDCP1 levels in PANC-1 xenografts as assessed by western blot (FIG. 16A, lower panel) and immunohistochemical (FIG. 17A) analysis. For TKCC05 cell xenografts the inventors examined whether antibody 10D7 improves the efficacy in vivo of gemcitabine. Six treatments with 10D7 (5 mg/kg) every four days over 20 days in combination with two weekly treatments with the chemotherapy (100 mg/kg) significantly slowed the growth of subcutaneous xenografts of TKCC05 cells (FIG. 16B, upper left panel). At end-point the combination resulted in about 30% reduction in tumour weight compared with treatments with the single agents or PBS (FIG. 816B, upper right panel). As seen in PANC-1 xenografts, 10D7 treatments reduced the level of CDCP1 expression by TKCC05 cell xenografts (FIG. 16B, lower panel). These data are consistent with findings from in vitro assays showing that antibody 10D7 has anti-PDAC effects and that it results in degradation of CDCP1 (FIG. 16). Also, consistent with effects on tumour burden caused by 10D7, stable silencing of CDCP1 markedly reduced tumour burden of subcutaneous xenografts of PANC-1 and TKCC05 cells compared with xenografts of these cells stably transduced with scramble control vectors (FIGS. 16C and D and FIGS. 17B and C.

### Example 14 Antibody 10D7 is Effective for Specific Cytotoxin Delivery to PDAC Cells In Vitro and In Vivo

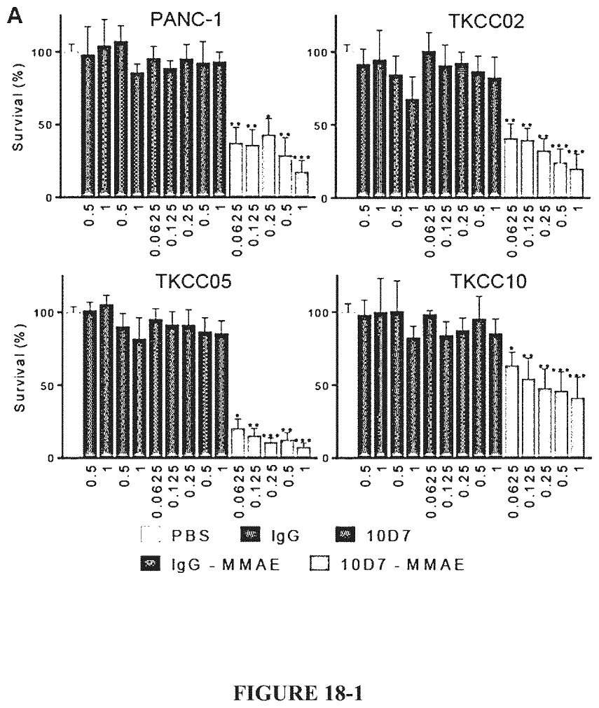

To assess the ability of 10D7 to deliver cytotoxic payloads to PDAC, the inventors labelled it and isotype matched control IgG with the highly potent toxin MMAE via a link incorporating a lysosomal protease cleavage site that promotes intracellular release of the toxin and cell death (Kamath A V, et al., (2015) Pharm Res. 32(11):3470-9). The generated antibody-drug conjugate (ADC), 10D7-MMAE, has an average drug-antibody ratio (DAR) of 4.5 to 4.7, and retains the functional ability to induce phosphorylation of CDCP1 and Src (see Example 7). 10D7-MMAE significantly reduced survival in vitro in a dose-dependent manner of PANC-1, TKCC02, TKCC05 and TKCC10 cells compared with IgG, 10D7 and IgG-MMAE controls (FIG. 18A). Of note, comparing the naked 10D7 antibody and 10D7-MMAE, at a concentration of 1 μg/ml, survival of PANC-1, TKCC02, TKCC5 and TKCC10 cells reduced, respectively, from about 85% to about 20%, about 75% to about 25%, about 85% to about 10% and about 80% to about 50% (FIG. 18A). The relative resistance of TKCC10 cells to 10D7-MMAE may relate to its lower level of CDCP1 expression or an as yet unidentified cellular mechanism by which this cell type processes 10D7-MMAE with lower efficiency. The selectivity of 10D7-MMAE for CDCP1 expressing cells was confirmed by treatment of co-cultures of CDCP1 expressing TKCC05 cells and non-expressing normal human pancreatic stellate cells (FIG. 18C left; red and green cells, respectively). Whereas the stellate cells were unresponsive to 10D7-MMAE, TKCC05 cells were very sensitive to this agent (FIG. 18B, right).

The selectivity of 10D7-MMAE for CDCP1 expressing cells was confirmed by treatment of co-cultures of CDCP1 expressing TKCC05 cells and non-expressing normal human pancreatic stellate cells (FIG. 18B left; red and green cells, respectively). Whereas the stellate cells were unresponsive to 10D7-MMAE, TKCC05 cells were very sensitive to this agent (FIG. 18B right).

Finally, the inventors evaluated whether the PDAC targeting ability of antibody 10D7 can be harnessed to effectively deliver the cytotoxin MMAE to PDAC in vivo. Mice with established subcutaneous TKCC05 cells xenografts were treated on day 27 and 41 with 10D7-MMAE, the naked 10D7 antibody or IgG labelled labelled with MMAE (IgG-MMAE), or on day 27, 34, 41 and 48 with gemcitabine. Of significance, 10D7-MMAE markedly inhibited tumour growth (FIG. 18C) and significantly extended survival of xenografted mice (FIG. 18E) in comparison with the other treatments.

### Example 15 Chimeric 10D7 Antibody Detects Human Tumour Xenografts in Mice

The mouse 10D7 antibody described in examples 1-14 was modified to replace the constant region sequences of the heavy and light chains with corresponding human sequences. As demonstrated in FIG. 19, the human/mouse chimeric 10D7 antibody bound CDCP1 expressing TK0005 pancreatic cancer cells as demonstrated by flow cytometry.

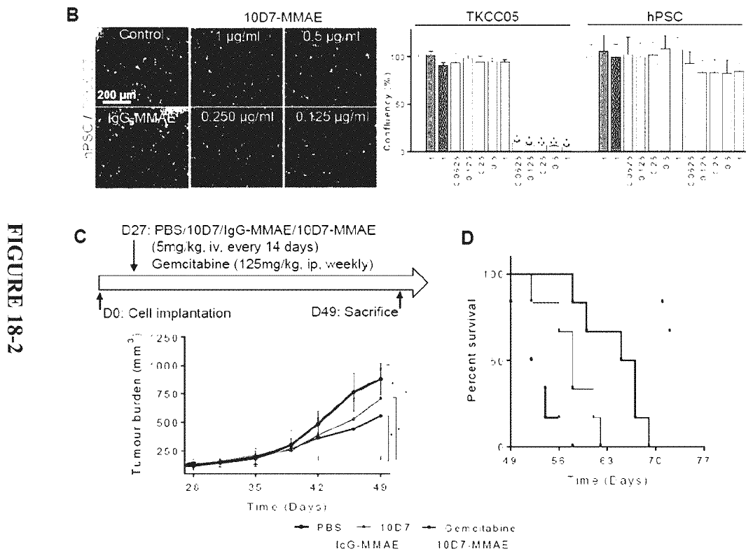

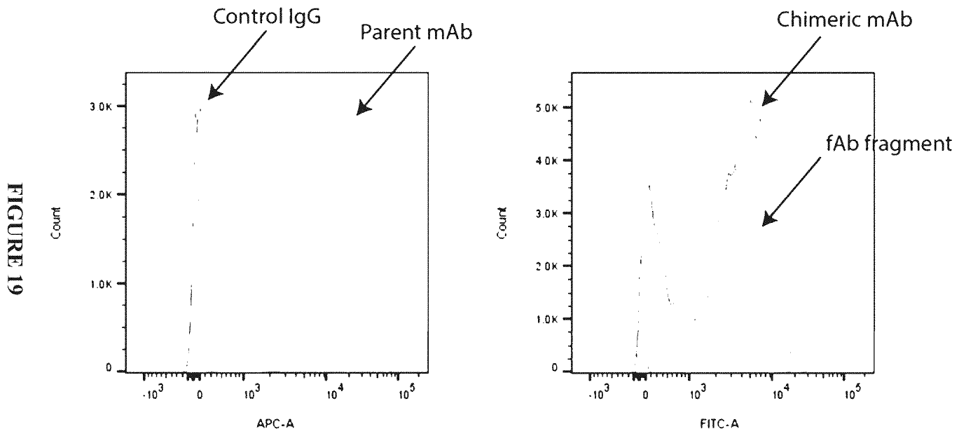

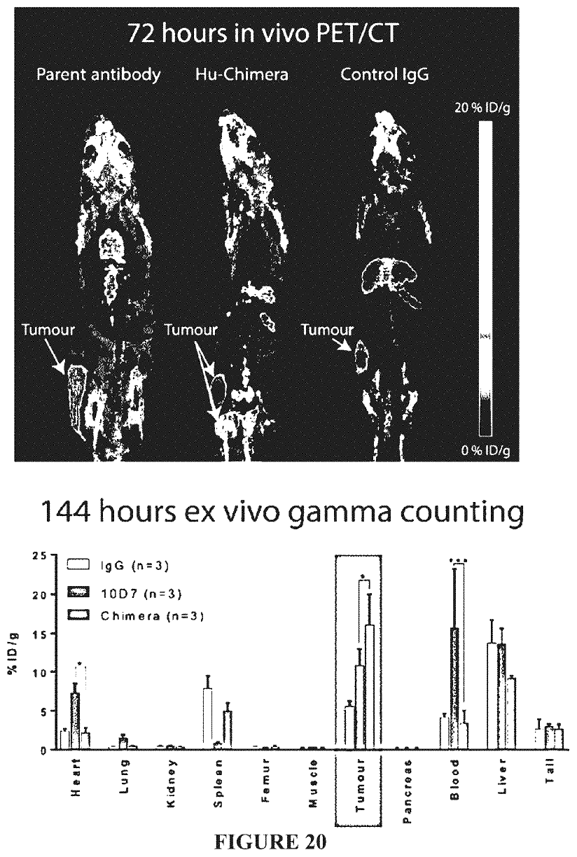

PET/CT imaging was used to assess the efficacy with which the chimeric 10D7 antibody detected subcutaneous human TKCC02 xenograft tumours in mice. The chimeric antibody was labelled with the positron-emitting radionuclide ⁸⁹Zr as previously described (Zeglis B M et al., (2015) Jove-J Vis Exp (96): 52521). The labelled antibody was injected intravenously (3-5 MBq) into female NOD. Cg-Prkdc^{scid}Il2rg^{tm1W}/SzJ mice bearing subcutaneous xenografts of human TKCC02 pancreatic tumours. Imaging was performed on a Siemens Inveon preclinical PET/CT at 1, 24, 48, 72 and 144 hours post-injection. The data presented in FIG. 20 (top) show that the chimeric 10D7 antibody was able to detect CDCP1 expressing tumours in vivo. Furthermore, the chimeric 10D7 antibody was able to accumulate in the tumour to a significantly greater degree than the original parent mouse 10D7 antibody (FIG. 20 bottom).

### Example 16 In Vitro Activity of 10D7 and Chimeric 10D7 Conjugates

Mouse 10D7 antibody and chimeric (chim) 10D7 antibody (comprising human heavy and light chain constant region sequences) were conjugated with the cytotoxin MMAE. TKCC05 pancreatic cancer cells (expressing CDCP1 mainly under its cleaved form) were plated at 5,000 cells per well (in a 96 well plate) and treated with various concentrations (31.25 to 2,000 ng/ml) of antibody-drug conjugate (IgG-MMAE, isotype control antibody; 10D7-MMAE; Chim-10D7-MMAE). After 18h treatment, medium was replaced with growth medium not containing any treatment. Cell survival was measured in real time by imaging using Incucyte S3 instrument (Essence Bioscience) and is expressed as relative confluence in comparison to confluence of control cells treated with vehicle (PBS).

FIG. 21A shows the effect of 10D7-MMAE and Chim 10D7-MMAE (hu/mu-10D7-MMAE) on confluence of TKCC05 cells demonstrating the effectiveness of these constructs to induce cell killing as shown by less than 50% confluence of the cells at 96 hours. FIG. 21B shows confluence of TKCC05 cells expressed as confluence (%) relative to vehicle treated control cells.

### Example 17 Binding Capacity of 10D7 and Chimeric 10D7 to Cancer Cells Expressing Cleaved and Uncleaved CDCP1

Antibody 10D7, Chimeric hu/mu-10D7 and isotype control IgG antibodies were conjugated with fluorophore Atto-550 as per manufacturer instruction (Sigma, Atto 550 Protein labelling kit, 51146-1KT). Cancer cells (TKCC05, pancreatic cancer cells expressing CDCP1 mainly under its cleaved form; HEY, ovarian cancer cells expressing CDCP1 mainly under its full length form) were lifted using non-enzymatic solution (versene, to avoid receptor proteolysis) and blocked for 30 min using PBS+0.5% BSA (w/v).

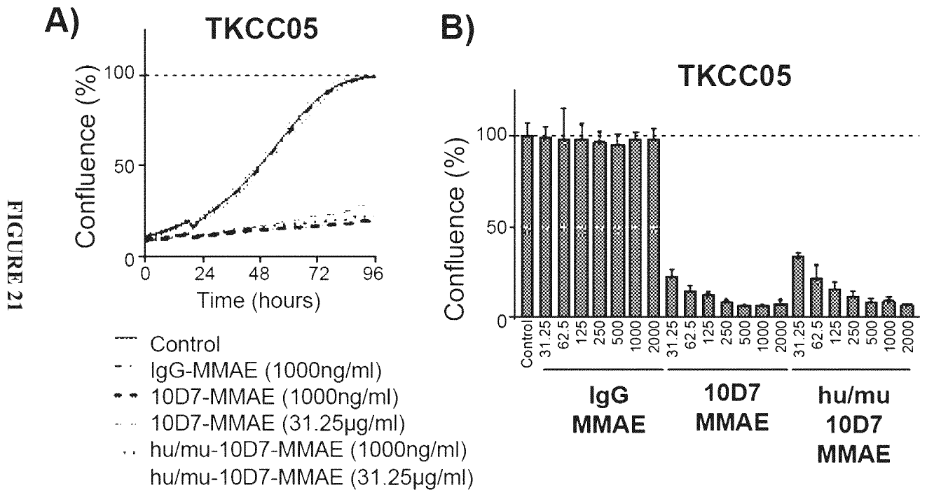

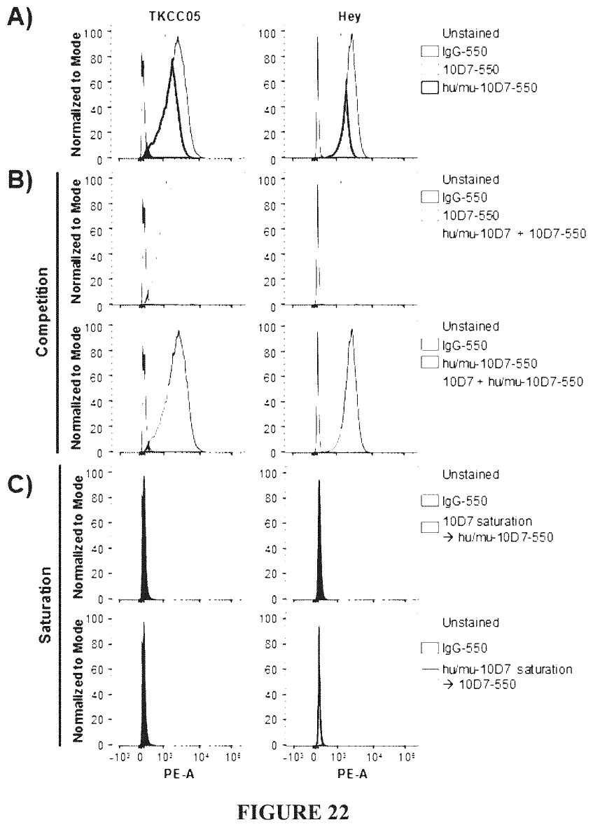

FIG. 22A shows the results of flow cytometry analysis wherein 100,000 cells were incubated 1 h at 4° C. with each of Atto-550 labelled antibody diluted in blocking buffer (final volume of 200 μl, final antibody concentration 10 ng/ml). Cells were washed 3 times using cold PBS before to be analysed by flow cytometry in parallel to unstained cells (incubated in blocking buffer in absence of antibody).

FIG. 22B shows flow cytometry competition experiments (using the same protocol as in FIG. 22A) but cells were incubated with mixture of labelled and unlabelled antibody at equal concentration (10D7-550+unlabelled hu/mu-10D7 or hu/mu-10D7-550+unlabelled 10D7).

FIG. 22C shows a saturation experiment, wherein after blocking, cells were incubated for an hour at 4° C. with an excess amount of unlabelled 10D7 or hu/mu-10D7 (200 ng/ml), then, after 3 washes with cold PBS, staining was performed as in FIG. 22A.

The results show that antibody 10D7 and human/mouse chimeric 10D7 antibody bind to CDCP1 expressing TKCC05 pancreatic cancer cells and HEY ovarian cancer cells, these antibodies compete for binding to CDCP1 expressing cells, and that saturating levels of each antibody blocks the other antibody binding to CDCP1 expressing cells, indicating that binding of the Complementarity-Determining Regions of 10D7 are retained in the human/mouse chimeric 10D7 antibody.

### Example 18 CDCP1 Degradation Assay in Ovarian Cancer Cells

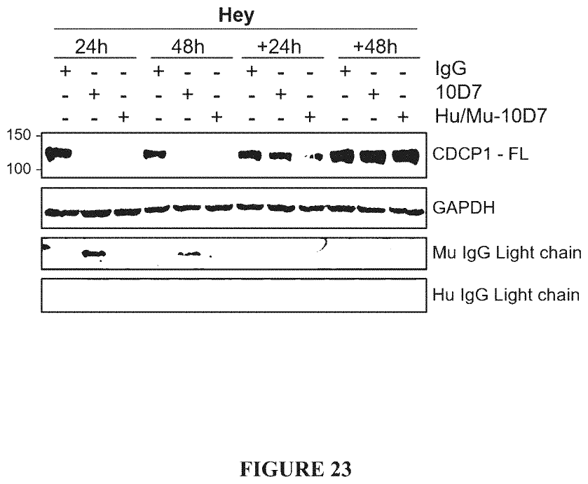

Ovarian cancer cells (HEY) were seeded in 6 well plate in growth medium (200,000 per well). Once confluence was reached about 70%, cells were treated with 5 μg/ml of isotype IgG control, 10D7 or chimeric hu/mu-10D7 antibody in a final volume of 2 ml. Cells were lysed using RIPA buffer after 24h and 48h treatment. In addition, to investigate the re-expression of CDCP1 after 48h antibody treatment, the medium was replaced with fresh growth medium not containing any treatment for 24 and 48h (+24h and +48h respectively) and cell were lysed in RIPA buffer. Protein lysates were quantified by BCA and Western blot were performed to analyse CDCP1 expression (anti-CDCP1 4115 antibody, Cell signalling) as shown in FIG. 23, GAPDH (loading control, Cell signalling) and level of internalized/bound antibody (using anti-mouse or anti-human IgG secondary antibody, Cell signalling).

### Example 19 Imaging of Pancreatic Tumours In Vivo

Four weeks after intra-pancreas injections (100,000 cells, TKCC05-luciferase cells expressing mainly cleaved form of CDCP1), mice received equivalent doses of either 10D7-89Zr, Chim hu/mu-10D7-89Zr or control IgG1K-89Zr via the lateral tail vein (˜1.5 MBq).

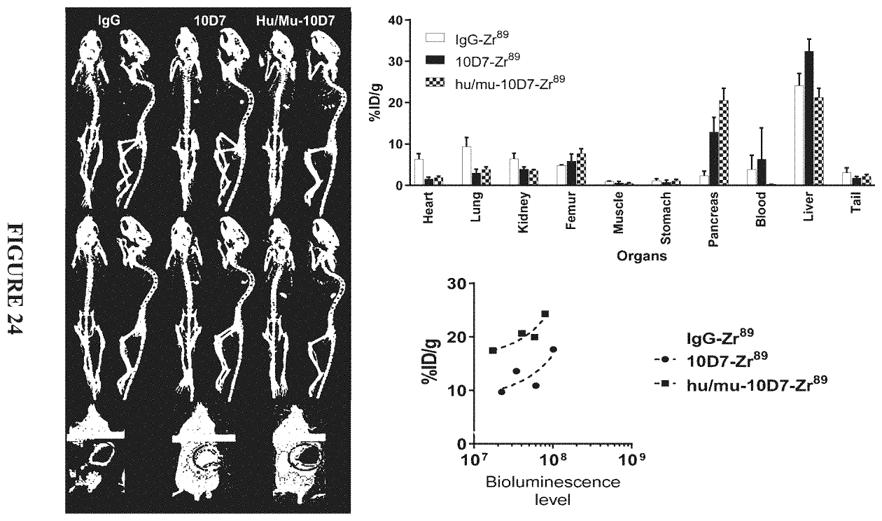

FIG. 24A shows PET-CT imaging performed on isoflurane anaesthetised mice after 144 h using an Inveon PET/CT unit (Siemens, Munich, Germany). PET acquisition (30 minutes; static emission) was performed, and images were reconstructed using an ordered-subset expectation maximization (OSEM2D) algorithm, with CT attenuation correction. The CT scan parameters were 80 kV, 500 μA, 230 ms exposure time, 360o rotation with 180 rotation steps, binning factor of 4, low magnification position, producing an effective pixel size of 106 μm, with CT images reconstructed using the Feldkamp algorithm. All PET and CT images were reconstructed using Inveon Acquisition Workplace software (Siemens). PET activity per voxel was converted to bq/cc using a conversion factor obtained by scanning a cylindrical phantom filled with a known activity of 89Zr to account for PET scanner efficiency. Activity concentrations within tissue ROIs were expressed as percentage of the decay-corrected injected activity per cubic cm of tissue (% ID/cc; SUV) using Inveon Research Workplace software (Siemens).

Ex vivo bio-distribution was assessed after the final imaging time point (FIG. 24B). Harvested tumor and organs, cleaned of blood, were weighed and radioactivity quantified using a Wizard 2480 gamma counter (Perkin Elmer) and presented as % ID/g of tumor or tissue (after decay and detector efficiency corrections).

FIG. 24C shows correlation analysis between Zr89 accumulation in pancreas 144h after administration of Zr89 labelled antibodies (% ID/g) and bioluminescence signal (arbitrary unit) from the pancreas area.

The results show that antibody 10D7 and the human/mouse chimeric 10D7 antibody, labelled with Zircodium-89, preferentially accumulate in intra-pancreatic pancreas cancer tumours, and that non-specific signal of human/mouse-10D7-⁸⁹Zr in liver is more rapidly cleared than 10D7-⁸⁹Zr.

### Example 20 Impact of MMAE-Labelled 10D7 and Chimeric 10D7 Antibody on Pancreatic Cancer Xenografts

Pancreatic cancer cells (TKCC2.1 cells, 1×10⁶ cells) were grafted subcutaneously in NSG mice. After 4 weeks, mice were randomized into 5 groups (10 mice per group) and treated with: PBS (iv, 125 μl fortnightly), IgG-MMAE (iv, 125 μg diluted in 125 μl PBS), 10D7-MMAE (iv, 125 μg diluted in 125 μl PBS), Chim hu/mu-10D7-MMAE (iv, 125 μg diluted in 125 μl PBS) or Gemcitabine (ip, 100 mg/kg in PBS weekly).

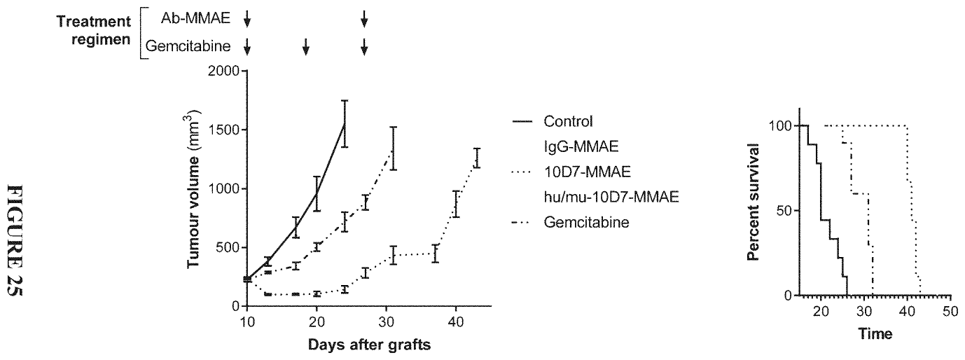

FIG. 25A shows tumour burden wherein tumour volume was calculated using calliper measurement (as previously described) performed twice a week.

FIG. 25B shows survival. Mice were euthanized when one ethical end point was reached (either tumour size or excessive signs of distress).

These results demonstrate that both 10D7 and chimeric hu/mu 10D7 MMAE conjugated antibodies were superior to gemcitabine (standard of care) in reducing pancreatic tumour volume. Moreover, survival of mice was prolonged in mice that received 10D7 or chimeric 10D7 antibodies. Tumour re-growth after day 35 suggests that more than two treatments with 10D7-MMAE and human/mouse-10D7-MMAE are likely required to eliminate tumour burden.

### Example 21 Measurement of Internalisation of 10D7 and Chimeric 10D7 Antibody in Pancreatic Cancer Cells

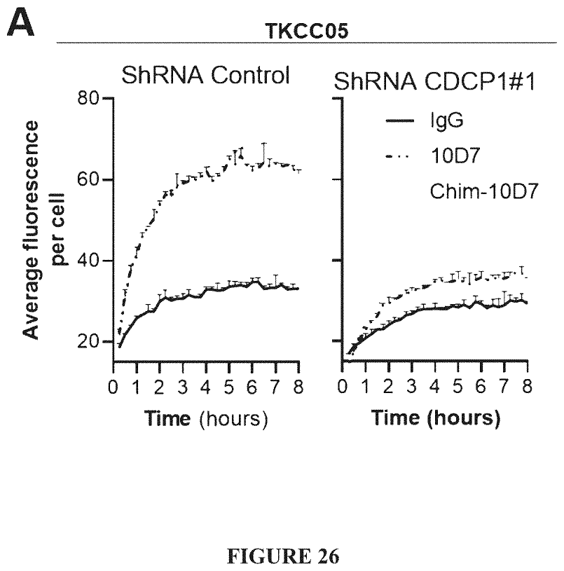

TKC005 pancreatic cancer cells (5,000 cells expressing control ShRNA-CCTAAGGTTAAGTCGCCCTCGCTCGAGCGAGGGCGACTTAACCTTAGG) or ShRNA-targeting CDCP1-CCGGGCTCATAAGAGCATCGGTTTACTCGAGTAAACCGATGCTCTTATGAGCTTTTTTG) were plated into 96 well plate in growth medium. When confluence reached ˜50%, cells were treated with 2 μg/ml of IgG isotype control, 10D7 or Chim hu/mu-10D7 pre-labelled with pH-sensitive fluorescent dye (FabFluor-pH Red Antibody Labelling Reagent, Essen-Biosciences). Fluorescence per cells were measured in real time using Incucyte S3 over 8h with scanning every 15 min. Results are expressed as average fluorescence per cell (+/−SD) as determined by Incucyte instrument and shown in FIG. 26.

### Remarks

These data demonstrate that antibody-mediated targeting of the receptor CDCP1 is effective to deliver cytotoxins to kill ovarian cancer cells in vitro and PDAC cells in vitro and in vivo, and to deliver positron-emitting radionuclides for PET imaging of ovarian cancer and PDAC xenografts in mice. Of note, antibody 10D7 is as effective at delivering imaging and cytotoxic agents to cancer cells that predominantly express intact CDCP1 as it was to cells that predominantly express cleaved CDCP1. Thus, antibody 10D7 and variants derived from it, have potential to be effective at detecting and treating all tumours that express intact CDCP1 as well as those that express cleaved CDCP1. Another key finding is that CDCP1 is functionally important in several cellular processes that promote ovarian and pancreatic cancer including cell migration, non-adherent cell growth, resistance to gemcitabine chemotherapy and primary tumour growth, each of which contributes to progression of these cancers in vivo.

In particular, the in vitro and mouse assays indicate that the function blocking anti-CDCP1 antibody 10D7 disrupts processes that promote progression of ovarian cancer and PDAC. Of note, a “weaponized” form of antibody 10D7, conjugated with the highly toxic agent MMAE, displays significant ability to induce death of ovarian cells in vitro and PDAC cells in vitro and in vivo and a humanized form of cytotoxin-conjugated 10D7, likely due to the rapid internalisation of the CDCP1-10D7 complex and, as such, will likely have clinical utility for treatment of cancers that express uncleaved CDCP1 or cleaved CDCP1. The finding that CDCP1 expression is elevated in the vast majority of ovarian and PDAC patient tumours, but is not expressed by normal ovary or pancreas, supports CDCP1 as a target for delivery of agents that could assist in the prognostication and treatment of ovarian cancer and PDAC.

1. An anti-CDCP1 antibody or antigen-binding fragment thereof which specifically binds to CDCP1, comprising a heavy chain variable region (VH) having complementarity determining region (CDR) sequences comprising the sequences set forth in GYSFSDFN (SEQ ID NO:4), INPNYDST (SEQ ID NO:5), ARLGYGYAMDY (SEQ ID NO:6) respectively and/or a light chain variable region (VL) having complementarity determining region (CDR) sequences comprising the sequences set forth in ENVGAY (SEQ ID NO:7), AAS (SEQ ID NO:8) and GQSYTYPYT (SEQ ID NO:9).

2. The antibody or antigen-binding fragment thereof according to claim 1, which binds to the cleaved CDCP1 receptor or the uncleaved CDCP1 receptor.

3. The antibody or antigen-binding fragment thereof according to claim 1 or 2, wherein upon binding the anti-CDCP1 antibody or antigen-binding fragment thereof and receptor are internalised.

4. The antibody or antigen-binding fragment thereof according to any one of claims 1 to 3, wherein the antibody or antigen-binding fragment thereof substantially improves the efficacy of chemotherapy compared to administration of the chemotherapy or antibody or antigen-binding fragment thereof alone.

5. The antibody or antigen-binding fragment thereof according to any one of claims 1 to 4, comprising a VH comprising a sequence which is at least 86% identical to the sequence set forth in SEQ ID NO:2 and/or a VL comprising a sequence which is at least 92% identical to the sequence set forth in SEQ ID NO:3.

6. The antibody or antigen-binding fragment thereof according to claim 5 further comprising a heavy chain constant region sequence comprising the sequence set forth in SEQ ID NO:10 and/or a light chain constant region sequence comprising the sequence set forth in SEQ ID NO:11.

7. A nucleic acid sequence encoding a heavy chain variable (VH) region sequence and/or a light chain variable (VL) region sequence of the antibody or antigen-binding fragment thereof according to any one of claims 1 to 6.

8. An expression construct comprising the nucleic acid sequence according to claim 7 operably linked to a promoter.

9. An isolated cell comprising the expression construct according to claim 8.

10. The anti-CDCP1 antibody or antigen-binding fragment thereof according to any one of claims 1 to 6 coupled to a detectable label.

11. The anti-CDCP1 antibody or antigen-binding fragment thereof according to any one of claims 1 to 6 coupled to a moiety selected from the group consisting of an anti-apoptotic agent, a mitotic inhibitor, an anti-tumour antibiotic, an immunomodulating agent, a nucleic acid for gene therapy, an anti-angiogenic agent, an anti-metabolite, a toxin, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, and a tyrosine kinase inhibitor.

12. An antibody drug conjugate (ADC) comprising an antibody conjugated to at least one drug, wherein the antibody or antigen-binding fragment thereof comprises a heavy chain variable (VH) sequence comprising the sequence set forth in SEQ ID NO:2 and/or a light chain variable (VL) sequence comprising the sequence set forth in SEQ ID NO:3.

13. The ADC according to claim 12, wherein the drug is a cytotoxic or cytostatic agent.

14. The ADC according to claim 12 or 13 comprising the formula Ab-[L-D]n, wherein Ab comprises the antibody or antigen-binding fragment thereof according to any one of claims 1 to 6, wherein L comprises an optional linker; D is a therapeutic agent; and n is an integer from about 1 to about 20.

15. A pharmaceutical composition comprising the anti-CDCP1 antibody or antigen-binding fragment thereof according to any one of claims 1 to 6, the labelled anti-CDCP1 antibody or antigen-binding fragment thereof according to claim 10, or the ADC according to claim 12 or 13 together with a pharmaceutically acceptable carrier.

16. A pharmaceutical composition comprising an ADC mixture the ADC mixture comprising a plurality of the ADC according to any one of claims 12 or 14 and a pharmaceutically acceptable carrier.

17. A method for detecting a CDCP1-expressing cancer cell in vitro or in vivo, the method comprising contacting the cancer cell in a subject or in a biological sample obtained from the subject with the antibody or antigen-binding fragment according to claim 10 and detecting binding of the anti-CDCP1 antibody or antigen-binding fragment thereof to the cancer cell or sample, thereby detecting CDCP1.

18. A method for treating a CDCP1 expressing cancer, comprising administering a therapeutically effective amount of the antibody or antigen binding fragment according to any one of claims 1 to 6, the labelled antibody according to claim 10, or 11, the ADC according to any one of claims 12 to 14, the composition according to claim 15 or 16, or the nucleic acid according to claim 7 to a subject in need thereof.

19. A method for inhibiting or decreasing solid tumour growth in a subject having a solid tumour, the method comprising administering a therapeutically effective amount of the antibody or antigen binding fragment thereof according to any one of claims 1 to 6, the labelled antibody according to claim 10, or 11, the ADC according to any one of claims 12 to 14, the composition according to claim 15 or 16, or the nucleic acid according to claim 7 to the subject having the solid tumour, such that the solid tumour growth is inhibited or decreased.

20. Use of an anti-CDCP1 antibody or antigen-binding fragment thereof according to any one of claims 1 to 6, the labelled antibody according to claim 10, or 11, the ADC according to any one of claims 12 to 14, the composition according to claim 15 or 16, or the nucleic acid according to claim 7 in the manufacture of a medicament for the treatment or prophylaxis of cancer.

21. A method for cancer prophylaxis comprising administering to a subject in need thereof the antibody or antigen-binding fragment thereof according to any one of claims 1 to 6, the labelled antibody according to claim 10, or 11, the ADC according to any one of claims 12 to 14, the composition according to claim 15 or 16, or the nucleic acid according to claim 7 in a prophylactically effective amount.

22. A cancer theranostic agent, comprising the anti-CDCP1 antibody or antigen-binding fragment thereof according to any one of claims 1 to 6 or 11 coupled to a radionuclide.

23. The agent according to claim 22, wherein the radionuclide is selected from one or more of the group consisting of ¹¹C, ¹⁸F, ⁶¹Cu, ⁶⁴Cu, ⁶⁸Ga, ⁸⁶Y, ⁸⁹Zr, ¹²⁴I, ¹¹¹In, ²¹³Bi, ¹⁶⁶Ho, ¹³¹I, ²⁰³Pb, ²¹²Pb, ¹⁷⁷Lu, ²²³Ra, ¹⁸⁶Re, ¹⁵³Sm, ⁸⁹Sr, ²²⁷Th, ⁹⁰Y, ²²⁵Ac, ²¹¹At, ⁶⁷Cu.

24. An immunotherapeutic agent comprising the anti-CDCP1 antibody or antigen binding fragment thereof according to any one of claims 1 to 6, wherein the immunotherapeutic agent comprises an immune cell modified to contain a chimeric antigen receptor (CAR).
